(19) United States
(12) Patent Application Publication
(10) Pub. No.: US 2025/0020629 A1
(43) Pub. Date: Jan. 16, 2025

(54) SEMICONDUCTOR DEVICES TO MEASURE ELECTRICAL SIGNALS OF MATERIAL DISPOSED IN FLUID

(71) Applicants: Analog Devices, Inc., Wilmington, MA (US); Analog Devices International Unlimited Company, Limerick (IE)

(72) Inventors: Phillip Michel Nadeau, Cambridge, MA (US); David Duncan Lloyd, Menlo Park, CA (US); Michael C.W. Coln, Lexington, MA (US); Roman Trogan, Needham, MA (US); Junfei Xia, Andover, MA (US); Marek Hempel, Boston, MA (US); Chen Yang, Lexington, MA (US); Ramji Sitaraman Lakshmanan, Limerick (IE)

(21) Appl. No.: 18/769,215

(22) Filed: Jul. 10, 2024

Related U.S. Application Data
(60) Provisional application No. 63/525,905, filed on Jul. 10, 2023.
(60) Provisional application No. 63/583,749, filed on Sep. 19, 2023.
(60) Provisional application No. 63/621,956, filed on Jan. 17, 2024.

(51) Int. Cl. G01N 33/483; B01L 3/00; H01L 21/311

(52) U.S. Cl. CPC G01N 33/4836; B01L 3/5088; B01L 2300/0645; B01L 2300/0819; B01L 2300/16; H01L 21/311

(57) ABSTRACT

Semiconductor devices can include features to apply electrical stimulation to liquids in which material is present and to measure the response to the electrical stimulation.

### PRIORITY CLAIM

This application claims priority to U.S. provisional patent application No. 63/525,905 filed on Jul. 10, 2023, and entitled System-On-Chip and Related Methods for High-Throughput Automated Patch Claim, U.S. provisional patent application No. 63/583,749 filed on Sep. 19, 2023, and entitled Methods of Manufacturing and Assembly for a High-Throughput Automated Patch Claim, and U.S. provisional patent application No. 63/621,956 filed Jan. 17, 2024, and entitled Compound Delivery Methods for an Automated Patch Claim System, each of which are incorporated by reference herein in their entireties.

### FIELD OF THE DISCLOSURE

This document pertains generally, but not by way of limitation, to apparatuses and methods related to measuring electrical signals of material disposed in fluid using semiconductor devices.

### BACKGROUND

Electrical stimulation can involve applying at least one of current or voltage to a substance. The response of the substance to the electrical stimulation can indicate characteristics of the substance. For example, an amount of conductivity of a substance can be determined in response to applying one or more electrical stimuli to the substance. In various examples, changes in electrical properties of a substance in response to one or more electrical stimuli can also indicate characteristics of the substance. To illustrate, some substances can undergo changes in composition and/or changes in form in response to one or more electrical stimuli. In one or more scenarios, the response of substances to electrical stimulation can be measured when the substances are disposed in fluids. In some examples, an automated patch clamp system can cause electrical stimuli to be applied to biological cells disposed in fluid. The electrical response of the biological cells to the electrical stimuli can indicate characteristics of the biological cells.

### SUMMARY OF THE DISCLOSURE

In one or more examples, a semiconductor device can include a substrate having one or more layers and one or more sites formed by at least a portion of the one or more layers. The one or more layers of the substrate can include a complementary metal oxide semiconductor (CMOS) layer. The CMOS layer can include circuitry to apply at least one voltage or current to a plurality of electrodes electrically connected to the circuitry. In addition, the circuitry can measure signals produced in response to the at least one of voltage or current being applied to the plurality of electrodes. Individual sites of the one or more sites can be configured to hold a discrete amount of liquid. Individual sites of the one or more sites can include one or more electrodes of the plurality of electrodes. The one or more electrodes of an individual site can contact the discrete amount of liquid disposed in the individual site. Individual sites of the one or more sites can also include one or more pores to provide fluid communication between the discrete amount of liquid and one or more additional liquids stored by the individual site.

In one or more examples, a process to manufacture a semiconductor device includes providing a substrate having one or more layers. The one or more layers of the substrate can include a CMOS layer. The CMOS layer can have a first surface and a second surface disposed at least substantially parallel with respect to the first surface. Circuitry can be formed at least one of on or within the CMOS layer. The circuitry can apply at least one of voltage or current to a plurality of electrodes included in the semiconductor device. The circuitry can also measure signals produced in response to the at least one of voltage or current being applied to the plurality of electrodes. In addition, the process can include forming one or more passivation layers on at least one of the first surface of the CMOS layer or the second surface of the CMOS layer. The process can also include forming a number of sites using the CMOS layer. Individual sites of the number of sites can be configured to hold a discrete amount of liquid and can include one or more electrodes of the plurality of electrodes. The plurality of sites can also be formed by producing a plurality of pores within the one or more passivation layers. Individual sites of the number of sites can include one or more pores of the plurality of pores.

In one or more examples, a process can include providing a semiconductor device that includes a substrate having a complementary metal oxide semiconductor (CMOS) layer that forms a number of sites. Individual sites of the number of sites can be configured to perform patch clamp operations. The process can also include providing individual amounts of a liquid to the individual sites. In addition, the process can include applying a pressure differential to cause one or more biological cells disposed in the individual amounts of liquid corresponding to the individual sites to move to a location of a pore included in the individual sites and to be in contact with an electrode of the individual sites. Further, the process can include contacting the individual amounts of liquid with individual amounts of one or more compounds and applying at least one of a voltage or a current to the individual amounts of liquid. One or more signals produced in response to applying the at least one of voltage or current to the individual amounts of liquid can be measured.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various implementations discussed in the present document.

FIG. 1 is a diagram depicting a framework for applying electrical stimulation to substances disposed in liquid using semiconductor devices, in accordance with one or more example implementations.

FIG. 2 is a diagram depicting a cross-section of at least a portion of a first example semiconductor device in which at least one of current or voltage can be applied to substances disposed in liquid, in accordance with one or more example implementations.

FIG. 3 is a diagram depicting a process of manufacturing a first example semiconductor device in which at least one of current or voltage can be applied to substances disposed in liquid, in accordance with one or more example implementations.

FIG. 4 is a diagram depicting a top view of a first example semiconductor device in which at least one of current or voltage can be applied to substances disposed in liquid, in accordance with one or more example implementations.

FIG. 5 is a diagram depicting a cross-section of at least a portion of a second example semiconductor device in which at least one of current or voltage can be applied to substances disposed in liquid, in accordance with one or more example implementations.

FIG. 6 is a diagram depicting a cross-section of at least a portion of a modified version of the second example semiconductor device in which at least one of current or voltage can be applied to substances disposed in liquid, in accordance with one or more example implementations.

FIG. 7 is a diagram depicting a process of manufacturing a second example semiconductor device in which at least one of current or voltage can be applied to substances disposed in liquid, in accordance with one or more example implementations.

FIG. 8 is a diagram depicting a top view of a second example semiconductor device in which at least one of current or voltage can be applied to substances disposed in liquid, in accordance with one or more example implementations.

FIG. 9 is a diagram depicting a top view of a modified version of a second example semiconductor device in which at least one of current or voltage can be applied to substances disposed in liquid, in accordance with one or more example implementations.

FIG. 10 is a diagram depicting a cross-section of at least a portion of a third example semiconductor device in which at least one of current or voltage can be applied to substances disposed in liquid, in accordance with one or more example implementations.

FIG. 11 is a diagram depicting a process of manufacturing a third example semiconductor device in which at least one of current or voltage can be applied to substances disposed in liquid, in accordance with one or more example implementations.

FIG. 12 is a diagram depicting a top view of a third example semiconductor device in which at least one of current or voltage can be applied to substances disposed in liquid, in accordance with one or more example implementations.

FIG. 13 is a diagram depicting a cross-section of at least a portion of a fourth example semiconductor device in which at least one of current or voltage can be applied to substances disposed in liquid, in accordance with one or more example implementations.

FIG. 14 is a diagram depicting a top view of a fourth example semiconductor device in which at least one of current or voltage can be applied to substances disposed in liquid, in accordance with one or more example implementations.

FIG. 15 is a diagram depicting a cross-section of at least a portion of a fifth example semiconductor device in which at least one of current or voltage can be applied to substances disposed in liquid, in accordance with one or more example implementations.

FIG. 16 is a flow diagram of a process to perform measurements produced in response to electrical stimulation of material disposed in liquids, in accordance with one or more example implementations.

FIG. 17 is a diagram depicting a framework including a fluid delivery device that delivers discrete amounts of liquid, in accordance with one or more example implementations.

FIG. 18 is a diagram of a process to transfer liquid from liquid sources disposed at a first spacing to liquid destinations disposed at a second spacing that is smaller then the first spacing, in accordance with one or more example implementations.

### DETAILED DESCRIPTION

Electrophysiology can include applying electrical stimulation to biological material and measuring changes to electrical properties of the biological material in response to the electrical stimulation to identify one or more characteristics of the biological material. In some cases, electrophysiology can be implemented to determine one or more measures of functionality of the biological material. (e.g., health of tissue or cells) In other scenarios, electrophysiology can be implemented to determine measures of functionality of the biological material in a given environment. For example, the responsiveness of cells to one or more compounds or therapeutics can be measured.

In one or more illustrative examples, patch clamp electrophysiology is a technique that can be used in various fields of medicine and biology for interrogating electrogenic cells such as neurons and cardiomyocytes. Several diseases originate from the electrical properties of these cells, especially diseases of ion channels, or so-called channelopathies comprising neurological disorders, cardiac disorders, pain disorders, and more. Patch clamp enables the study of electrogenic cells by allowing direct control and measurement of cellular membrane potential and ion channel currents. Within fundamental science, patch clamp can be used to understand the basic mechanisms of cell function and disease. Within the study of disease, it can be used to probe specific disease mechanisms and discover molecules that deliver effective treatments.

Manual patch clamp is an approach to perform patch clamp electrophysiology using humancentric equipment and workflows. An example of such an experiment can involve manually bringing a micron-scale glass pipette to the surface of a cell under microscope observation, optionally performing a brief suction through the pipette to disrupt the cell membrane and gain access inside the cell, and then performing current clamp, voltage clamp, dynamic clamp, or other electrical measurement to probe the electrical workings of the cell. Despite skill arguably being the gold standard measurement in many contexts, the technique is very labor intensive and time consuming, involving hours-to-days to perform each measurement.

Automated patch clamp (APC) techniques can help improve the throughput and reduce the cost of performing patch clamp measurements. One class of approaches can be described as direct automation of the manual approach, deploying imaging technology to locate cells within a plate, and robotic technology to automatically manipulate the glass pipette to achieve the seal and measurements. Such techniques can reduce the reliance on a human operator, but do not scale the throughput dramatically.

Another class of approaches can be built around large robotic workstations with liquid handling functions. The robotic head can be used to pipette cells into individual chambers, which can be wells in a well-plate, or customized volumes with additional flow features. Those chambers can feature micron-scale pores that can trap cells under suction and can also open access into cells with additional suction. The electrical measurements can then be performed using a set of discrete electronics, such as multiplexed to electrodes near the chambers. These techniques can dramatically increase the throughput up to 384 independent readouts and can push beyond this.

There is a need for even higher throughput and lower cost. The area of drug discovery for Central-Nervous-System (CNS) disorders has seen extraordinarily low productivity in recent years, leaving many diseases without effective treatments. Most commercial small-molecule screening libraries number in the millions of compounds, and full-file screens with a hundred-site-scale automated patch clamp instrumentation are prohibitive. An increased capacity to screen libraries against electrogenic cells can be a tool to power discoveries, such as in the CNS area.

In one or more implementations, high-throughput patch clamp systems can be manufactured using technologies different from existing techniques. For example, high-throughput patch clamp systems can be manufactured using semiconductor technologies. In this way, the components used to perform patch clamp measurements can be produced on a scale that is smaller than that of existing patch clamp systems. Accordingly, techniques described herein can be used to produce patch clamp systems having a greater number of sites for performing patch clamp measurements than existing techniques and results in higher throughput than existing techniques. In this way, the speed of the development of therapeutic compounds can be increased because more compounds can be tested at a single time than with existing techniques. Because more candidate therapeutic compounds can be tested at a single time, the possibility of identifying viable therapeutic compounds to treat a given biological condition also increases.

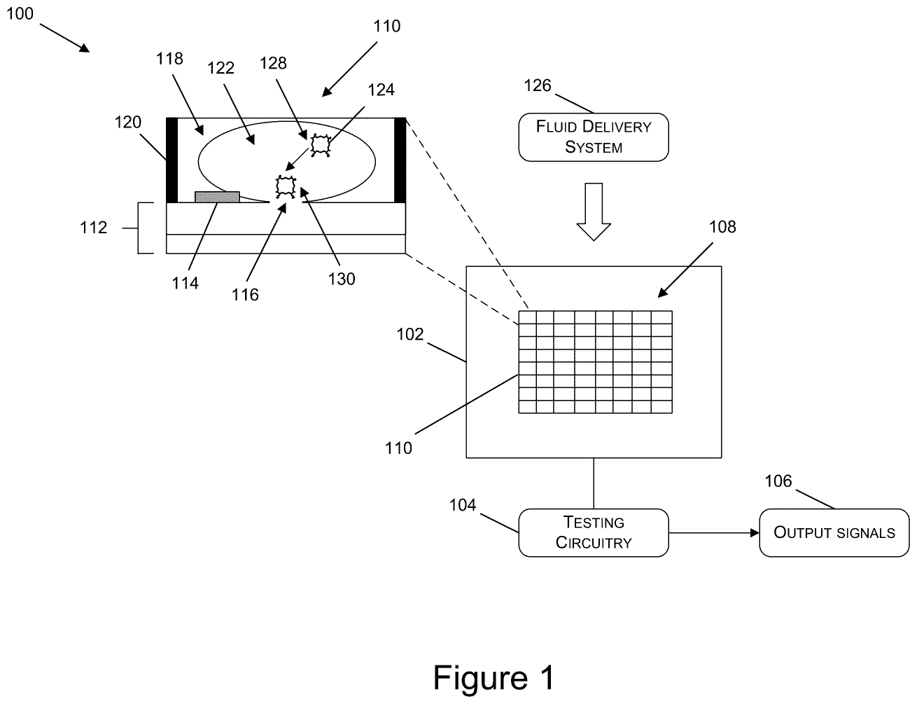

FIG. 1 is a diagram depicting a framework 100 for applying electrical stimulation to substances disposed in fluid using semiconductor devices, in accordance with one or more example implementations. The framework 100 can include a semiconductor device 102. The semiconductor device 102 can be comprised of one or more silicon-containing materials. For example, the semiconductor device 102 can be comprised of at least one of silicon, silicon nitride, or polycrystalline silicon. The semiconductor device 102 can include a die, also referred to herein as a chip, that includes circuitry to apply at least one of voltage or current to liquid disposed in or disposed on the die. The die can also include circuitry to measure signals produced in response to applying at least one voltage or current to the liquid. In various examples, biological material can be disposed in the liquid. In these scenarios, the signals produced in response to applying voltage and/or current to the liquid can indicate one or more characteristics of the biological material.

In one or more examples, the die can include a silicon-containing substrate with circuitry formed on an outer surface of the die. In at least some additional examples, the die can include a silicon-containing substrate with a plurality of layers and circuitry formed on and/or formed within at least a subset of the plurality of layers. The circuitry can be formed according to one or more complementary metal-oxide semiconductor (CMOS) technologies. In one or more illustrative examples, the semiconductor device 102 can include or be coupled to testing circuitry 104. The testing circuitry 104 can cause at least one of voltage or current to be applied to fluid disposed on the semiconductor device 102 and generate output signals 106 in response to the electrical stimulation. In one or more additional illustrative examples, the testing circuitry 104 can perform at least one of a voltage clamp process, a current clamp process, or a dynamic clamp process.

The output signals 106 can correspond to an electrical response of biological material located in the fluid to the voltage and/or current stimuli. In at least some examples, the semiconductor device 102 can store at least a portion of the output signals 106. In one or more additional examples, the semiconductor device 102 can include additional circuitry to analyze the output signals 106. In one or more further examples, the semiconductor device 102 can include hardware processing resources and memory with the hardware processing resources being configured to execute computer-readable instructions to analyze the output signals 106.

In various examples, the testing circuitry 104 can implement one or more optical stimulation protocols. For example, the testing circuitry 104 can cause electromagnetic radiation to be applied to fluid disposed on the semiconductor device 102. In one or more additional examples, an electromagnetic radiation source can be external to the semiconductor device 102. In one or more illustrative examples, the testing circuitry 104 can include one or more optical filters to provide electromagnetic radiation having one or more specified wavelengths to fluid disposed on the semiconductor device 102. The testing circuitry 104 can measure an optical response of fluid and/or substances disposed in fluid located on the semiconductor device 102. In various examples, the testing circuitry 104 can produce output signals 106 that correspond to a fluorescence response to optical stimulation by at least one of fluids or substances disposed in fluids located on the semiconductor device 102.

The die can be disposed in packaging of the semiconductor device 102. The packaging can be comprised of one or more polymeric materials. Additionally, the packaging can be comprised of one or more metallic materials. Further, the packaging can be comprised of one or more ceramic materials. In one or more examples, the packaging can include circuitry coupled to the circuitry of the die. In at least some examples, the semiconductor device 102 can include one or more routing layers to carry electrical signals from circuitry formed on the die to circuitry formed on the packaging.

The semiconductor device 102 can also include circuitry to directly or indirectly couple the die to one or more external computing devices. In these instances, signals generated by circuitry on the die can be sent to the one or more external computing devices. in one or more illustrative examples, the semiconductor device 102 can include circuitry to wirelessly communicate information to one or more external computing devices, such as via a Bluetooth communication protocol or an Institute of Electrical and Electronics Engineers (IEEE) 802.11 communications protocol. In at least some scenarios, the one or more external computing devices can analyze the signals produced by the circuitry on the die to determine one or more characteristics of at least one of fluid or biological materials disposed on the semiconductor device 102.

The semiconductor device 102 can include an array of sites 108. The array of sites 108 can be formed on and/or can be formed by the die of the semiconductor device 102. Individual sites of the array of sites 108 can include components to apply at least one of voltage or current to fluid disposed in the individual site and to measure signals produced in response to the voltage and/or current applied to the fluid. In one or more examples, the sites of the array of sites 108 can be arranged in rows and columns. In various examples, the rows of sites can be in fluid communication with one another via one or more channels formed in the die of the semiconductor device 102. In one or more additional examples, the columns of sites can be in fluid communication with one another via one or more channels formed in the die of the semiconductor device 102. In one or more illustrative examples at least 10 channels, at least 50 channels, at least 100 channels, at least 250 channels, at least 500 channels, at least 1000 channels, at least 200 channels, at least 3000 channels, at least 4000 channels, at least 5000 channels, at least 6000 channels, at least 7000 channels, at least 8000 channels, at least 9000 channels, at least 10,000 channels, or more can be formed in the die of the semiconductor device 102 to couple sites of the array of sites 108. In one or more additional illustrative examples, from 10 channels to 25,000 channels, from 100 channels to 20,000 channels, from 1000 channels to 15,000 channels, from 5000 channels to 10,000 channels, from 1000 channels to 10,000 channels, from 100 channels to 1000 channels, from 10 channels to 100 channels, from 2000 channels to 8000 channels, or from 8000 channels to 12,000 channels can be formed in the dies of the semiconductor device 102 to couple sites of the array of sites 108.

In various examples, the array of sites 108 can include at least 5 sites, at least 20 sites, at least 50 sites, at least 100 sites, at least 200 sites, at least 500 sites, at least 1000 sites, at least 2000 sites, at least 3000 sites, at least 4000 sites, at least 5000 sites, at least 6000 sites, at least 7000 sites, at least 8000 sites, at least 9000 sites, at least 10,000 sites, at least 11,000 sites, at least 12,000 sites, at least 13,000 sites, at least 14,000 sites, at least 15,000 sites, or more. To illustrate, the array of sites 108 can include from 5 sites to 100,000 sites, from 10 sites to 50,000 sites, from 100 sites to 20,000 sites, from 1000 sites to 10,000 sites, from 10,000 sites to 50,000 sites, from 10,000 sites to 30,000 sites, from 5000 sites to 10,000 sites, from 8000 sites to 15,000 sites, from 1000 sites to 5000 sites, from 100 sites to 1000 sites, from 5 sites to 100 sites, from 500 sites to 2000 sites, or from 2000 sites to 8000 sites.

The array of sites 108 can have a rectangular shape, a circular shape, or an ellipsoidal shape. In one or more examples, the array of sites 108 can have a surface area from about 0.1 cm² to about 25 cm², from about 0.5 cm² to about 15 cm², from about 1 cm² to about 10 cm², from about 2 cm² to about 8 cm², from about 3 cm² to about 10 cm², from about 1 cm² to about 5 cm², from about 0.1 cm² to about 1 cm², from about 4 cm² to about 10 cm², or from about 8 cm² to about 15 cm². In illustrative scenarios where the array of sites 108 has a rectangular shape, the array of sites 108 can have a length and a width. In these instances, the array of sites 108 can have at least one of a length or width from about 0.1 cm to about 5 cm, from about 0.5 cm to about 4 cm, from about 1 cm to about 3 cm, from about 2 cm to about 5 cm, or from about 0.5 cm to about 2 cm.

Individual sites 110 of the array of sites 108 can include a number of features. For example, individual sites 110 of the array of sites 108 can include a support structure 112. The support structure 112 can include a number of substrates. In one or more examples, the support structure 112 can include a number of substrates of a die of the semiconductor device 102. In at least some examples, the support structure 112 can include one or more silicon-containing substrates. For example, the support structure 112 can include one or more substrates comprised at least primarily of silicon. In one or more additional examples, the support structure 112 can include one or more substrates comprised of at least one of silicon carbide or silicon nitride. In one or more further examples, the support structure 112 can include one or more substrates comprised of one or more polymeric materials. To illustrate, the support structure 112 can include one or more substrates comprised of one or more polyimides. In various examples, the support structure 112 can include one or more polycrystalline silicon layers. In still other examples, the support structure 112 can include one or more glass substrates. The support structure 112 can also comprise one or more passivation layers. The one or more passivation layers can be comprised of at least one of one or more oxide materials or one or more nitride materials.

Additionally, the support structure 112 can include one or more functional layers. The one or more functional layers can include circuitry to apply at least one of voltage or current to fluid disposed in the individual site 110 and to generate signals in response to the voltage and/or current being applied to the fluid. The support structure 112 can also include one or more bond pads and a number of thin film transistor (TFT) switches to control the flow of current to electrodes formed in the individual sites 110. In the illustrative example of FIG. 1, the individual site 110 includes an electrode 114. In various examples, the individual sites 110 can also include or associated with one or more reference electrodes.

The individual sites 110 can also include a pore 116. The pore 116 can include an opening in one or more layers of the support structure 112. For example, the pore 116 can include an opening in one or more passivation layers of the support structure 112. In one or more additional examples, the pore 116 can include an opening in one or more glass substrates of the support structure 112. In various examples, the pore 116 can provide fluid communication between one or more channels formed in the support structure 112 and a well 118 that is part of the individual site 110. To illustrate, one or more microfluidic channels can be formed in the support structure 112 and the pore 116 provides a means for liquid disposed in the well 118 to access the one or more microfluidic channels formed in the support structure 112. In at least some examples, the pore 116 can be disposed in a vertically oriented direction. In one or more additional examples, the pore 116 can be disposed in a laterally oriented direction. In one or more examples, the well 118 can be formed by a plurality of walls 120. The plurality of walls 120 can be comprised of one or more polymeric materials. In one or more illustrative examples, the plurality of walls 120 can be comprised of a photoresist material.

In various examples, a liquid can be disposed in the well 118. In at least some examples, the liquid disposed in the well 118 can be in the form of a droplet 122. One or more biological cells 124 can be disposed in the droplet 122. The one or more biological cells 124 can include biological material that is undergoing testing and/or experiments using the semiconductor device 102. In one or more illustrative examples, the one or more biological cells 124 can comprise electrogenic cells. In one or more additional illustrative examples, the one or more biological cells 124 can comprise optogenetic cells. In one or more further illustrative examples, the one or more biological cells 124 can include at least one of neurons or cardiomyocytes. In one or more illustrative examples, the wells of the individual sites 110 can be designed to electrically and fluidically isolate the one or more biological cells located in a first site of the array of sites 108 from additional sites included in the array of sites 108 during at least a portion of the process to apply electrical stimulation to the droplet 122 and measure the response to the electrical stimulation. For example, the structure of the sites 110 can cause the biological cells 124 to be at least one of electrically or fluidically isolated from biological cells included in additional sites. Further, materials forming surfaces of the sites 110 can be selected to electrically and fluidically isolate biological cells in one site of the array of sites 108 from other sites included in the array of sites 108. To illustrate, materials of the walls 120 and materials of the support structure 112 that form a floor of the wells 118, can be comprised of materials that result in the biological cells of one site to be at least one of fluidically or electrically isolated from biological cells of additional sites of the array of sites 108.

In some implementations, the walls 120 may not be present in one or more of the sites 110. In these scenarios, the sites 110 can include hydrophobic coatings and/or low surface energy materials on the surface of the support structure 112 that comprises a floor of the site 110 to minimize or prevent fluidic cross-connections between the sites 110. Examples of hydrophobic and/or low surface energy materials to coat the surface of the support structure 112 can include one or more photoresist materials, polytetrafluoroethylene (PTFE), one or more polyimides, or one or more silanes. In one or more illustrative examples, the one or more photoresist materials can include one or more epoxy-based negative photoresist materials, such as SU-8. In one or more additional illustrative examples, the one or more silanes can include octadecyltrimethoxysilane. In various examples, regions of the surface of the support structure 112 that comprise a floor of the site 108 that are not covered by hydrophobic and/or low surface energy materials can include hydrophilic materials. For example, regions of the surface of the support structure 112 around the pore 116 can be coated with one or more hydrophilic materials to result in wetting of the region around the pore 116. Examples of hydrophilic materials used to coat the region around the pore 116 can include at least one of silicon oxide, silicon nitride, titanium oxide, or aluminum oxide.

The droplet 122 can be disposed in the well 118 by a fluid delivery system 126. The fluid delivery system 126 can deliver a volume of liquid to the semiconductor device 102. In one or more examples, the fluid delivery system 126 can deliver discrete amounts of liquid to individual sites 110 of the array of sites 108. In one or more additional examples, the fluid delivery system 126 can supply a volume of liquid to the semiconductor device 102 that is distributed to individual sites 108 using a fluid distribution arrangement of the semiconductor device 102. For example, the semiconductor device 102 can include fluid distribution channels to deliver an amount of liquid to the sites 110. In various examples, the fluid delivery system 126 can deliver liquid to the semiconductor device 102 in one or more batches. For example, the fluid delivery system 126 can provide a discrete volume of liquid to the semiconductor device 102 at a given time. In one or more additional examples, the fluid delivery system 126 can continuously provide liquid to the semiconductor device 102 over a period of time.

In one or more examples, the fluid delivery system 126 can include a number of pipettes that are included in an automated liquid handling system to deliver liquid to at least a portion of the array of sites 108 of the semiconductor device 102. For example, the fluid delivery system 126 can include a robotic liquid handling system to provide liquid to the array of sites 108. In various examples, the semiconductor device 102 can include one or more pipette landing sites to receive liquid from the fluid delivery system 126. Additionally, the fluid delivery system 126 can include a number of pins on which droplets can be disposed. The droplets disposed on the pins can be released and supplied to the array of sites 108. In still other examples, the fluid delivery system 126 can include a substrate with a number of openings. Pressure can be applied to fluid stored in the substrate to cause at least a portion of the fluid to be dispensed to at least a portion of the array of sites 108.

During a cycle of applying at least one of voltage or current to the droplet 122 and measuring an electrical response of the droplet 122, a pressure difference, such as a negative pressure can be applied to move the biological cell 124 from a first position 128 to a second position 130. The second position 130 can correspond to a position of the pore 116. In one or more examples, the pressure difference can be applied to cause a seal to be formed between the biological cell 124 and a surface of the support structure 112 that forms a floor of the well 118. Applying pressure difference to the semiconductor device 102 can also cause a disruption in the membrane of the biological cell 124. The disruption of the membrane of the biological cell 124 can provide access to the internal cellular components. In one or more examples, by causing the internal components of the biological cell 124 to be exposed to one or more substances, ion channels can be formed in the cell membrane of the biological cell 124 in response to electrical stimulation of the biological cell 124 in the presence of one or more additional substance. The ion channels can cause electrical signals to be produced that are measured by the testing circuitry 104.

In one or more illustrative examples, the one or more biological cells 124 can be exposed to one or more small molecules. The one or more small molecules can include candidate molecules for treating one or more biological conditions. In various examples, the one or more biological conditions can include diseases related to the central nervous system. In at least some examples, the small molecules can include candidate treatments for one or more biological conditions related to the human brain. In still other examples, the small molecules can include candidate treatments for one or more biological conditions related to the human heart. Measuring changes in electrical properties of the fluid in response to the one or more biological cells 124 being exposed to the one or more small molecules can indicate a response of the one or more biological cells 124 to treatments of a biological condition that involves the one or more small molecules. In at least some examples, measuring changes in electrical properties of the fluid in response to the one or more biological cells 124 being exposed to the one or more small molecules can indicate an effectiveness of the one or more small molecules in treating a biological condition.

In various examples, oil-in-water implementations can also be implemented. The oil can be used as a separator of between aqueous volumes. The oil can also minimize evaporation of aqueous solutions.

Although the illustrative example of FIG. 1 describes features of a single semiconductor device 102, in one or more additional implementations, multiple semiconductor devices 102 can be disposed on substrate. For example, multiple semiconductor devices 102 can be disposed on a plate. To illustrate, a plate having a number of wells can be provided with a semiconductor device 102 disposed in individual wells. In one or more examples, the plate can have from 6 wells to 1536 wells or more arranged in an array. The plate can be coupled with a fluid delivery substrate having one or more inlet ports to provide liquid to the wells of the plate and one or more outlet ports to drain liquid from the plate. In at least some examples, gaskets can be present to provide enable a seal to be formed between the plate and the fluid delivery substrate. In various examples, patch clamp measurements can be performed in relation to semiconductor devices 102 located in individual wells of the plate. In one or more examples, at least a portion of the individual semiconductor devices 102 can perform patch clamp measurements with respect to liquids that include different therapeutic compounds.

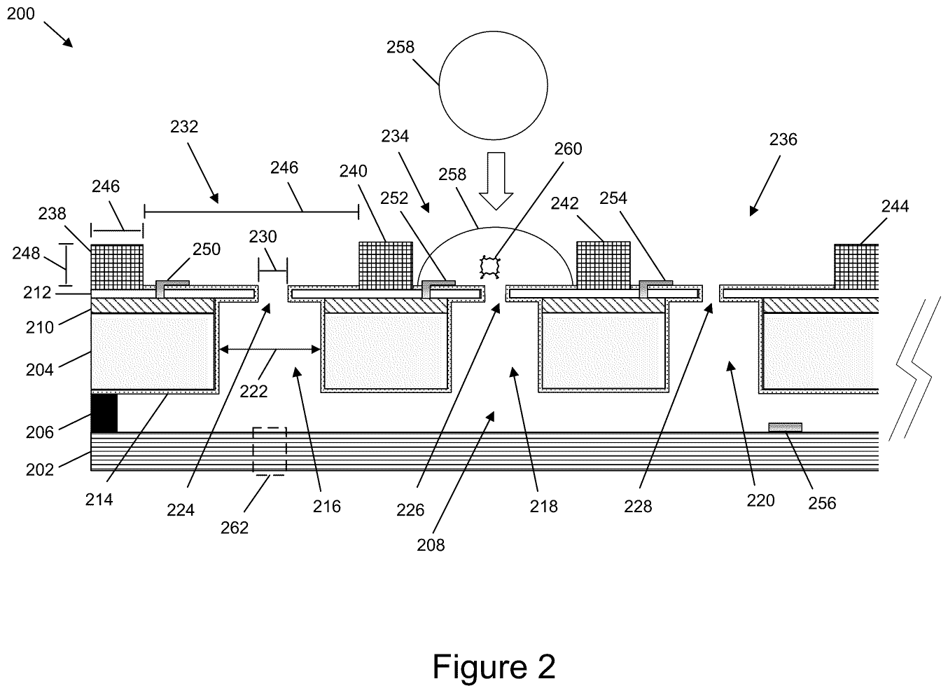

FIG. 2 is a diagram depicting a cross-section of at least a portion of a first example semiconductor device 200 in which at least one of current or voltage can be applied to substances disposed in fluid, in accordance with one or more example implementations. In at least some examples, the first example semiconductor device 200 can correspond to an implementation of the semiconductor device 102 described in relation to FIG. 1.

The semiconductor device 200 can include a substrate 202. The substrate 202 can be comprised of one or more glass materials. The substrate 202 can also be comprised of one or more silicon wafers having one or more passivation layers. The semiconductor device 200 can also include a semiconductor layer 204. The semiconductor layer 204 can include a silicon-containing layer. In one or more examples, the semiconductor layer 204 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

A spacer 206 can be disposed between the substrate 202 and the semiconductor layer 204. In one or more illustrative examples, the spacer 206 can comprise a gasket. In various examples, the spacer 206 can be disposed around a periphery of an array of sites, such as the array of sites 108 described in relation to FIG. 1. A microfluidics region 208 can be formed by the substrate 202, the semiconductor layer 204, and the spacer 206. In one or more examples, the substrate 202 can form a lower boundary of the microfluidics region 208 and the spacer 206 can form one or more side boundaries of the microfluidics region 208.

In addition, the semiconductor device 200 can include a functional layer 210 disposed on the semiconductor layer 204. The functional layer 210 can include circuitry formed on one or more substrates. In one or more examples, the functional layer 210 can include circuitry to apply at least one of voltage or current to liquid disposed on the semiconductor device 200 and to measure signals produced in response to the electrical stimulation provided to the liquid. In one or more illustrative examples, the functional layer 210 can include circuitry to perform at least one of voltage clamp operations, current clamp operations, or dynamic clamp operations. In various examples, the functional layer 210 can include analog-to-digital converters (ADCs), digital-to-analog converters (DACs), amplifiers, digital signal processing circuitry, one or more combinations thereof, and the like. The functional layer 210 can also include routing circuitry to carry electrical signals to locations on a die and/or on packaging of the semiconductor device 200 for further processing. The circuitry of the functional layer 210 can be produced using CMOS technologies.

Further, the semiconductor device 200 can include one or more passivation layers 212 disposed on the functional layer 210. The one or more passivation layers 212 can be comprised of one or more oxide materials and/or one or more nitride materials. For example, the one or more passivation layers 212 can be comprised of at least one of silicon oxide, silicon nitride, aluminum oxide, or titanium oxide. In one or more additional examples, the one or more passivation layers 212 can be comprised of polycrystalline silicon. In various examples, the one or more passivation layers 212 can be comprised of polycrystalline silicon doped with at least one of arsenic, phosphorus, or boron. The one or more passivation layers 212 can also be comprised of undoped polycrystalline silicon. The one or more passivation layers 212 can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

One or more coatings 214 can be disposed over outer surfaces of the semiconductor layer 204, the functional layer 210, and the one or more passivation layers 212. The one or more coatings 214 can include one or more resistive materials. To illustrate, the one or more coatings can include one or more oxide materials or one or more nitride materials. For example, the one or more coatings 214 can comprise at least one of silicon oxide, silicon nitride, or aluminum oxide. The one or more coatings 214 can have a thickness from about 0.01 μm to about 3 μm, from about 0.05 μm to about 2 μm, from about 0.1 μm to about 1.5 μm, from about 0.01 μm to about 0.1 μm, from about 0.01 μm to about 0.05 μm, from about 0.1 μm to about 1 μm, from about 0.05 μm to about 0.5 μm, or from about 1 μm to about 2 μm.

A number of cavities can be formed by the layers of the semiconductor device 200. In one or more examples, the number of cavities can be in fluid communication with the microfluidics region 208. In the illustrative example of FIG. 2, the semiconductor device 200 can include a first cavity 216, a second cavity 218, and a third cavity 220. Side boundaries of the cavities 216, 218, 220 can be formed by locations of the semiconductor layer 204, the functional layer 210, the one or more passivation layers 212, and the one or more coatings 214. Although the illustrative example of FIG. 2 shows three cavities of the semiconductor device 200, the semiconductor device 200 can include a greater number of cavities. To illustrate, the semiconductor device 200 can include at least 10 cavities, at least 50 cavities, at least 100 cavities, at least 500 cavities, at least 1000 cavities, at least 2500 cavities, at least 5000 cavities, at least 10,000 cavities, or more. In various examples, the cavities 216, 218, 220 can form channels within the semiconductor device 200 in which one or more liquids can be disposed. The cavities 216, 218, 220 can have a width 222. The width 222 of the cavities 216, 218, 220 can be from 25 μm to about 400 μm, from about 50 μm to about 250 μm, from about 100 μm to about 200 μm, from about 25 μm to about 125 μm, from about 50 μm to about 150 μm, from about 50 μm to about 100 μm, or from about 100 μm to about 150 μm. In one or more illustrative examples, the cavities 216, 218, 220 can have a cylindrical shape. In these scenarios, the width 222 of the cavities 216, 218, 220 can correspond to a diameter.

The semiconductor device 200 can include a number of pores that are formed by openings in the one or more passivation layers 212 and the one or more coatings 214. In the illustrative example of FIG. 2, the semiconductor device 200 can include a first pore 224, a second pore 226, and third pore 228. Although the illustrative example of FIG. 2 shows three pores of the semiconductor device 200, the semiconductor device 200 can include a greater number of pores. For example, the semiconductor device 200 can include at least 100 pores, at least 500 pores, at least 1000 pores, at least 5000 pores, at least 10,000 pores, at least 25,000 pores, at least 50,000 pores, at least 100,000 pores, or more. The pores 224, 226, 228 can have a width 230 from about 1 nanometer (nm) to about 40 μm, from about 10 nm to about 30 μm, from about 0.1 μm to about 25 μm, from about 1 μm to about 20 μm, from about 5 μm to about 15 μm, from about 1 μm to about 10 μm, from about 10 μm to about 20 μm, from about 1 μm to about 5 μm, from about 0.1 μm to about 1 μm, from about 0.5 μm to about 2 μm, or from about 5 μm to about 10 μm.

Further, a spacing between the pores 224, 226, 228 can comprise a pitch of the semiconductor device 200. In one or more examples, the pitch of the semiconductor device 200 can be measured from a center of the first pore 224 to a center of the second pore 226. The pitch of the semiconductor device 200 can also be measured from a center of the second pore 226 to a center of the third pore 228. In at least some examples, the distance from a center of the first pore 224 to a center of the second pore 226 can be at least substantially the same as the distance from a center of the second pore 226 to a center of the third pore 228. In one or more illustrative examples, a pitch of the semiconductor device 200 can be from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

The semiconductor device 200 can also include a number of sites. The number of sites can be structured to apply electrical stimulation to a discrete amount of liquid located in individual sites. In the illustrative example of FIG. 2, the semiconductor device 200 includes a first site 232, a second site 234, and a third site 236. Although the illustrative example of FIG. 2 shows three sites of the semiconductor device 200, the semiconductor device 200 can include a greater number of sites. For example, the semiconductor device 200 can include at least 100 sites, at least 500 sites, at least 1000 sites, at least 5000 sites, at least 10,000 sites, at least 25,000 sites, at least 50,000 sites, at least 100,000 sites, or more.

The sites 232, 234, 236 can be defined by walls. The first site 232 can be enclosed by at least a first wall 238 and a second wall 240. In addition, the second site 234 can be enclosed by at least the second wall 240 and a third wall 242. Further, the third site 236 can be enclosed by the third wall 242 and a fourth wall 244. Although not shown in the illustrative cross sectional view of FIG. 2, the sites 232, 234, 236 can also be defined by walls disposed in rows perpendicular to the walls 238, 240, 242, 244. In this way, the sites 232, 234, 236 can be defined by a rectangular shape having boundaries formed by four walls. The sites 232, 234, 236 can have an inner width 246 from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

In one or more examples, the walls 238, 240, 242, 244 can be comprised of one or more polymeric materials. In at least some examples, the walls 238, 240, 242, 244 can be comprised of one or more photoresist materials. Additionally, the walls 238, 240, 242, 244 can be comprised of one or more hydrophobic materials and/or one or more low surface energy materials. In one or more illustrative examples, the walls 238, 240, 242, 244 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In one or more illustrative examples, the walls 238, 240, 242, 244 can have a height 248 from about 1 nm to about 500 μm, from about 10 nm to about 400 μm, from about 100 nm to about 300 μm, from about 1 μm to about 200 μm, from about 5 μm to about 100 μm, from about 10 μm to about 50 μm, from about 50 μm to about 100 μm, from about 1 μm to about 10 μm, from about 10 μm to about 25 μm, from about 50 μm to about 150 μm, or from about 100 μm to about 200 μm. In one or more additional illustrative examples, the walls 238, 240, 242, 244 can have a thickness 246 from about 0.1 μm to about 50 μm, from about 1 μm to about 30 μm, from about 5 μm to about 20 μm, from about 0.1 μm to about 1 μm, from about 0.5 μm to about 2 μm, from about 1 μm to about 5 μm, from about 1 μm to about 10 μm, from about 5 μm to about 15 μm, or from about 5 μm to about 10 μm.

The individual sites 232, 234, 236 can each include an electrode. The electrodes included in the individual sites 232, 234, 236 can be electrically connected to circuitry of the functional layer 210. In one or more examples, the electrodes can be electrically connected to circuitry of the functional layer 210 by vias formed in the one or more coatings 214, the one or more passivation layers 212, and at least a portion of the functional layer 210. In the illustrative example of FIG. 2, a first electrode 250 can be located in the first site 232, a second electrode 252 can be located in the second site 234, and a third electrode 254 can be located in the third site 236. In one or more examples, the first electrode 250, the second electrode 252, and the third electrode 254 can be used to apply at least one of voltage or current to liquid disposed in the respective sites 232, 234, 236. Additionally, at least one of the first electrode 250, the second electrode 252, and the third electrode 254 can be used to measure signals produced in response to electrical stimulation being provided to liquid disposed in the respective sites 232, 234, 236. The electrodes 250, 252, 254 can be comprised of one or more metallic materials. To illustrative, the electrodes 250, 252, 254 can be comprised of at least one of sliver chloride, platinum, alloys of platinum, gold, or alloys of gold.

In various examples, the semiconductor device 200 can also include at least one reference electrode 256. The at least one reference electrode 256 can be disposed on a surface of the substrate 202 and located in the microfluidics region 208. The at least one reference electrode 256 can be comprised of at least one of sliver chloride, platinum, alloys of platinum, gold, or alloys of gold. Although the at least one reference electrode 256 is shown in the microfluidics region 208 in the illustrative example of FIG. 2, in one or more additional implementations, the at least one reference electrode 256 can be disposed in the semiconductor layer 204 or can include a wire inserted in the microfluidics regions 208.

In one or more examples, liquid can be disposed in the sites 232, 234, 236. For example, the walls 238, 240, 242, 244 and the coated passivation layer(s) can comprise wells that can store a volume of liquid. For example, the walls 238, 240, 242, 244 can form side surfaces of the wells and the coated passivation layer(s) can comprise a floor of the wells. In one or more illustrative examples, volumes of liquid disposed in the individual sites 232, 234, 236 can include from about 1 picoliter (pL) to about 100 nanoliters (nL), from about 5 pL to about 10 nL, from about 10 pL to about 1 nL from about 1 pL to about 100 pL, from about 1 pL to about 50 pL, from about 1 pL to about 10 pL, from about 10 pL to about 500 pL, from about 10 pL to about 100 pL, from about 10 pL to about 50 pL, from about 50 pL to about 500 pL, from about 50 pL to about 250 pL, from about 100 pL to about 1 nL, from about 100 pL to about 500 pL, from about 500 pL to about 5 nL, from about 500 pL to about 2 nL, from about 500 pL to about 1 nL, from about 1 nL to about 100 nL, from about 1 nL to about 50 nL, or from about 1 nL to about 10 nL.

In the illustrative example of FIG. 2, an amount of liquid 258 can be dispensed into the second site 234. Although the illustrative example of FIG. 2 shows the amount of liquid 258 being dispensed into the second site 234, additional amounts of the liquid can also be dispensed into the first site 232, the third site 236, and other sites of the semiconductor device 200. In at least some examples, the amount of liquid 258 can comprise an aqueous solution having one or more biological cells, such as the example biological cell 260. In various examples, the amount of liquid 258 can form a droplet in the second site 234. For example, in scenarios where the walls 240, 242 are comprised of hydrophobic materials and the coating 214 is comprised of a hydrophilic material, the amount of liquid 258 can bead into droplets on the hydrophilic portions of the site and move away from the walls 240, 242. In this way, the droplet of the amount of liquid 258 in the second site can be electrically and fluidically isolated from droplets of liquid disposed in other sites of the semiconductor device 200. Accordingly, independent electrophysiological measurements can be produced for individual sites of the semiconductor device 200.

In one or more illustrative examples, first fluid can be disposed external to the cavities 216, 218, 220 and within the sites 232, 234, 236 and second fluid can be disposed within the cavities 216, 218, 220 as well as in the microfluidics region 208. The first fluid disposed external to the cavities 216, 218 220 and within the sites 232, 234, 236 can correspond to the amount of liquid 258 and can include biological cells, one or more therapeutic compounds, one or more additional reagents, one or more combinations thereof, and the like. In at least some examples, the first fluid can include at least one of NaCl, KCl, HEPES buffer, MgCl₂, CaCl₂, glucose, or ligands. In one or more additional examples, the second fluid disposed within the cavities 216, 218, 220 and within the microfluidics region 208 can include reagents that are used in relation to patch clamp processes and can include, in at least some examples, one or more therapeutic compounds. In one or more implementations, the substrate 202 can include one or more ports 262 that can function to enable fluid to enter and/or leave the microfluidics region 208. In one or more additional examples, an immiscible fluid, such as oil, can be disposed above an aqueous liquid layer that includes the amount of liquid 258 to minimize evaporation of the amount of liquid 258. The immiscible liquid can also serve as a separator of one or more volumes of liquid including biological cells.

In various examples, obtaining patch clamp measurements can include applying a pressure difference between the first fluid disposed in the sites 232, 234, 236 and the second fluid disposed in the cavities 216, 218, 220 and the microfluidics region 208 to move biological cells 260 toward the respective pores 224, 226, 228. In one or more examples, the biological cells 260 can form a seal with the pores 224, 226, 228 such that the biological cells 260 are disposed at least partially within the pores 224, 226, 228 and on the respective floors of the sites 232, 234, 236 comprised of the coated passivation layer(s) 212. In at least some examples, a first pressure difference can be applied to move the biological cells 260 to form seals with the pores 224, 226, 228 and a second pressure difference can be applied to cause rupturing of the membranes of the biological cells 260. In one or more illustrative examples, the pressure difference can correspond to a negative pressure. In still other examples, a single pressure difference can be applied to move the biological cells 260 toward the respective pores 224, 226, 228 and to disrupt the membranes of the biological cells 260.

While the biological cells 260 are pressed against the floor of the respective sites 232, 234, 236 using the second amount of pressure to disrupt the membranes of the biological cells 260, a potential difference can be applied between the electrodes 250, 252, 254 and the reference electrode 256. In these scenarios, voltage clamp measurements can be collected. In one or more additional examples, while the biological cells 260 are pressed against the floor of the respective sites 232, 234, 236 using the second amount of pressure to disrupt the membranes of the biological cells 260, the electrodes 250, 252, 254 can deliver an amount of current to the biological cells 260 through the disrupted membranes of the biological cells 260 to collect current clamp measurements. The measurements collected during the voltage clamp and/or current clamp operations can be used to determine activity of biological cells 260 with respect to one or more therapeutic compounds and/or to one or more experimental conditions. In various examples, the amount of activity of the biological cells 260 during patch clamp operations can indicate a responsiveness of the biological cells 260 to at least one of the one or more therapeutic compounds or the one or more experimental conditions.

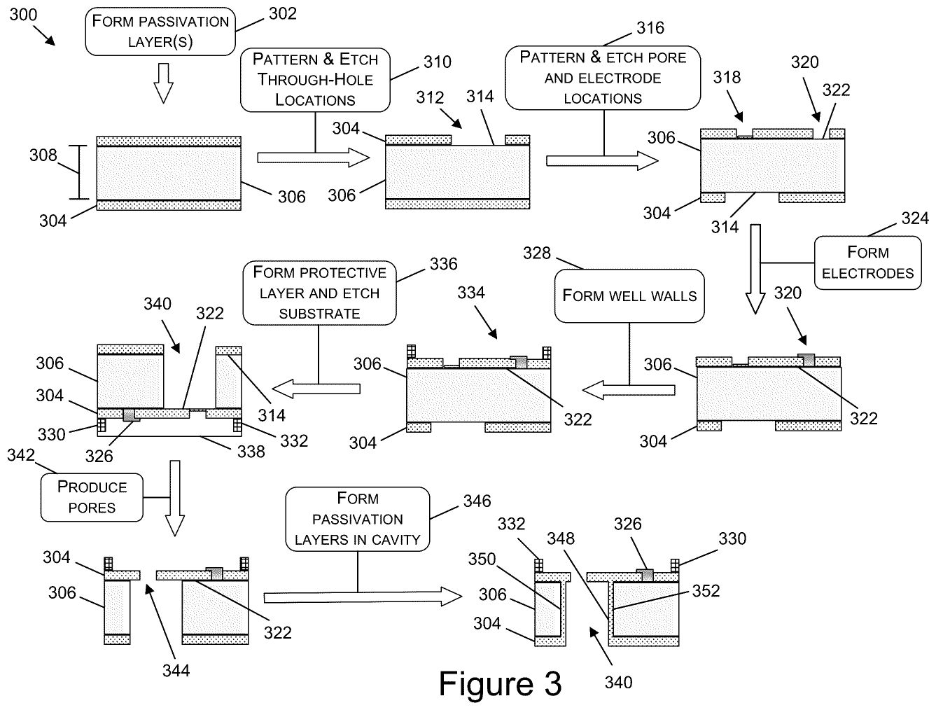

FIG. 3 is a diagram depicting a process 300 of manufacturing a first example semiconductor device in which at least one of current or voltage can be applied to substances disposed in fluid, in accordance with one or more example implementations. In one or more illustrative examples, the process 300 can be implemented to produce semiconductor devices having one or more features of the semiconductor device 200 described in relation to FIG. 1.

The process 300 can include, at 302, forming one or more passivation layers 304 on a semiconductor substrate 306. The semiconductor substrate 306 can be comprised of one or more silicon-containing materials. For example, the semiconductor substrate 306 can include silicon, silicon carbide, or silicon nitride. In one or more illustrative examples, the semiconductor substrate 306 can be comprised of at least about 80% silicon, at least about 90% silicon, at least about 95% silicon, at least about 99% silicon, or at least about 99.5% silicon. In various examples, the semiconductor substrate 306 can have circuitry formed on and/or within the semiconductor substrate 306. The circuitry can include electrical components to apply at least one of voltage or current to liquid. In at least some examples, the semiconductor substrate 306 can include circuitry to perform patch clamp operations.

The semiconductor substrate 306 can have a thickness 308 that is from about 100 μm to about 800 μm, from about 200 μm to about 700 μm, from about 300 μm to about 600 μm, from about 100 μm to about 300 μm, from about 200 μm to about 400 μm, from about 300 μm to about 500 μm, from about 400 μm to about 600 μm, from about 500 μm to about 700 μm, or from about 600 μm to about 800 μm. In one or more examples, the semiconductor substrate 306 can have a first thickness and one or more grinding and/or polishing operations can be performed to reduce the thickness of the semiconductor substrate 306 to a second thickness before the one or more passivation layers are formed on the semiconductor substrate 306.

The one or more passivation layers 304 can be comprised of at least one of one or more resistive materials. For example, the one or more passivation layers 304 can be comprised of at least one of one or more oxides or one or more nitrides. To illustrate, the one or more passivation layers 304 can be comprised of at least one of silicon oxide, silicon nitride, aluminum oxide, or titanium oxide. In scenarios where the one or more passivation layers 304 include an oxide material, the one or more oxide-containing passivation layers can be formed by at least one of thermal oxidation of the semiconductor substrate, chemical vapor deposition, physical vapor deposition, plasma enhanced chemical vapor deposition, thermal evaporation, or electron beam evaporation. Additionally, in situations where the one or more passivation layers 304 include a nitride material, the one or more nitride-containing passivation layers can be formed by at least one of low pressure chemical vapor deposition, plasma enhanced chemical vapor deposition, thermal atomic layer deposition, or plasma enhanced atomic layer deposition.

In one or more illustrative examples, the one or more passivation layers 304 can be formed by performing a first process to form a first passivation layer comprising an oxide-containing material on the semiconductor substrate 306. For example, a thermal process can be performed to form a first silicon oxide layer on the semiconductor substrate 306. The first silicon oxide layer can have a thickness from about 0.1 μm to about 5 μm, from about 0.5 μm to about 4 μm, from about 1 μm to about 3 μm, from about 0.1 μm to about 1 μm, from about 1 μm to about 2 μm, or from about 2 μm to about 3 μm.

In addition, the one or more passivation layers 304 can be formed by performing a second process to form a second passivation layer on the first passivation layer. To illustrate, a nitride-containing passivation layer can be formed on the first silicon oxide layer. In one or more additional illustrative examples, a silicon nitride layer can be formed on the first silicon oxide layer using a low pressure chemical vapor deposition process. The nitride-containing passivation layer can have a thickness from about 0.5 μm to about 10 μm, from about 1 μm to about 8 μm, from about 2 μm to about 6 μm, from about 3 μm to about 5 μm, from about 1 μm to about 5 μm, from about 5 μm to about 10 μm, from about 1 μm to about 3 μm, from about 2 μm to about 4 μm, or from about 3 μm to about 5 μm.

Further, the one or more passivation layers 304 can be formed by performing a third process to form a third passivation layer on the second passivation layer. To illustrate, a second oxide-containing passivation layer can be formed on the nitride-containing passivation layer. In one or more further illustrative examples, a silicon oxide layer, a titanium oxide layer, or an aluminum oxide layer can be formed on the silicon nitride layer using a chemical vapor deposition process, a physical vapor deposition process, a plasma enhanced chemical vapor deposition process, a thermal evaporation process, or an electron beam evaporation process. The second oxide-containing passivation layer can have a thickness from about 10 nm to about 1 μm, from about 50 nm to about 800 nm, from about 100 nm to about 500 nm, from about 500 nm to about 1 μm, from about 50 nm to about 200 nm, from about 100 nm to about 300 nm, from about 200 nm to about 400 nm, or from about 300 nm to about 500 nm.

In various examples, the second oxide-containing passivation layer can function as a stop for one or more subsequent etching processes. Additionally, the first oxide-containing passivation layer can comprise a hydrophilic and biocompatible material. In this way, the first oxide-containing passivation layer can provide adhesion of one or more aqueous liquids to the surface of the first oxide-containing passivation layer and be conducive for a seal being formed with biological cells. In at least some examples, the one or more passivation layers 304 can be formed to provide mechanically robust membranes and produce a low stress film that facilitates patch clamp measurements.

In one or more additional examples, the one or more passivation layers 304 can be comprised of a polycrystalline silicon. In instances where the one or more passivation layers include a polycrystalline material, the polycrystalline silicon-containing passivation layers can be formed using at least one of plasma enhanced chemical vapor deposition or low pressure chemical vapor deposition. In various examples, the polycrystalline silicon layer can be coated with at least one of one or more oxide-containing materials or one or more nitride-containing materials. For example, the one or more passivation layers 304 can include a polycrystalline silicon layer coated with at least one of silicon oxide, silicon nitride, aluminum oxide, or titanium oxide.

At 310, the process 300 can include patterning and etching through-holes locations 312 on a first surface 314 of the semiconductor substrate 306. The through-hole locations 312 can correspond to locations of cavities formed in the semiconductor substrate 306. In various examples, the patterning and etching of the through-hole locations 312 can include removing portions of the one or more passivation layers 304 according to a pattern that corresponds to the through-hole locations 312. In at least some examples, the portions of the one or more passivation layers 304 can be removed to cause portions of the first surface 314 of the semiconductor substrate 306 to be exposed that correspond to the through-hole locations 312.

One or more lithographic processes can be used to pattern and etch the one or more passivation layers 304. For example, a photoresist layer or film can be formed on the one or more passivation layers 304 according to a pattern and then exposed to electromagnetic radiation. In scenarios where the photoresist is a positive photoresist, the through-hole locations are exposed to electromagnetic radiation and the exposed portions of the positive photoresist are removed by the developer solution. In situations where the photoresist is a negative photoresist, the through-hole locations are not exposed to electromagnetic radiation and the unexposed portions of the negative photoresist are removed by the developer solution. In this way, the photoresist pattern forms an etch mask for subsequent etching processes where the through-holes will be formed.

The process 300 can also include, at 316, patterning and etching pore locations 318 and electrode locations 320. The patterning and etching performed at 316 can be performed in relation to a second surface 322 of the semiconductor substrate 306. The second surface 322 of the semiconductor substrate 306 can be disposed at least substantially parallel with respect to the first surface 314 of the semiconductor substrate 306. The pore locations 318 can correspond to locations in the one or more passivation layers 304 where pores of the sites of semiconductor devices are to be formed. In one or more examples, a portion of the one or more passivation layers 304 remains after the patterning and etching performed at 316 in relation to the pore locations 318. For example, a passivation layer can remain on the second surface 322 of the semiconductor substrate 306 at the pore locations 318 after additional passivation layers are removed as part of the patterning and etching performed at 316. In one or more illustrative examples, an oxide-containing passivation layer can remain on the second surface 322 of the semiconductor substrate 306 at the pore locations 318 and at least one nitride-containing passivation layer and/or at least one additional oxide-containing passivation layer can be removed after the patterning and etching performed at 316.

The electrode locations 320 can correspond to locations in the one or more passivation layers 304 where electrodes of the sites of semiconductor devices are to be formed. In one or more examples, the one or more passivation layers 304 are removed at the electrode locations 320 to cause portions of the second surface 322 of the semiconductor substrate 306 to be exposed after the patterning and etching is performed at 316 in relation to the electrode locations 320. For example, at least one nitride-containing passivation layer and/or at least one oxide-containing passivation layer can be removed as part of the patterning and etching performed at 314 in relation to the electrode locations 320. Although not shown in the illustrative example of FIG. 3, in at least some cases, patterning and etching the electrode locations 320 can include forming vias within the semiconductor substrate 306. The vias can connect the electrodes formed on the second surface 322 of the semiconductor substrate 306 with circuitry included in a functional layer of the semiconductor substrate 306.

At 324, the process 300 can include forming electrodes 326 on the second surface 322 of the semiconductor substrate 306 at the electrode locations 320. The electrodes 326 can be formed in the electrode locations 320 by at least one of electroplating, sputter deposition, chemical vapor deposition, or physical vapor deposition. The electrodes 326 can be comprised of one or more metals that can be used to pass current through an aqueous liquid. In one or more examples, the electrodes 326 can be comprised of at least one of silver chloride, platinum, alloys of platinum, gold, or alloys of gold. In implementations where vias are formed within the semiconductor substrate 306 as part of the electrodes 326, the metallic material disposed in the vias can be the same as or different from the materials used to form the portion of the electrodes 326 disposed on the second surface 322 of the semiconductor substrate 306. To illustrate, the one or more metallic materials disposed on the vias can include at least one of gold, alloys of gold, copper, or alloys of copper. Forming one or more metallic materials in the vias can be performed using at least one of one or more electroplating processes, one or more chemical vapor deposition processes, or one or more physical vapor deposition processes.

Additionally, the process 300 can include, at 328, forming walls of the sites of a semiconductor device that can be used to perform patch clamp measurements. For example, a first wall 330 and a second wall 332 can be formed on the one or more passivation layers 304 disposed on second surface 322 of the semiconductor substrate 306. In one or more examples, the first wall 330 and the second wall 332 can form a well 334 in which liquid can be disposed. The walls 330, 332 can separate the well 334 from other wells of the semiconductor device. In various examples, the walls 330, 332 can be formed from one or more hydrophobic materials. To illustrate, the walls 330, 332 can be formed from one or more photoresist materials. In one or more illustrative examples, the walls 330, 332 can be formed from at least one of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In at least some examples, the walls 330, 332 can be formed using one or more lithographic processes to pattern one or more photoresist materials according to the locations of the walls 330, 332.

Further, the process 300 can include, at 336, forming a protective layer 338 over features disposed on the second surface 322 of the semiconductor substrate 306 and etching the first surface 314e of the semiconductor substrate 306 at the through-hole locations 312 to form cavities 340. In at least some examples, the semiconductor device can be flipped before the operations are performed at 336. In one or more examples, the protective layer 338 can be formed over the one or more passivation layers 304 formed on the second surface 322 of the semiconductor substrate 306, the first wall 330, the second wall 332, and the electrode 326. The protective layer 338 can be comprised of one or more polymeric materials. For example, the protective layer 338 can be comprised of polymethylmethacrylate, polycarbonate, or one or more photoresist materials.

After the protective layer 338 is formed on the components disposed on the second surface 322 of the semiconductor substrate 306, portions of the semiconductor substrate 306 that correspond to the through-hole locations 312 can be removed by one or more etch processes to produce the cavities 340. The one or more etch processes used to produce the cavities 340 within the semiconductor substrate 306 can include one or more deep anisotropic etch processes. For example, a deep reactive ion etching process can be used to remove portions of the semiconductor substrate 306 to form the cavities 340. In one or more additional examples, one or more wet etching processed can be performed to remove portions of the semiconductor substrate 306 to form the cavities 340. To illustrate, a potassium hydroxide etching process or a tetramethylammonium hydroxide (TMAH) etching process can be performed to remove portions of the semiconductor substrate 306 to form the cavities 340.

At 342, the process 300 can include producing pores 344 in the one or more passivation layers 304 disposed on the second surface 322 of the semiconductor substrate 306. In one or more examples, the semiconductor device can be flipped before performing operations at 342. To illustrate, remaining portions of the one or more passivation layers 304 disposed at the pore locations 318 can be removed to form the pores 344. For example, one or more oxide-containing passivation layers 304 disposed at the pore locations 318 can be removed to form the pores 344. The pores 344 can be produced by at least one of plasma etching, wet etching, or ion beam etching of the remaining portions of the one or more passivation layers 304 disposed at the pore locations 318. In one or more illustrative examples, a buffered oxide etch can be performed to remove the remaining portions of the one or more passivation layers 304 disposed at the pore locations 318 to form the pores 344. In various examples, the protective layer 338 can be removed before the pores 344 are formed.

In addition, the process 300 can include, at 346, forming one or more additional passivation layers 348 within the cavities 342. For example, one or more additional passivation layers 348 can be formed on a first side wall 350 and a second side wall 352 of the cavity 340. In one or more examples, the one or more additional passivation layers 348 can be comprised of one or more oxide-containing materials. To illustrate, the one or more additional passivation layers 348 can be comprised of silicon oxide. The one or more additional passivation layers 348 can be formed from at least one of atomic layer deposition or plasma enhanced chemical vapor deposition. In one or more illustrative examples, the one or more additional passivation layers 348 can have a thickness from about 10 nm to about 1 μm, from about 50 nm to about 800 nm, from about 100 nm to about 500 nm, from about 500 nm to about 1 μm, from about 50 nm to about 200 nm, from about 100 nm to about 300 nm, from about 200 nm to about 400 nm, or from about 300 nm to about 500 nm.

In at least some examples, the formation of the one or more additional passivation layers on the side walls 350, 352 of the cavity 340 can also cause formation of the one or more additional passivation layers on additional surfaces of the semiconductor device, such as surfaces of the electrode 326, surfaces of the walls 330, 332, and/or surfaces of the one or more passivation layers 304. In these situations, portions of the one or more additional passivation layers 348 can be removed from the additional surfaces of the semiconductor device. For example, one or more etching processes can be performed to remove one or more additional oxide layers from the electrodes 326.

Although the illustrative example of FIG. 3 describes the formation of features of a single site of a semiconductor device, the process 300 can be used to form features of multiple sites of a semiconductor device. For example, the process 300 can be used to form features of at least 5 sites, at least 20 sites, at least 50 sites, at least 100 sites, at least 200 sites, at least 500 sites, at least 1000 sites, at least 2000 sites, at least 5000 sites, at least 8000 sites, at least 10,000 sites, at least 25,000 sites, at least 50,000 sites, at least 75,000 sites, at least 100,000 sites, or more.

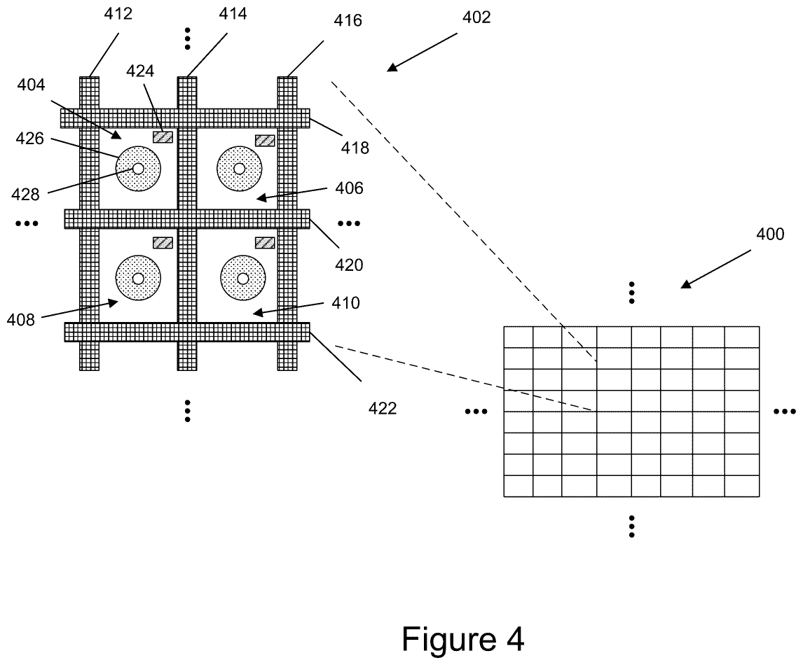

FIG. 4 is a diagram depicting a top view of a first example semiconductor device having an array of sites 400 in which at least one of current or voltage can be applied to substances disposed in fluid, in accordance with one or more example implementations. In one or more examples, the array of sites 400 can include sites that correspond to sites 232, 234, 236 described in relation to FIG. 2 and a site manufactured according to the process 300 described in relation to FIG. 3. In one or more examples, the array of sites 400 can have tens of sites, hundreds of sites, thousands of sites, up to tens of thousands of sites, or more.

FIG. 4 indicates a top view of a subsection 402 of the array of sites 400. The subsection includes a number of individual sites and a number of features of the individual sites. For example, the subsection 402 includes a first site 404, a second site 406, a third site 408, and a fourth site 410. The boundaries of the sites 404, 406, 408, 410 can be formed by rows and columns of walls comprised of one or more polymeric materials. For example, the boundaries of the sites 404, 406, 408, 410 can be formed by a first wall section 412 comprised of one or more polymeric materials, a second wall section 414 comprised of the one or more polymeric materials, and a third wall section 416 comprised of the one or more polymeric materials. Additionally, the boundaries of the sites 404, 406, 408, 410 can be formed by a fourth wall section 418 comprised of the one or more polymeric materials, a fifth wall section 420 comprised of the one or more polymeric materials, and a sixth wall section 422 comprised of the one or more polymeric materials. The first wall section 412, the second wall section 414, and the third wall section 416 can be disposed at least substantially perpendicular with respect to the fourth wall section 418, the fifth wall section 420, and the sixth wall section 422.

Individual sites 404, 406, 408, 410 can include an electrode 424. Additionally, the individual sites 404, 406, 408, 410 can include a membrane 426. The membrane 426 can be comprised of one or more hydrophilic materials. In one or more examples, the one or more hydrophilic materials can include one or more oxide-containing materials that comprise at least one passivation layer formed over one or more layers of a semiconductor substrate of the semiconductor device. Although the shape of the membrane 426 shown in the illustrative example of FIG. 4 is circular, the membrane 426 can have other shapes, such as rectangular shapes, ellipsoidal shapes, or irregular shapes. Further, the individual sites 404, 406, 408 410 can include a pore 428 that is disposed in the membrane.

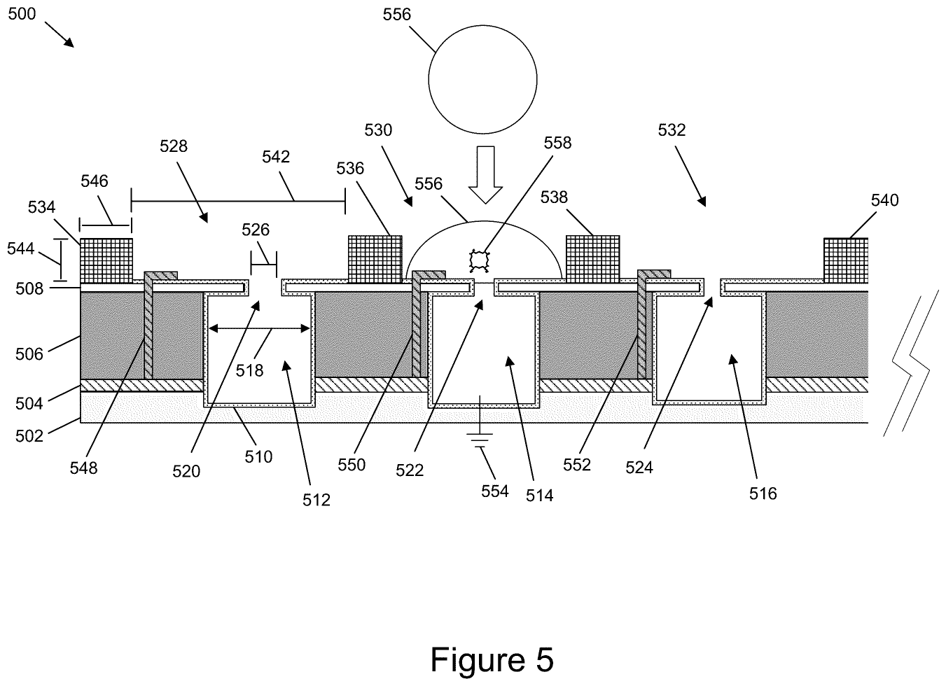

FIG. 5 is a diagram depicting a cross-section of at least a portion of a second example semiconductor device 500 in which at least one of current or voltage can be applied to substances disposed in fluid, in accordance with one or more example implementations. In at least some examples, the second example semiconductor device 500 can correspond to an implementation of the semiconductor device 102 described in relation to FIG. 1.

The semiconductor device 500 can include a semiconductor layer 502. The semiconductor layer 502 can include a silicon-containing layer. In one or more examples, the semiconductor layer 502 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

In addition, the semiconductor device 500 can include a functional layer 504 disposed on the semiconductor layer 502. The functional layer 504 can include circuitry formed on one or more substrates. In one or more examples, the functional layer 504 can include circuitry to apply at least one of voltage or current to liquid disposed on the semiconductor device 500 and to measure signals produced in response to the electrical stimulation provided to the liquid. In one or more illustrative examples, the functional layer 504 can include circuitry to perform at least one of voltage clamp operations, current clamp operations, or dynamic clamp operations. In various examples, the functional layer 504 can include analog-to-digital converters (ADCs), digital-to-analog converters (DACs), amplifiers, digital signal processing circuitry, one or more combinations thereof, and the like. The functional layer 504 can also include routing circuitry to carry electrical signals to locations on a die and/or on packaging of the semiconductor device 500 for further processing. The circuitry of the functional layer 504 can be produced using CMOS technologies.

The semiconductor device 500 can also include a silicon-on-insulator substrate 506. In one or more illustrative examples, the silicon-on-insulator substrate 506 can correspond to a micro-electro-mechanical systems (MEMS) substrate. The silicon-on-insulator substrate 506 can include one or more sublayers. For example, the silicon-on insulator substrate 506 can include an integrated circuit layer. The integrated circuit layer can include a silicon-containing layer. In at least some examples, circuitry can be disposed on the integrated circuit layer. In one or more examples, the integrated circuit layer can also be referred to herein as a device layer. The integrated circuit layer can have a thickness from about 10 μm to about 500 μm, from about 20 μm to about 400 μm, from about 30 μm to about 300 μm, from about 40 μm to about 200 μm, from about 20 μm to about 100 μm, from about 100 μm to about 200 μm, from about 50 μm to about 150 μm, from about 150 μm to about 250 μm, from about 200 μm to about 300 μm, from about 250 μm to about 350 μm, or from about 300 μm to about 400 μm,

Additionally, the silicon-on-insulator substrate 506 can include an insulator layer disposed under the integrated circuit layer. In one or more examples, the insulator layer can be comprised of an oxide-containing material. For example, the insulator layer can be comprised of silicon dioxide. In one or more additional examples, the insulator layer can be comprised of a sapphire-containing materials. In various examples, the insulator layer can be referred to herein as a buried oxide layer. The insulator layer can have a thickness from about 0.1 μm to about 25 μm, from about 0.5 μm to about 20 μm, from about 1 μm to about 15 μm, from about 2 μm to about 10 μm, from about 1 μm to about 5 μm, from about 5 μm to about 10 μm, from about 10 μm to about 15 μm, or from about 15 μm to about 20 μm.

In at least some examples, the silicon-on-insulator substrate 506 can include a bulk layer. The bulk layer can be comprised of a silicon-containing material. In one or more examples, the bulk layer can be comprised primarily of silicon. For example, the bulk layer can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon. The bulk layer can also be referred to herein as a handle layer. In various examples, the thickness of the bulk layer can correspond to a reduced thickness in relation to an original thickness of the bulk layer. In one or more illustrative examples, the bulk layer can have a thickness from about 50 μm to about 800 μm, from about 100 μm to about 700 μm, from about 200 μm to about 600 μm, from about 100 μm to about 300 μm, from about 300 μm to about 500 μm, or from about 500 μm to about 700 μm.

Further, the semiconductor device 500 can include one or more passivation layers 508 disposed on the silicon-on-insulator substrate 506. The one or more passivation layers 508 can be comprised of one or more oxide materials and/or one or more nitride materials. For example, the one or more passivation layers 508 can be comprised of at least one of silicon oxide or silicon nitride. In one or more additional examples, the one or more passivation layers 508 can be comprised of polycrystalline silicon. In various examples, the one or more passivation layers 508 can be comprised of polycrystalline silicon doped with at least one of arsenic, phosphorus, or boron. The one or more passivation layers 508 can also be comprised of undoped polycrystalline silicon. The one or more passivation layers 508 can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

One or more coatings 510 can be disposed over outer surfaces of the semiconductor layer 502, the functional layer 504, the silicon-on-insulator substrate 506, and the one or more passivation layers 508. The one or more coatings 510 can include one or more resistive materials. To illustrate, the one or more coatings can include one or more oxide materials or one or more nitride materials. For example, the one or more coatings 510 can comprise at least one of silicon oxide, silicon nitride, titanium oxide, hafnium oxide, or aluminum oxide. The one or more coatings 510 can have a thickness from about 0.01 μm to about 3 μm, from about 0.05 μm to about 2 μm, from about 0.1 μm to about 1.5 μm, from about 0.01 μm to about 0.1 μm, from about 0.01 μm to about 0.05 μm, from about 0.1 μm to about 1 μm, from about 0.05 μm to about 0.5 μm, or from about 1 μm to about 2 μm.

A number of cavities can be formed by the layers and substrates of the semiconductor device 500. In the illustrative example of FIG. 5, the semiconductor device 500 can include a first cavity 512, a second cavity 514, and a third cavity 516. Side boundaries of the cavities 512, 514, 516 can be formed by locations of the semiconductor layer 502, the functional layer 504, the silicon-on-insulator substrate 506, the one or more passivation layers 508, and the one or more coatings 510. Although the illustrative example of FIG. 5 shows three cavities of the semiconductor device 500, the semiconductor device 500 can include a greater number of cavities. To illustrate, the semiconductor device 500 can include at least 10 cavities, at least 50 cavities, at least 100 cavities, at least 500 cavities, at least 1000 cavities, at least 2500 cavities, at least 5000 cavities, at least 10,000 cavities, or more. In various examples, the cavities 512, 514, 516 can form channels within the semiconductor device 500 in which one or more liquids can be disposed. The cavities 512, 514, 516 can have a width 518. The width 518 of the cavities 512, 514, 516 can be from 25 μm to about 400 μm, from about 50 μm to about 250 μm, from about 100 μm to about 200 μm, from about 25 μm to about 125 μm, from about 50 μm to about 150 μm, from about 50 μm to about 100 μm, or from about 100 μm to about 150 μm. In one or more illustrative examples, the cavities 512, 514, 516 can have a cylindrical shape. In these scenarios, the width 518 of the cavities 512, 514, 516 can correspond to a diameter.

The semiconductor device 500 can include a number of pores that are formed by openings in the one or more passivation layers 508 and the one or more coatings 510. In the illustrative example of FIG. 5, the semiconductor device 500 can include a first pore 520, a second pore 522, and third pore 524. Although the illustrative example of FIG. 5 shows three pores of the semiconductor device 500, the semiconductor device 500 can include a greater number of pores. For example, the semiconductor device 500 can include at least 100 pores, at least 500 pores, at least 1000 pores, at least 5000 pores, at least 10,000 pores, at least 25,000 pores, at least 50,000 pores, at least 100,000 pores, or more. The pores 520, 522, 524 can have a width 526 from about 0.1 μm to about 25 μm, from about 1 μm to about 20 μm, from about 5 μm to about 15 μm, from about 1 μm to about 10 μm, from about 10 μm to about 20 μm, from about 1 μm to about 5 μm, from about 0.1 μm to about 1 μm, from about 0.5 μm to about 2 μm, or from about 5 μm to about 10 μm.

Further, a spacing between the pores 520, 522, 524 can comprise a pitch of the semiconductor device 500. In one or more examples, the pitch of the semiconductor device 500 can be measured from a center of the first pore 520 to a center of the second pore 522. The pitch of the semiconductor device 500 can also be measured from a center of the second pore 522 to a center of the third pore 524. In at least some examples, the distance from a center of the first pore 520 to a center of the second pore 522 can be at least substantially the same as the distance from a center of the second pore 522 to a center of the third pore 524. In one or more illustrative examples, a pitch of the semiconductor device 500 can be from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

The semiconductor device 500 can also include a number of sites. The number of sites can be structured to apply electrical stimulation to a discrete amount of liquid located in individual sites. In the illustrative example of FIG. 5, the semiconductor device 500 includes a first site 528, a second site 530, and a third site 532. Although the illustrative example of FIG. 5 shows three sites of the semiconductor device 500, the semiconductor device 500 can include a greater number of sites. For example, the semiconductor device 500 can include at least 100 sites, at least 500 sites, at least 1000 sites, at least 5000 sites, at least 10,000 sites, at least 25,000 sites, at least 50,000 sites, at least 100,000 sites, or more.

The sites 528, 530, 532 can be defined by walls. The first site 528 can be enclosed by at least a first wall 534 and a second wall 536. In addition, the second site 530 can be enclosed by at least the second wall 536 and a third wall 538. Further, the third site 532 can be enclosed by the third wall 538 and a fourth wall 540. Although not shown in the illustrative cross sectional view of FIG. 5, the sites 528, 530, 532 can also be defined by walls disposed in rows perpendicular to the walls 534, 536, 538, 540. In this way, the sites 528, 530, 532 can be defined by a rectangular shape having boundaries formed by four walls. The sites 528, 530, 532 can have an inner width 542 from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

In one or more examples, the walls 534, 536, 538, 540 can be comprised of one or more polymeric materials. In at least some examples, the walls 534, 536, 538, 540 can be comprised of one or more photoresist materials. Additionally, the walls 534, 536, 538, 540 can be comprised of one or more hydrophobic materials and/or one or more low surface energy materials. In one or more illustrative examples, the walls 534, 536, 538, 540 can be comprised of an SU-8 photoresist, polytetrafluoroethylene (PTFE), one or more polyimides, one or more epoxy-based polymers, benzocyclobutene c (BCB), polydimethylsiloxane (PDMS), polymethylmethacrylate (PMMA), or one or more silanes. The one or more silanes can include octadecyltrimethoxysilane. In one or more illustrative examples, the walls 534, 536, 538, 540 can have a height 544 from about 1 μm to about 200 μm, from about 5 μm to about 100 μm, from about 10 μm to about 50 μm, from about 50 μm to about 100 μm, from about 1 μm to about 10 μm, from about 10 μm to about 25 μm, from about 50 μm to about 150 μm, or from about 100 μm to about 200 μm. In one or more additional illustrative examples, the walls 534, 536, 538, 540 can have a thickness 546 from about 0.1 μm to about 50 μm, from about 1 μm to about 30 μm, from about 5 μm to about 20 μm, from about 0.1 μm to about 1 μm, from about 0.5 μm to about 2 μm, from about 1 μm to about 5 μm, from about 1 μm to about 10 μm, from about 5 μm to about 15 μm, or from about 5 μm to about 10 μm.

The individual sites 528, 530, 532 can each include an electrode. The electrodes included in the individual sites 528, 530, 532 can be electrically connected to circuitry of the functional layer 504. In one or more examples, the electrodes can be electrically connected to circuitry of the functional layer 504 by vias formed in the one or more coatings 510, the one or more passivation layers 508, the silicon-on-insulator substrate 506, and, in at least some cases, at least a portion of the functional layer 504. In the illustrative example of FIG. 5, a first electrode 548 can be located in the first site 528, a second electrode 550 can be located in the second site 530, and a third electrode 552 can be located in the third site 532. In one or more examples, the first electrode 548, the second electrode 550, and the third electrode 552 can be used to apply at least one of voltage or current to liquid disposed in the respective sites 528, 530, 532. Additionally, at least one of the first electrode 548, the second electrode 550, and the third electrode 552 can be used to measure signals produced in response to electrical stimulation being provided to liquid disposed in the respective sites 528, 530, 532. The electrodes 548, 550, 552 can be comprised of one or more metallic materials. To illustrative, the electrodes 548, 550, 552 can be comprised of at least one of sliver chloride, platinum, alloys of platinum, gold, or alloys of gold.

In various examples, the semiconductor device 500 can also include at least one reference electrode 554. The at least one reference electrode 554 can be disposed in a cavity of the semiconductor device 500, such as the second cavity 514. In various additional examples, additional reference electrodes can be disposed in at least one of the first cavity 512 or the third cavity 516. The at least one reference electrode 554 can be comprised of at least one of sliver chloride, platinum, alloys of platinum, gold, or alloys of gold.

In one or more examples, liquid can be disposed in the sites 528, 530, 532. For example, the walls 534, 536, 538, 540 and the coated passivation layer(s) can comprise wells that can store a volume of liquid. For example, the walls 534, 536, 538, 540 can form side surfaces of the wells and the coated passivation layer(s) can comprise a floor of the wells. In one or more illustrative examples, volumes of liquid disposed in the individual sites 528, 530, 532 can include from about 1 picoliter (pL) to about 100 nanoliters (nL), from about 5 pL to about 10 nL, from about 10 pL to about 1 nL from about 1 pL to about 100 pL, from about 1 pL to about 50 pL, from about 1 pL to about 10 pL, from about 10 pL to about 500 pL, from about 10 pL to about 100 pL, from about 10 pL to about 50 pL, from about 50 pL to about 500 pL, from about 50 pL to about 250 pL, from about 100 pL to about 1 nL, from about 100 pL to about 500 pL, from about 500 pL to about 5 nL, from about 500 pL to about 2 nL, from about 500 pL to about 1 nL, from about 1 nL to about 100 nL, from about 1 nL to about 50 nL, or from about 1 nL to about 10 nL.

In the illustrative example of FIG. 5, an amount of liquid 556 can be dispensed into the second site 530. Although the illustrative example of FIG. 5 shows the amount of liquid 556 being dispensed into the second site 530, additional amounts of the liquid can also be dispensed into the first site 528, the third site 532, and other sites of the semiconductor device 500. In at least some examples, the amount of liquid 556 can comprise an aqueous solution having one or more biological cells, such as the example biological cell 558. In various examples, the amount of liquid 556 can form a droplet in the second site 530. For example, in scenarios where the walls 536, 538 are comprised of hydrophobic materials and the coating 510 is comprised of a hydrophilic material, the amount of liquid 556 can bead into droplets on the hydrophilic portions of the site and move away from the walls 536, 538. In this way, the droplet of the amount of liquid 556 in the second site can be electrically and fluidically isolated from droplets of liquid disposed in other sites of the semiconductor device 500. Accordingly, independent electrophysiological measurements can be produced for individual sites of the semiconductor device 500.

In one or more illustrative examples, first fluid can be disposed external to the cavities 512, 514, 516 and within the sites 528, 530, 532 and second fluid can be disposed within the cavities 512, 514, 516. The first fluid disposed external to the cavities 512, 514, 516 and within the sites 528, 530, 532 can correspond to the amount of liquid 556 and can include biological cells, one or more therapeutic compounds, one or more additional reagents, one or more combinations thereof, and the like. In at least some examples, the amount of liquid 556 can include at least one of NaCl, KCl, HEPES buffer, MgCl₂, CaCl₂, glucose, or ligands. In one or more additional examples, the second fluid disposed within the cavities 512, 514, 516 can include reagents that are used in relation to patch clamp processes and can include, in at least some examples, one or more therapeutic compounds.

In various examples, obtaining patch clamp measurements can include applying a pressure difference between the first fluid disposed in the sites 528, 530, 532 and the second fluid disposed in the cavities 512, 514, 516 to move biological cells 558 toward the respective pores 520, 522, 524. In one or more examples, the biological cells 558 can form a seal with the pores 520, 522, 524 such that the biological cells 558 are disposed at least partially within the pores 520, 522, 524 and on the respective floors of the sites 528, 530, 532 comprised of the coated passivation layer(s) 508. In at least some examples, a first pressure difference can be applied to move the biological cells 558 to form seals with the pores 520, 522, 524 and a second pressure difference can be applied to cause rupturing of the membranes of the biological cells 558. In one or more illustrative examples, the pressure difference can correspond to a negative pressure. In still other examples, a single pressure difference can be applied to move the biological cells 558 toward the respective pores 520, 522, 524 and to disrupt the membranes of the biological cells 558.

While the biological cells 558 are pressed against the floor of the respective sites 528, 530, 532 using the second amount of pressure to disrupt the membranes of the biological cells 558, a potential difference can be applied between the electrodes 548, 550, 552 and the reference electrode 554. In these scenarios, voltage clamp measurements can be collected. In one or more additional examples, while the biological cells 558 are pressed against the floor of the respective sites 528, 530, 532 using the second amount of pressure to disrupt the membranes of the biological cells 558, the electrodes 548, 550, 552 can deliver an amount of current to the biological cells 558 through the disrupted membranes of the biological cells 558 to collect current clamp measurements. The measurements collected during the voltage clamp and/or current clamp operations can be used to determine activity of biological cells 558 with respect to one or more therapeutic compounds and/or to one or more experimental conditions. In various examples, the amount of activity of the biological cells 558 during patch clamp operations can indicate a responsiveness of the biological cells 558 to at least one of the one or more therapeutic compounds or the one or more experimental conditions.

In one or more additional examples, the cavities 512, 514, 516 may not extend into the functional layer 504 and the semiconductor layer 502. In these scenarios, the semiconductor layer 502 and the functional layer 504 can represent a planar semiconductor substrate on which the cavities 512, 514, 516 are formed. In one or more further examples, the semiconductor device 500 can include one or more exhaust channels disposed above and/or within the sites 528, 530, 532 to alleviate pressure buildup, such as during manufacturing of the semiconductor device 500. In these instances, the exhaust channels can cause specified pressures to be present in one or more portions of the semiconductor device 500. In one or more illustrative examples, the exhaust channels can be configured to maintain pressures of about 101 kilopascals (kPa) in one or more portions of the semiconductor device 500.

In still other examples, the circuitry of the functional layer 504 can be located on a different part of a semiconductor chip on which the semiconductor device 500 is disposed. In these implementations, the semiconductor layer 502 can be replaced by a relatively inert substrate. For example, the semiconductor layer 502 can be replaced by a substrate formed from one or more polymeric materials or one or more glass materials. In one or more illustrative examples, the semiconductor layer 502 can be replaced by a polyimide substrate. Additionally, the functional layer 504 can be replaced by thin film transistors. To illustrate, individual electrodes 548, 550, 552 can be electrically coupled to thin film transistor switches. The switches can be in an on state or in an off state to control when patch clamp operations are to be performed in relation to liquids disposed in the sites 528, 530, 532. The thin film transistors can be electrically connected to a bus that is coupled to the functional circuitry disposed on the semiconductor chip.

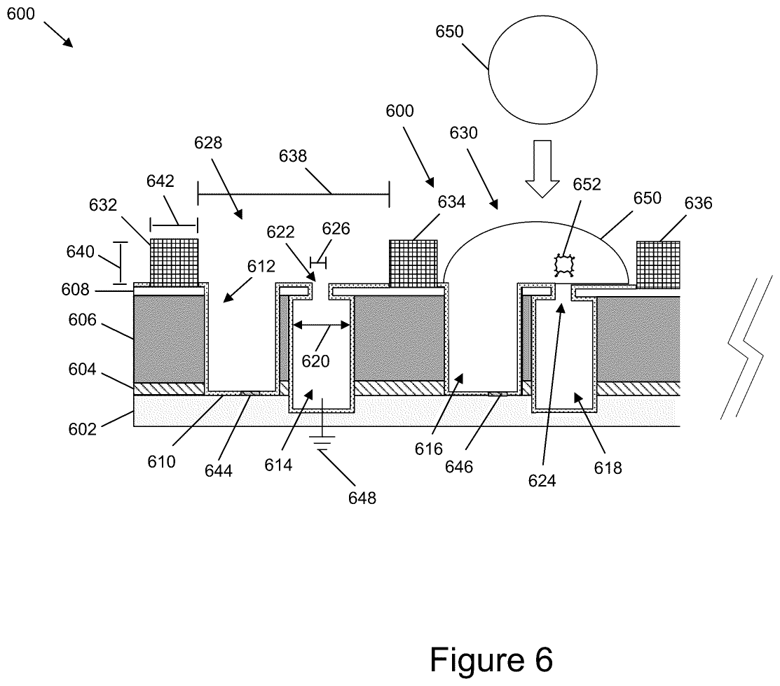

FIG. 6 is a diagram depicting a cross-section of at least a portion of a modified version of the second example semiconductor device 600 in which at least one of current or voltage can be applied to substances disposed in fluid, in accordance with one or more example implementations. In at least some examples, the modified version of the second example semiconductor device 600 can correspond to an implementation of the semiconductor device 102 described in relation to FIG. 1.

The semiconductor device 600 can include a semiconductor layer 602. The semiconductor layer 602 can include a silicon-containing layer. In one or more examples, the semiconductor layer 602 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

In addition, the semiconductor device 600 can include a functional layer 604 disposed on the semiconductor layer 602. The functional layer 604 can include circuitry formed on one or more substrates. In one or more examples, the functional layer 604 can include circuitry to apply at least one of voltage or current to liquid disposed on the semiconductor device 600 and to measure signals produced in response to the electrical stimulation provided to the liquid. In one or more illustrative examples, the functional layer 604 can include circuitry to perform at least one of voltage clamp operations, current clamp operations, or dynamic clamp operations. In various examples, the functional layer 604 can include analog-to-digital converters (ADCs), digital-to-analog converters (DACs), amplifiers, digital signal processing circuitry, one or more combinations thereof, and the like. The functional layer 604 can also include routing circuitry to carry electrical signals to locations on a die and/or on packaging of the semiconductor device 600 for further processing. The circuitry of the functional layer 604 can be produced using CMOS technologies.

The semiconductor device 600 can also include a silicon-on-insulator substrate 606. In one or more illustrative examples, the silicon-on-insulator substrate 606 can correspond to a micro-electro-mechanical systems (MEMS) substrate. The silicon-on-insulator substrate 606 can include one or more sublayers. For example, the silicon-on insulator substrate 606 can include an integrated circuit layer. The integrated circuit layer can include a silicon-containing layer. In at least some examples, circuitry can be disposed on the integrated circuit layer. In one or more examples, the integrated circuit layer can also be referred to herein as a device layer. The integrated circuit layer can have a thickness from about 10 μm to about 500 μm, from about 20 μm to about 400 μm, from about 30 μm to about 300 μm, from about 40 μm to about 200 μm, from about 20 μm to about 100 μm, from about 100 μm to about 200 μm, from about 50 μm to about 150 μm, from about 150 μm to about 250 μm, from about 200 μm to about 300 μm, from about 250 μm to about 350 μm, or from about 300 μm to about 400 μm,

Additionally, the silicon-on-insulator substrate 606 can include an insulator layer disposed under the integrated circuit layer. In one or more examples, the insulator layer can be comprised of an oxide-containing material. For example, the insulator layer can be comprised of silicon dioxide. In one or more additional examples, the insulator layer can be comprised of a sapphire-containing materials. In various examples, the insulator layer can be referred to herein as a buried oxide layer. The insulator layer can have a thickness from about 0.1 μm to about 25 μm, from about 0.5 μm to about 20 μm, from about 1 μm to about 15 μm, from about 2 μm to about 10 μm, from about 1 μm to about 5 μm, from about 5 μm to about 10 μm, from about 10 μm to about 15 μm, or from about 15 μm to about 20 μm.

In at least some examples, the silicon-on-insulator substrate 606 can include a bulk layer. The bulk layer can be comprised of a silicon-containing material. In one or more examples, the bulk layer can be comprised primarily of silicon. For example, the bulk layer can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon. The bulk layer can also be referred to herein as a handle layer. In various examples, the thickness of the bulk layer can correspond to a reduced thickness in relation to an original thickness of the bulk layer. In one or more illustrative examples, the bulk layer can have a thickness from about 50 μm to about 800 μm, from about 100 μm to about 700 μm, from about 200 μm to about 600 μm, from about 100 μm to about 300 μm, from about 300 μm to about 500 μm, or from about 500 μm to about 700 μm.

Further, the semiconductor device 600 can include one or more passivation layers 608 disposed on the silicon-on-insulator substrate 606. The one or more passivation layers 608 can be comprised of one or more oxide materials and/or one or more nitride materials. For example, the one or more passivation layers 608 can be comprised of at least one of silicon oxide or silicon nitride. In one or more additional examples, the one or more passivation layers 608 can be comprised of polycrystalline silicon. In various examples, the one or more passivation layers 608 can be comprised of polycrystalline silicon doped with at least one of arsenic, phosphorus, or boron. The one or more passivation layers 608 can also be comprised of undoped polycrystalline silicon. The one or more passivation layers 608 can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

One or more coatings 610 can be disposed over outer surfaces of the semiconductor layer 602, the functional layer 604, the silicon-on-insulator substrate 606, and the one or more passivation layers 608. The one or more coatings 610 can include one or more resistive materials. To illustrate, the one or more coatings can include one or more oxide materials or one or more nitride materials. For example, the one or more coatings 610 can comprise at least one of silicon oxide, silicon nitride, titanium oxide, hafnium oxide, or aluminum oxide. The one or more coatings 610 can have a thickness from about 0.01 μm to about 3 μm, from about 0.05 μm to about 2 μm, from about 0.1 μm to about 1.5 μm, from about 0.01 μm to about 0.1 μm, from about 0.01 μm to about 0.05 μm, from about 0.1 μm to about 1 μm, from about 0.05 μm to about 0.5 μm, or from about 1 μm to about 2 μm.

A number of cavities can be formed by the layers and substrates of the semiconductor device 600. In the illustrative example of FIG. 6, the semiconductor device 600 can include a first cavity 612, a second cavity 614, a third cavity 616, and a fourth cavity 618. Side boundaries of the first cavity 612 and the third cavity 616 can be formed by locations of the functional layer 604, the silicon-on-insulator substrate 606, the one or more passivation layers 608, and the one or more coatings 610. Additionally, the side boundaries of the second cavity 614 and the fourth cavity 618 can be formed by locations of the semiconductor layer 602, the functional layer 604, the silicon-on-insulator substrate 606, the one or more passivation layers 608, and the one or more coatings 610. Although the illustrative example of FIG. 6 shows four cavities of the semiconductor device 600, the semiconductor device 600 can include a greater number of cavities. To illustrate, the semiconductor device 600 can include at least 10 cavities, at least 50 cavities, at least 100 cavities, at least 500 cavities, at least 1000 cavities, at least 2500 cavities, at least 5000 cavities, at least 10,000 cavities, at least 20,000 cavities, or more. In various examples, the cavities 612, 614, 616, 618 can form channels within the semiconductor device 600 in which one or more liquids can be disposed. The cavities 612, 614, 616, 618 can have a width 620. The width 620 of the cavities 612, 614, 616, 618 can be from 25 μm to about 400 μm, from about 50 μm to about 250 μm, from about 100 μm to about 200 μm, from about 25 μm to about 125 μm, from about 50 μm to about 150 μm, from about 50 μm to about 100 μm, or from about 100 μm to about 150 μm. In one or more illustrative examples, the cavities 612, 614, 616, 618 can have a cylindrical shape. In these scenarios, the width 620 of the cavities 612, 614, 616, 618 can correspond to a diameter.

The semiconductor device 600 can include a number of pores that are formed by openings in the one or more passivation layers 608 and the one or more coatings 610. In the illustrative example of FIG. 6, the semiconductor device 600 can include a first pore 622 and a second pore 624. Although the illustrative example of FIG. 6 shows two pores of the semiconductor device 600, the semiconductor device 600 can include a greater number of pores. For example, the semiconductor device 600 can include at least 100 pores, at least 500 pores, at least 1000 pores, at least 5000 pores, at least 10,000 pores, at least 25,000 pores, at least 50,000 pores, at least 100,000 pores, or more. The pores 622, 624 can have a width 626 from about 0.1 μm to about 25 μm, from about 1 μm to about 20 μm, from about 5 μm to about 15 μm, from about 1 μm to about 10 μm, from about 10 μm to about 20 μm, from about 1 μm to about 5 μm, from about 0.1 μm to about 1 μm, from about 0.5 μm to about 2 μm, or from about 5 μm to about 10 μm.

Further, a spacing between the pores 622, 624 can comprise a pitch of the semiconductor device 600. In one or more examples, the pitch of the semiconductor device 600 can be measured from a center of the first pore 622 to a center of the second pore 624. In one or more illustrative examples, a pitch of the semiconductor device 600 can be from about 50 μm to about 1000 μm, from about 100 μm to about 800 μm, from about 200 μm to about 600 μm, from about 100 μm to about 500 μm, from about 200 μm to about 600 μm, from about 300 μm to about 700 μm, from about 100 μm to about 300 μm, from about 200 μm to about 400 μm, from about 300 μm to about 500 μm, from about 400 μm to about 600 μm, or from about 500 μm to about 700 μm.

The semiconductor device 600 can also include a number of sites. The number of sites can be structured to apply electrical stimulation to a discrete amount of liquid located in individual sites. In the illustrative example of FIG. 6, the semiconductor device 600 includes a first site 628 and a second site 630. Although the illustrative example of FIG. 6 shows two sites of the semiconductor device 600, the semiconductor device 600 can include a greater number of sites. For example, the semiconductor device 600 can include at least 100 sites, at least 500 sites, at least 1000 sites, at least 5000 sites, at least 10,000 sites, at least 25,000 sites, at least 50,000 sites, at least 100,000 sites, or more.

The sites 628, 630 can be defined by walls. The first site 628 can be enclosed by at least a first wall 632 and a second wall 634. In addition, the second site 630 can be enclosed by at least the second wall 634 and a third wall 636. Although not shown in the illustrative cross sectional view of FIG. 6, the sites 628, 630 can also be defined by walls disposed in rows perpendicular to the walls 632, 634, 636. In this way, the sites 628, 630 can be defined by a rectangular shape having boundaries formed by four walls. The sites 628, 630 can have an inner width 638 from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

In one or more examples, the walls 632, 634, 636 can be comprised of one or more polymeric materials. In at least some examples, the walls 632, 634, 636 can be comprised of one or more photoresist materials. Additionally, the walls 632, 634, 636 can be comprised of one or more hydrophobic materials and/or one or more low surface energy materials. In one or more illustrative examples, the walls 632, 634, 636 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In one or more illustrative examples, the walls 632, 634, 636 can have a height 640 from about 1 μm to about 200 μm, from about 5 μm to about 100 μm, from about 10 μm to about 50 μm, from about 50 μm to about 100 μm, from about 1 μm to about 10 μm, from about 10 μm to about 25 μm, from about 50 μm to about 150 μm, or from about 100 μm to about 200 μm. In one or more additional illustrative examples, the walls 632, 634, 636 can have a thickness 642 from about 0.1 μm to about 50 μm, from about 1 μm to about 30 μm, from about 5 μm to about 20 μm, from about 0.1 μm to about 1 μm, from about 0.5 μm to about 2 μm, from about 1 μm to about 5 μm, from about 1 μm to about 10 μm, from about 5 μm to about 15 μm, or from about 5 μm to about 10 μm.

The individual sites 628, 630 can each include an electrode. The electrodes included in the individual sites 628, 630 can be electrically connected to circuitry of the functional layer 604. In one or more examples, the electrodes can be disposed on the functional layer 604. In the illustrative example of FIG. 6, a first electrode 644 can be located in the first site 628 and a second electrode 646 can be located in the second site 630. In one or more examples, the first electrode 644 and the second electrode 646 can be used to apply at least one of voltage or 10 current to liquid disposed in the respective sites 628, 630. Additionally, at least one of the first electrode 644 and the second electrode 646 can be used to measure signals produced in response to electrical stimulation being provided to liquid disposed in the respective sites 628, 630. The electrodes 644, 646 can be comprised of one or more metallic materials. To illustrative, the electrodes 644, 646 can be comprised of at least one of sliver chloride, platinum, alloys of platinum, gold, or alloys of gold.

In various examples, the semiconductor device 600 can also include at least one reference electrode 648. The at least one reference electrode 648 can be disposed in a cavity of the semiconductor device 600, such as the second cavity 614. In various additional examples, additional reference electrodes can be disposed in at least one of the first cavity 612, the third cavity 616, or the fourth cavity 618. The at least one reference electrode 646 can be comprised of at least one of sliver chloride, platinum, alloys of platinum, gold, or alloys of gold.

In one or more examples, liquid can be disposed in the sites 628, 630. For example, the walls 632, 634, 636 and the coated passivation layer(s) can comprise wells that can store a volume of liquid. For example, the walls 632, 634, 636 can form side surfaces of the wells and the coated passivation layer(s) can comprise a floor of the wells. In one or more illustrative examples, volumes of liquid disposed in the individual sites 628, 630 can include from about 1 picoliter (pL) to about 100 nanoliters (nL), from about 5 pL to about 10 nL, from about 10 pL to about 1 nL from about 1 pL to about 100 pL, from about 1 pL to about 50 pL, from about 1 pL to about 10 pL, from about 10 pL to about 500 pL, from about 10 pL to about 100 pL, from about 10 pL to about 50 pL, from about 50 pL to about 500 pL, from about 50 pL to about 250 pL, from about 100 pL to about 1 nL, from about 100 pL to about 500 pL, from about 500 pL to about 5 nL, from about 500 pL to about 2 nL, from about 500 pL to about 1 nL, from about 1 nL to about 100 nL, from about 1 nL to about 50 nL, or from about 1 nL to about 10 nL.

In the illustrative example of FIG. 6, an amount of liquid 650 can be dispensed into the second site 630. Although the illustrative example of FIG. 6 shows the amount of liquid 650 being dispensed into the second site 630, additional amounts of the liquid can also be dispensed into the first site 628 and other sites of the semiconductor device 600. In at least some examples, the amount of liquid 650 can comprise an aqueous solution having one or more biological cells, such as the example biological cell 652. In various examples, the amount of liquid 650 can form a droplet in the second site 630. For example, in scenarios where the walls 634, 636 are comprised of hydrophobic materials and the coating 610 is comprised of a hydrophilic material, the amount of liquid 650 can bead into droplets on the hydrophilic portions of the site and move away from the walls 634, 636. In this way, the droplet of the amount of liquid 650 in the second site 630 can be electrically and fluidically isolated from droplets of liquid disposed in other sites of the semiconductor device 600. Accordingly, independent electrophysiological measurements can be produced for individual sites of the semiconductor device 600.

In one or more illustrative examples, first fluid can be disposed within the first cavity 612 and the third cavity 616 within the sites 628, 630 and second fluid can be disposed within the second cavity 614 and the fourth cavity 618. The first fluid disposed in the first cavity 612 and the third cavity 616 and within the sites 628, 630 can correspond to the amount of liquid 650 and can include biological cells, one or more therapeutic compounds, one or more additional reagents, one or more combinations thereof, and the like. In one or more additional examples, the second fluid disposed within the second cavity 614 and the fourth cavity 618 can include reagents that are used in relation to patch clamp processes and can include, in at least some examples, one or more therapeutic compounds.

In various examples, obtaining patch clamp measurements can include applying a pressure difference between the first fluid disposed in the first cavity 612, the second cavity 614, and the sites 628, 630 and the second fluid disposed in the second cavity 614 and the fourth cavity 618 to move biological cells 652 toward the respective pores 622, 624. In one or more examples, the biological cells 652 can form a seal with the pores 622, 624 such that the biological cells 652 are disposed at least partially within the pores 622, 624 and on the respective surfaces of the coated passivation layers around the pores 622, 624. In at least some examples, a first pressure difference can be applied to move the biological cells 652 to form seals with the pores 622, 624 and a second pressure difference can be applied to cause rupturing of the membranes of the biological cells 652. In one or more illustrative examples, the pressure difference can correspond to a negative pressure. In still other examples, a single pressure difference can be applied to move the biological cells 652 toward the respective pores 622, 624 and to disrupt the membranes of the biological cells 652.

While the biological cells 652 are pressed against the surface of the respective sites 628, 630 around the pores 622, 624 using the second amount of pressure to disrupt the membranes of the biological cells 652, a potential difference can be applied between the electrodes 644, 646 and the reference electrode 648. In these scenarios, voltage clamp measurements can be collected. In one or more additional examples, while the biological cells 652 are pressed against the surface of the respective sites 628, 630 around the pores 622, 624 using the second amount of pressure to disrupt the membranes of the biological cells 652, the electrodes 644, 646 can deliver an amount of current to the biological cells 652 through the disrupted membranes of the biological cells 652 to collect current clamp measurements. The measurements collected during the voltage clamp and/or current clamp operations can be used to determine activity of biological cells 652 with respect to one or more therapeutic compounds and/or to one or more experimental conditions. In various examples, the amount of activity of the biological cells 652 during patch clamp operations can indicate a responsiveness of the biological cells 652 to at least one of the one or more therapeutic compounds or the one or more experimental conditions.

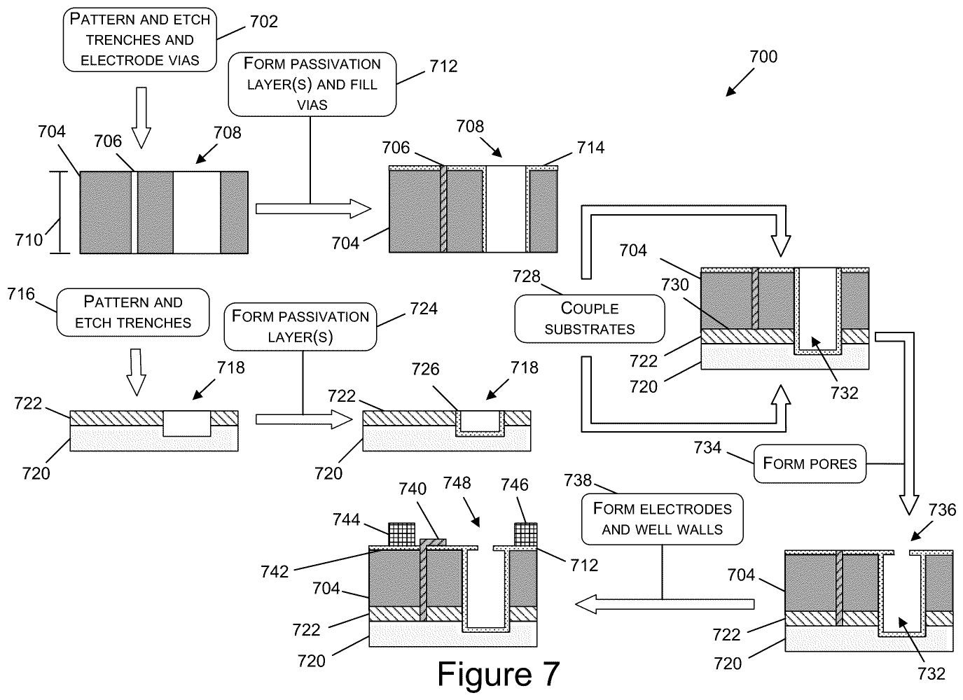

FIG. 7 is a diagram depicting a process 700 of manufacturing a second example semiconductor device in which at least one of current or voltage can be applied to substances disposed in fluid, in accordance with one or more example implementations. In one or more illustrative examples, the process 700 can be implemented to produce semiconductor devices having one or more features of the semiconductor device 500 described in relation to FIG. 5.

The process 700 can include, at 702, patterning and etching trenches and electrode vias in a silicon-on-insulator substrate 704. For example, a through-silicon via 706 can be formed in the silicon-on-insulator substrate 704 and a trench 708 can be formed in the silicon-on-insulator substrate 704. In one or more examples, the through-silicon via 706 can correspond to a location of a portion of an electrode of a semiconductor device and the trench 708 can correspond to a location of a cavity of the semiconductor device. The through-silicon via 706 and the trench 708 can be formed by one or more lithographic processes. The one or more lithographic processes can include depositing a photoresist layer or film on the silicon-on-insulator substrate 704 according to a pattern and then exposing the photoresist to electromagnetic radiation. In scenarios where the photoresist is a positive photoresist, the location of the through-silicon via 706 and the location of the trench 708 are exposed to electromagnetic radiation and the exposed portions of the positive photoresist are removed by the developer solution. In situations where the photoresist is a negative photoresist, the location of the through-silicon via 706 and the location of the trench 708 are not exposed to electromagnetic radiation and the unexposed portions of the negative photoresist are removed by the developer solution. In this way, the photoresist pattern forms an etch mask for subsequent etching processes where the through-holes will be formed. In one or more illustrative examples, the through-silicon via 706 and the trench 708 can be formed using a deep reactive-ion etching process. In one or more additional illustrative examples, the through-silicon via 706 and the trench 708 can be formed using a potassium hydroxide etch process or a tetramethylammonium hydroxide etching process.

The silicon-on-insulator substrate 704 can include a device layer, an insulator layer disposed under the integrated circuit layer, and a bulk layer disposed under the insulator layer. The insulator layer can be comprised of one or more oxide materials. In addition, the bulk layer can be comprised of a silicon-containing material. In one or more examples, the bulk layer can be comprised primarily of silicon. The silicon-on-insulator substrate 704 can have a thickness 710 from about 50 μm to about 1000 μm, from about 100 μm to about 800 μm, from about 200 μm to about 600 μm, from about 100 μm to about 500 μm, from about 500 μm to about 1000 μm, from about 50 μm to about 200 μm, from about 100 μm to about 300 μm, from about 200 μm to about 400 μm, from about 300 μm to about 500 μm, from about 400 μm to about 600 μm, from about 500 μm to about 700 μm, from about 600 μm to about 800 μm, from about 700 μm to about 900 μm, or from about 800 μm to about 1000 μm.

The process 700 can also include, at 712, forming one or more passivation layers 714 and filling the through-silicon vias 706 with a conductive material. For example, one or more passivation layers 714 can be formed on the side walls of the through-silicon via 706, the side walls of the trench 708, and on the silicon-on-insulator substrate 704. The one or more passivation layers 714 can be comprised of at least one of one or more resistive materials. For example, the one or more passivation layers 714 can be comprised of at least one of one or more oxides or one or more nitrides. To illustrate, the one or more passivation layers 714 can be comprised of at least one of silicon oxide or silicon nitride. In scenarios where the one or more passivation layers 714 include an oxide material, the one or more oxide-containing passivation layers can be formed by at least one of thermal oxidation of the silicon-on-insulator substrate 704, chemical vapor deposition, physical vapor deposition, plasma enhanced chemical vapor deposition, thermal evaporation, or electron beam evaporation. Additionally, in situations where the one or more passivation layers 714 include a nitride material, the one or more nitride-containing passivation layers can be formed by at least one of low pressure chemical vapor deposition, plasma enhanced chemical vapor deposition, thermal atomic layer deposition, or plasma enhanced atomic layer deposition.

The conductive materials used to fill the through-silicon-vias 706 can include at least one of copper, alloys of copper, tungsten, alloys of tungsten or a polycrystalline silicon material. In one or more illustrative examples where the conductive material is polycrystalline silicon, the polycrystalline silicon can be doped with at least one of boron, phosphorus, or arsenic. The conductive material can be formed in the through-silicon-vias 706 by one or more electroplating processes or one or more physical vapor deposition processes.

Additionally, the process 700 can include, at 716, patterning and etching trenches 718 in a semiconductor substrate. The semiconductor substrate can include a semiconductor layer 720. The semiconductor layer 720 can include a silicon-containing layer. In one or more examples, the semiconductor layer 720 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon. The semiconductor substrate can also include a functional layer 722 disposed on the semiconductor layer 720. The functional layer 722 can include circuitry formed on one or more substrates. In one or more examples, the functional layer 722 can include circuitry to apply at least one of voltage or current to liquid and to measure signals produced in response to the electrical stimulation provided to the liquid. In one or more illustrative examples, the functional layer 722 can include circuitry to perform at least one of voltage clamp operations, current clamp operations, or dynamic clamp operations. In one or more illustrative examples, the trenches 718 can be formed using one or more lithographic processes. In one or more additional illustrative examples, the trenches 718 can be formed by deep reactive ion etching.

The process 700 can include, at 724, forming one or more passivation layers 726 on the semiconductor substrate. For example, the one or more passivation layers 726 can be formed within the trenches 718. In one or more additional examples, the one or more passivation layers 726 can be formed on the functional layer 722. The one or more passivation layers 726 can be comprised of at least one of one or more resistive materials. For example, the one or more passivation layers 726 can be comprised of at least one of one or more oxides or one or more nitrides. To illustrate, the one or more passivation layers 726 can be comprised of at least one of silicon oxide or silicon nitride. The one or more passivation layers 726 can be formed by at least one of thermal oxidation of the semiconductor substrate, chemical vapor deposition, physical vapor deposition, plasma enhanced chemical vapor deposition, thermal evaporation, or electron beam evaporation.

At 728, the process 700 can include coupling the substrates. For example, the silicon-on-insulator substrate 704 formed by operations 702 and 712 can be coupled to the semiconductor substrate formed by operations 716 and 724. To illustrate, the functional layer 722 of the semiconductor substrate can be coupled to a surface 730 of the silicon-on-insulator substrate 704. In one or more illustrative examples, the substrates can be coupled using direct wafer bonding techniques. In one or more additional illustrative examples, the substrate can be coupled using mediated wafer bonding techniques. In various examples, the coupling of the substrates can include aligning the trenches 708 formed in the silicon-on-insulator substrate 704 with the trenches 718 formed in the semiconductor substrate to form a combined trench 732. The coupling of the substrate at 728 can also include coupling the filled through-silicon vias 706 with circuitry disposed on the functional layer 722 of the semiconductor substrate. In addition, in at least some examples, a thickness of the silicon-on-insulator substrate 704 can be reduced by performing at least one of wet etching, dry etching, wafer back grinding, or chemical-mechanical polishing with respect to the handle layer of the silicon-on-insulator substrate 704.

Further, the process 700 can include, at 734, forming pores 736 over the combined trenches 732. In one or more examples, the pores 736 can be formed in one or more passivation layers formed over the trenches 732. In various examples, the one or more passivation layers can be formed over the trenches 708 prior to the substrates being coupled at 728. In one or more additional examples, the one or more passivation layers can be formed over the combined trenches 732 after coupling the substrates at 728. The pores 736 can be formed using one or more pattern and etching lithographic processes.

At 738, the process 700 can include forming electrodes 740 on one or more passivation layers disposed on an additional surface 742 of the silicon-on-insulator substrate 704. The additional surface 742 of the silicon-on-insulator substrate 704 can be disposed at least substantially parallel with respect to the surface 730 of the silicon-on-insulator substrate 704. The electrodes 740 can be formed by at least one of electroplating, sputter deposition, chemical vapor deposition, or physical vapor deposition. The electrodes 740 can be comprised of one or more metals that can be used to pass current through an aqueous liquid. In one or more examples, the electrodes 740 can be comprised of at least one of silver chloride, platinum, alloys of platinum, gold, or alloys of gold.

Operation 738 can also include forming walls on the one or more passivation layers disposed on the additional surface 742 of the silicon-on-insulator substrate 704, such as a first wall 744 and a second wall 746. In one or more examples, the first wall 744 and the second wall 746 can form a well 748 in which liquid can be disposed. The walls 744, 746 can separate the well 748 from other wells of the semiconductor device. In various examples, the walls 744, 746 can be formed from one or more hydrophobic materials. To illustrate, the walls 744, 746 can be formed from one or more photoresist materials. In one or more illustrative examples, the walls 744, 746 can be formed from at least one of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In at least some examples, the walls 744, 746 can be formed using one or more lithographic processes to pattern one or more photoresist materials according to the locations of the walls 744, 746.

Although the illustrative example of FIG. 7 describes the formation of features of a single site of a semiconductor device, the process 700 can be used to form features of multiple sites of a semiconductor device. For example, the process 700 can be used to form features of at least 5 sites, at least 20 sites, at least 50 sites, at least 100 sites, at least 200 sites, at least 500 sites, at least 1000 sites, at least 2000 sites, at least 5000 sites, at least 8000 sites, at least 10,000 sites, at least 25,000 sites, at least 50,000 sites, at least 75,000 sites, at least 100,000 sites, or more.

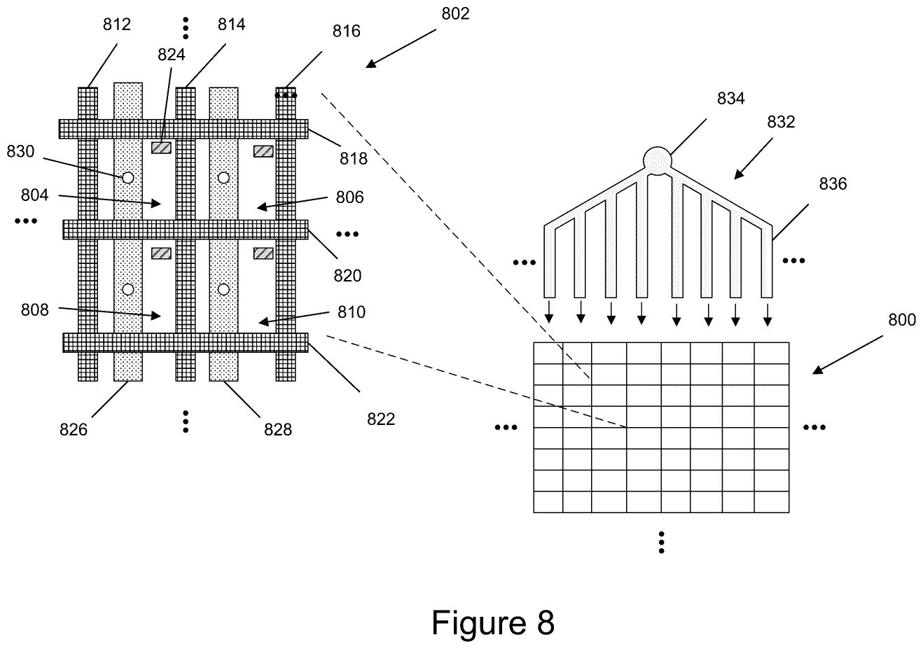

FIG. 8 is a diagram depicting a top view of a second example semiconductor device having an array of sites 800 in which at least one of current or voltage can be applied to substances disposed in fluid, in accordance with one or more example implementations. In one or more examples, the array of sites 800 can include sites that correspond to sites 528, 530, 532 described in relation to FIG. 5 and a site manufactured according to the process 700 described in relation to FIG. 7. In one or more examples, the array of sites 800 can have tens of sites, hundreds of sites, thousands of sites, up to tens of thousands of sites, or more.

The subsection includes a number of individual sites and a number of features of the individual sites. For example, the subsection 802 includes a first site 804, a second site 806, a third site 808, and a fourth site 810. The boundaries of the sites 804, 806, 808, 810 can be formed by rows and columns of walls comprised of one or more polymeric materials. For example, the boundaries of the sites 804, 806, 808, 810 can be formed by a first wall section 812 comprised of one or more polymeric materials, a second wall section 814 comprised of the one or more polymeric materials, and a third wall section 816 comprised of the one or more polymeric materials. Additionally, the boundaries of the sites 804, 806, 808, 810 can be formed by a fourth wall section 818 comprised of the one or more polymeric materials, a fifth wall section 820 comprised of the one or more polymeric materials, and a sixth wall section 822 comprised of the one or more polymeric materials. The first wall section 812, the second wall section 814, and the third wall section 816 can be disposed at least substantially perpendicular with respect to the fourth wall section 818, the fifth wall section 820, and the sixth wall section 822.

Individual sites 804, 806, 808, 810 can include an electrode 824. Additionally, a first membrane section 826 can be disposed in the sites 804, 808 and a second membrane section 828 can be disposed in the sites 806, 810. The membrane sections 826, 828 can be comprised of one or more hydrophilic materials. In one or more examples, the one or more hydrophilic materials can include one or more oxide-containing materials that comprise at least one passivation layer formed over one or more layers of a semiconductor substrate of the semiconductor device. Further, the individual sites 804, 806, 808 810 can include a pore 830 that is disposed in a respective membrane section 826, 828.

FIG. 8 also illustrates an example fluid distribution system 832. The fluid distribution system 832 includes a fluidic inlet port 834 that receives fluid from one or more fluid sources. The fluid distribution system 832 and the one or more fluid sources can correspond to a fluid delivery system. The fluid distribution system 832 can also include a number of channels 836. Individual channels 836 can be disposed throughout columns of sites of the array of sites 800 and dispense fluid to sites of the array of sites 800. In one or more examples, a portion of the channels 836 can correspond to the cavities 512, 514, 516 described in relation to FIG. 5.

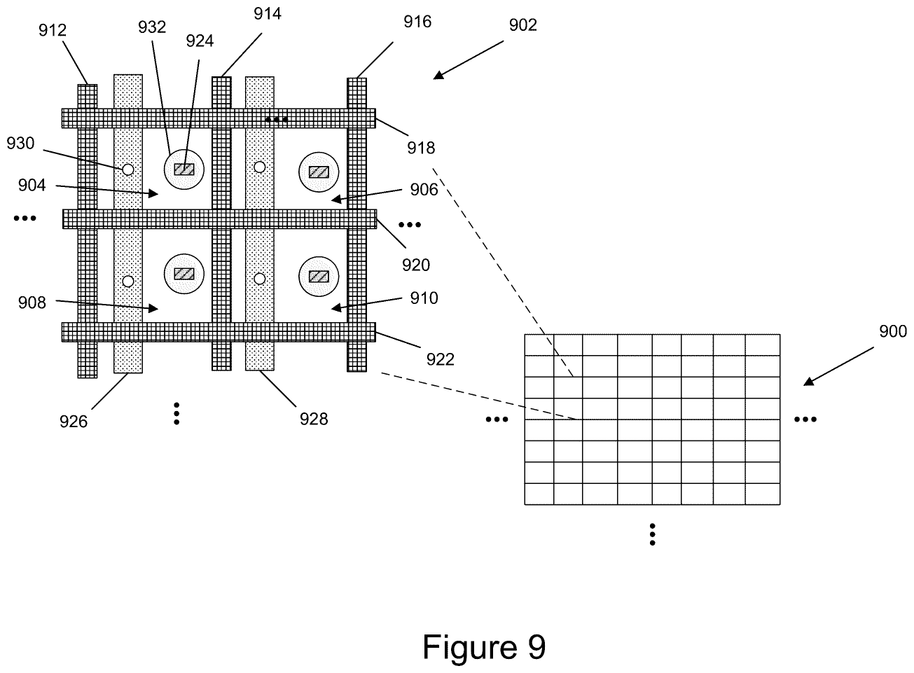

FIG. 9 is a diagram depicting a top view of a modified version of a second example semiconductor device having an array of sites 900 in which at least one of current or voltage can be applied to substances disposed in fluid, in accordance with one or more example implementations. In one or more examples, the array of sites 900 can include sites that correspond to sites 628, 630 described in relation to FIG. 6. In one or more examples, the array of sites 900 can have tens of sites, hundreds of sites, thousands of sites, up to tens of thousands of sites, or more.

A subsection 902 of the array of sites 900 includes a number of individual sites and a number of features of the individual sites. For example, the subsection 902 includes a first site 904, a second site 906, a third site 908, and a fourth site 910. The boundaries of the sites 904, 906, 908, 910 can be formed by rows and columns of walls comprised of one or more polymeric materials. For example, the boundaries of the sites 904, 906, 908, 910 can be formed by a first wall section 912 comprised of one or more polymeric materials, a second wall section 914 comprised of the one or more polymeric materials, and a third wall section 916 comprised of the one or more polymeric materials. Additionally, the boundaries of the sites 904, 906, 908, 910 can be formed by a fourth wall section 918 comprised of the one or more polymeric materials, a fifth wall section 920 comprised of the one or more polymeric materials, and a sixth wall section 922 comprised of the one or more polymeric materials. The first wall section 912, the second wall section 914, and the third wall section 916 can be disposed at least substantially perpendicular with respect to the fourth wall section 918, the fifth wall section 920, and the sixth wall section 922.

Individual sites 904, 906, 908, 910 can include an electrode 924. Additionally, a first membrane section 926 can be disposed in the sites 904, 908 and a second membrane section 928 can be disposed in the sites 906, 910. The membrane sections 926, 928 can be comprised of one or more hydrophilic materials. In one or more examples, the one or more hydrophilic materials can include one or more oxide-containing materials that comprise at least one passivation layer formed over one or more layers of a semiconductor substrate of the semiconductor device. Further, the individual sites 904, 906, 908 910 can include a pore 930 that is disposed in a respective membrane section 926, 928. In still other examples, the electrode 924 can be disposed in an electrode cavity 932 that is separate from the membrane section 926. In these scenarios, the electrode cavity 932 can correspond to the cavity 612 or 616 of FIG. 6 and the membrane section 926 in which the pore 930 is disposed can correspond to the cavity 614 or 618 of FIG. 6.

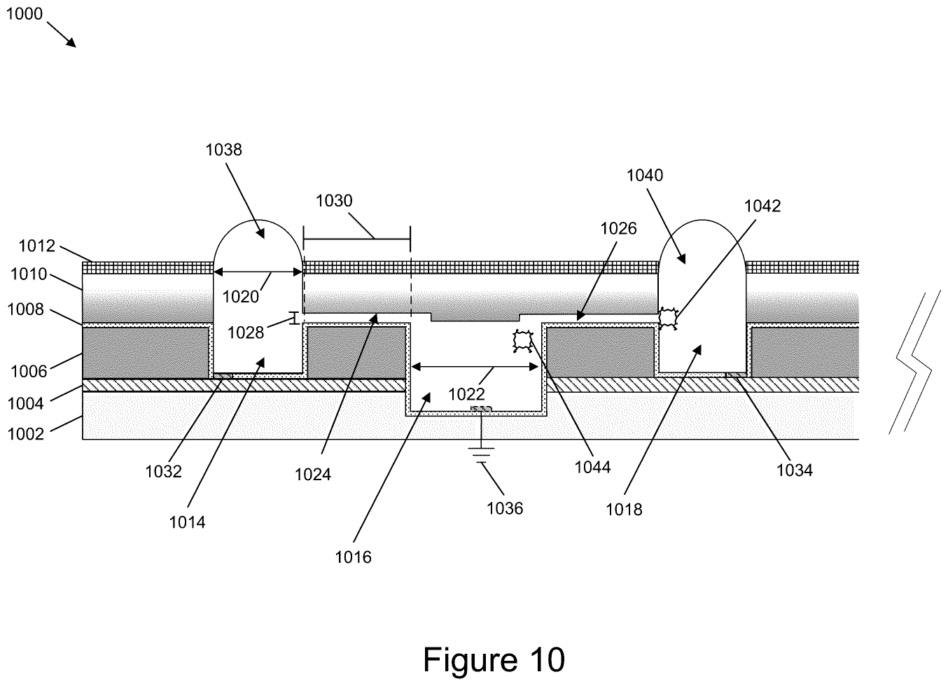

FIG. 10 is a diagram depicting a cross-section of at least a portion of a third example semiconductor device 1000 in which at least one of current or voltage can be applied to substances disposed in fluid, in accordance with one or more example implementations. In at least some examples, the third example semiconductor device 1000 can correspond to an implementation of the semiconductor device 102 described in relation to FIG. 1.

The semiconductor device 1000 can include a semiconductor layer 1002. The semiconductor layer 1002 can include a silicon-containing layer. In one or more examples, the semiconductor layer 1002 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

In addition, the semiconductor device 1000 can include a functional layer 1004 disposed on the semiconductor layer 1002. The functional layer 1004 can include circuitry formed on one or more substrates. In one or more examples, the functional layer 1004 can include circuitry to apply at least one of voltage or current to liquid disposed on the semiconductor device 1000 and to measure signals produced in response to the electrical stimulation provided to the liquid. In one or more illustrative examples, the functional layer 1004 can include circuitry to perform at least one of voltage clamp operations, current clamp operations, or dynamic clamp operations. In various examples, the functional layer 1004 can include analog-to-digital converters (ADCs), digital-to-analog converters (DACs), amplifiers, digital signal processing circuitry, one or more combinations thereof, and the like. The functional layer 1004 can also include routing circuitry to carry electrical signals to locations on a die and/or on packaging of the semiconductor device 1000 for further processing. The circuitry of the functional layer 1004 can be produced using CMOS technologies.

The semiconductor device 1000 can also include a polycrystalline silicon layer 1006. The polycrystalline silicon layer 1006 can be disposed on the functional layer 1004. In one or more examples, the polycrystalline silicon layer 1006 can be undoped. In one or more additional examples, the polycrystalline silicon layer 1006 can be doped. In scenarios where the polycrystalline silicon layer 1006 is doped, dopants of the polycrystalline silicon layer 1006 can include at least one of boron, arsenic, or phosphorus.

Further, the semiconductor device 1000 can include one or more passivation layers 1008 disposed on the polycrystalline silicon layer 1006, disposed on the functional layer 1004, or disposed on portions of the semiconductor layer 1002. The one or more passivation layers 1008 can be comprised of one or more oxide materials and/or one or more nitride materials. For example, the one or more passivation layers 1008 can be comprised of at least one of silicon oxide or silicon nitride. The one or more passivation layers 1008 can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm. In various examples, the one or more passivation layers 1008 can include one or more coatings comprised of at least one of silicon oxide or silicon nitride and having a thickness from about 0.001 μm to about 0.3 μm, from about 0.005 μm to about 0.2 μm, from about 0.01 μm to about 0.15 μm, from about 0.01 μm to about 0.1 μm, from about 0.01 μm to about 0.05 μm, from about 0.1 μm to about 1 μm, from about 0.05 μm to about 0.5 μm, or from about 1 μm to about 2 μm.

The semiconductor device 1000 can also include a substrate 1010 comprised of one or more glass materials. In addition, the semiconductor device 1000 can include a separator layer 1012. The separator layer 1012 can isolate portions of the semiconductor device 1000 in which one or more liquids are disposed. The separator layer 1012 can be comprised of one or more polymeric materials. In at least some examples, the separator layer 1012 can be comprised of one or more photoresist materials. Additionally, the separator layer 1012 can be comprised of one or more hydrophobic materials and/or one or more low surface energy materials. In one or more illustrative examples, the separator layer 1012 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. The separator layer 1012 can have a thickness from about 1 μm to about 200 μm, from about 5 μm to about 100 μm, from about 10 μm to about 50 μm, from about 50 μm to about 100 μm, from about 1 μm to about 10 μm, from about 10 μm to about 25 μm, from about 50 μm to about 150 μm, or from about 100 μm to about 200 μm. In one or more additional illustrative examples, the separator layer 1012 can have a thickness from about 0.1 μm to about 50 μm, from about 1 μm to about 30 μm, from about 5 μm to about 20 μm, from about 0.1 μm to about 1 μm, from about 0.5 μm to about 2 μm, from about 1 μm to about 5 μm, from about 1 μm to about 10 μm, from about 5 μm to about 15 μm, or from about 5 μm to about 10 μm.

A number of cavities can be formed by the layers of the semiconductor device 1000. In the illustrative example of FIG. 10, the semiconductor device 1000 can include a first cavity 1014, a second cavity 1016, and a third cavity 1018. Side boundaries of the cavities 1014, 1018 can be formed by locations of the polysilicon layer 1006, the one or more passivation layers 1008, the glass substrate 1010, and the separator layer 1012. The second cavity 1016 can have boundaries formed by the semiconductor layer 1002, the functional layer 1004, the polycrystalline layer 1006, the one or more passivation layers 1008, and the glass substrate 1010. Although the illustrative example of FIG. 10 shows three cavities of the semiconductor device 1000, the semiconductor device 1000 can include a greater number of cavities. To illustrate, the semiconductor device 1000 can include at least 10 cavities, at least 50 cavities, at least 100 cavities, at least 500 cavities, at least 1000 cavities, at least 2500 cavities, at least 5000 cavities, at least 10,000 cavities, or more.

In various examples, the second cavity 1016 can correspond to a channel within the semiconductor device 1000 in which one or more liquids can be disposed. In one or more illustrative examples, a spacing between channels of the semiconductor device 1000 can be from about 50 μm to about 800 μm, from about 100 μm to about 600 μm, from about 200 μm to about 500 μm, from about 100 μm to about 300 μm, from about 200 μm to about 400 μm, from about 300 μm to about 500 μm, from about 150 μm to about 250 μm, from about 100 μm to about 200 μm, from about 200 μm to about 300 μm, from about 300 μm to about 400 μm, and from about 400 μm to about 500 μm.

The first cavity 1014 and the third cavity 1018 can store amounts of one or more additional liquids. The one or more additional liquids stored in the first cavity 1014 and the third cavity 1018 can be at least partially different from the liquid disposed on the second cavity 1016. The first cavity 1014 and the third cavity 1018 can have a width 1020 and the second cavity 1016 can have a width 1022. In one or more examples, the widths 1020 and 1022 can be at least substantially the same. In one or more additional examples, the width 1020 and 1022 can be different. In one or more illustrative examples, the widths 1020 and 1022 can be from 25 μm to about 400 μm, from about 50 μm to about 250 μm, from about 100 μm to about 200 μm, from about 25 μm to about 125 μm, from about 50 μm to about 150 μm, from about 50 μm to about 100 μm, or from about 100 μm to about 150 μm. In one or more illustrative examples, the cavities 1014 and 1018 can have a cylindrical shape. In these scenarios, the width 1020 of the cavities 1014 and 1018 can correspond to a diameter.

The semiconductor device 1000 can include a number of pores that are formed by openings between the glass substrate 1010 and the one or more passivation layers 1008. In the illustrative example of FIG. 10, the semiconductor device 1000 can include a first pore 1024 and a second pore 1026. The first pore 1024 can provide fluid communication between the first cavity 1014 and the second cavity 1016. Additionally, the second pore 1026 can provide fluid communication between the third cavity 1018 and the second cavity 1016. Although the illustrative example of FIG. 10 shows two pores of the semiconductor device 1000, the semiconductor device 1000 can include a greater number of pores. For example, the semiconductor device 1000 can include at least 100 pores, at least 500 pores, at least 1000 pores, at least 5000 pores, at least 10,000 pores, at least 25,000 pores, at least 50,000 pores, at least 100,000 pores, or more. The pores 1024, 1026 can have a height 1028 from about 0.1 μm to about 10 μm, from about 0.5 μm to about 5 μm, from about 1 μm to about 3 μm, from about 0.5 μm to about 2 μm, from about 1 μm to about 5 μm, from about 1 μm to about 2 μm, or from about 0.5 μm to about 1.5 μm. The pores 1024, 1026 can also have a length 1030 from about 2 μm to about 20 μm, from about 5 μm to about 15 μm, from about 8 μm to about 12 μm, from about 5 μm to about 10 μm, or from about 10 μm to about 15 μm.

The semiconductor device 1000 can also include a number of sites. The number of sites can be structured to apply electrical stimulation to a discrete amount of liquid located in individual sites. In the illustrative example of FIG. 10, the semiconductor device 1000 can include a first site that corresponds to the first cavity 1014 and the second cavity 1016 and a second site that corresponds to the third cavity 1018 and the second cavity 1016. In this way, at least a portion of the sites of the semiconductor device 1000 can share a common channel corresponding to the second cavity 1016. Although the illustrative example of FIG. 10 shows two sites of the semiconductor device 1000, the semiconductor device 1000 can include a greater number of sites. For example, the semiconductor device 1000 can include at least 100 sites, at least 500 sites, at least 1000 sites, at least 5000 sites, at least 10,000 sites, at least 25,000 sites, at least 50,000 sites, at least 100,000 sites, or more.

The individual sites can each include an electrode. For example, a first electrode 1032 can be disposed in the first cavity 1014 and a second electrode 1034 can be disposed in the second cavity 1018. The electrodes 1032, 1034 can be electrically connected to circuitry of the functional layer 1004. In one or more examples, the first electrode 1032 can be used to apply at least one of voltage or current to liquid disposed in the first cavity 1014 and the second electrode 1034 can be used to apply at least one of voltage or current to liquid disposed in the third cavity 1018. Additionally, the first electrode 1032 can be used to measure signals produced in response to electrical stimulation being provided to liquid disposed in the first cavity 1014 and the second electrode 1034 can be used to measure signals produced in response to electrical stimulation being provided to liquid disposed in the third cavity 1018. The electrodes 1032, 1034 can be comprised of one or more metallic materials. To illustrative, the electrodes 1032, 1034 can be comprised of at least one of sliver chloride, platinum, alloys of platinum, gold, or alloys of gold.

In various examples, the semiconductor device 1000 can also include at least one reference electrode 1036. The at least one reference electrode 1036 can be disposed in the second cavity 1016. The at least one reference electrode 1036 can be comprised of at least one of sliver chloride, platinum, alloys of platinum, gold, or alloys of gold. Although the at least one reference electrode 1036 is shown in the second cavity 1016 in the illustrative example of FIG. 10, in one or more additional implementations, the at least one reference electrode 1036 can be disposed in other channels of the semiconductor device 1000.

In one or more examples, one or more liquids can be disposed in the cavities 1014, 1016, 1018. In one or more illustrative examples, volumes of liquid disposed in the individual cavities 1014, 1016, 1018 can include from about 1 picoliter (pL) to about 100 nanoliters (nL), from about 5 pL to about 10 nL, from about 10 pL to about 1 nL from about 1 pL to about 100 pL, from about 1 pL to about 50 pL, from about 1 pL to about 10 pL, from about 10 pL to about 500 pL, from about 10 pL to about 100 pL, from about 10 pL to about 50 pL, from about 50 pL to about 500 pL, from about 50 pL to about 250 pL, from about 100 pL to about 1 nL, from about 100 pL to about 500 pL, from about 500 pL to about 5 nL, from about 500 pL to about 2 nL, from about 500 pL to about 1 nL, from about 1 nL to about 100 nL, from about 1 nL to about 50 nL, or from about 1 nL to about 10 nL.

In the illustrative example of FIG. 10, a first amount of liquid 1038 can be dispensed into the first cavity 1014 and a second amount of liquid 1040 can be dispensed into the third cavity 1018. Although the illustrative example of FIG. 10 shows the first amount of liquid 1038 being dispensed into the first cavity 1014 and the second amount of liquid 1040 being dispensed into the third cavity 1018, additional amounts of the liquid can also be dispensed into other sites of the semiconductor device 1000. In at least some examples, the first amount of liquid 1038 and the second amount of liquid 1040 can comprise an aqueous solution having one or more biological cells, such as the example biological cell 1042. In various examples, the first amount of liquid 1038 and the second amount of liquid 1040 can be fluidically and electrically isolated from one another. In these situations, independent electrophysiological measurements can be produced for individual sites of the semiconductor device 1000.

In one or more illustrative examples, additional liquid can be present in the second cavity 1016 when the first amount of the liquid 1038 is present in the first cavity 1014 and the second amount of liquid 1040 is present in the third cavity 1018. In addition to biological cells 1042, the first amount of liquid 1038 and the second amount of liquid 1040 can include one or more therapeutic compounds, one or more additional reagents, one or more combinations thereof, and the like. For example, the first amount of liquid 1038 and the second amount of liquid 1040 can include at least one of NaCl, KCl, HEPES buffer, MgCl₂, CaCl₂, glucose, ligands. In one or more additional examples, the additional liquid disposed within the third cavity 1018 can include reagents that are used in relation to patch clamp processes and can include, in at least some examples, one or more therapeutic compounds.

In various examples, obtaining patch clamp measurements can include applying a pressure difference between the first amount of liquid 1038 disposed in the first cavity 1014 and the additional liquid disposed in the second cavity 1016 and between the second amount of liquid disposed in the third cavity 1018 and the additional amount of liquid disposed in the second cavity 1016 to move biological cells 1042 toward the respective pores 1024, 1026. In one or more examples, the biological cells 1042 can form a seal with the pores 1024, 1026 such that the biological cells 1042 are disposed at least partially within the pores 1024, 1026 and against a wall of the cavities 1014, 1018. In at least some examples, a first pressure difference can be applied to move the biological cells 1042 to form seals with the pores 1024, 1026 and a second pressure difference can be applied to cause rupturing of the membranes of the biological cells 1042. In one or more illustrative examples, the pressure difference can correspond to a negative pressure. In still other examples, a single pressure difference can be applied to move the biological cells 1042 toward the respective pores 1024, 1026 and to disrupt the membranes of the biological cells 1042.

While the biological cells 1042 are pressed against the respective walls of the cavities 1014, 1018 using the second amount of pressure to disrupt the membranes of the biological cells 1042, a potential difference can be applied between the electrodes 1032, 1034 and the reference electrode 1036. In these scenarios, voltage clamp measurements can be collected. In one or more additional examples, while the biological cells 1042 are pressed against the respective walls of the cavities 1014, 1018 using the second amount of pressure to disrupt the membranes of the biological cells 1042, the electrodes 1032, 1034 can deliver an amount of current to the biological cells 1042 through the disrupted membranes of the biological cells 1042 to collect current clamp measurements. The measurements collected during the voltage clamp and/or current clamp operations can be used to determine activity of biological cells 1042 with respect to one or more therapeutic compounds and/or to one or more experimental conditions. In various examples, the amount of activity of the biological cells 1042 during patch clamp operations can indicate a responsiveness of the biological cells 1042 to at least one of the one or more therapeutic compounds or the one or more experimental conditions.

In one or more additional illustrative examples, additional biological cells 1044 can be present in the liquid disposed in the second cavity 1016 and are absent from the first cavity 1014 and the third cavity 1018. In these scenarios, the second cavity 1016 can operate as a common channel to provide biological cells for patch clamp measurements. In at least some examples, the amount of liquid disposed in the second cavity 1016 can comprise an aqueous solution having one or more biological cells, such as the example biological cell 1044.

In one or more examples, additional liquid can be present in the first cavity 1014 and the third cavity 1018 when the biological cell-containing liquid is present in the second cavity 1016. In addition to biological cells 1044, the amount of liquid disposed in the second cavity 1016 can include one or more therapeutic compounds, one or more additional reagents, one or more combinations thereof, and the like. In one or more additional examples, the first amount of liquid 1038 disposed within the first cavity 1014 and the second amount of liquid 1040 disposed in the third cavity 1018 can include reagents that are used in relation to patch clamp processes and can include, in at least some examples, one or more therapeutic compounds.

In various examples, obtaining patch clamp measurements can include applying a pressure difference between the amount of liquid disposed in the second cavity 1016 and the first amount of liquid 1038 disposed in the first cavity 1014 and the second amount of liquid disposed in the third cavity 1018 to move biological cells 1044 toward the respective pores 1024, 1026. In one or more examples, the biological cells 1044 can form a seal with the pores 1024, 1026 such that the biological cells 1044 are disposed at least partially within the pores 1024, 1026 and against a wall of the second cavity 1016. In at least some examples, a first pressure difference can be applied to move the biological cells 1044 to form seals with the pores 1024, 1026 and a second pressure difference can be applied to cause rupturing of the membranes of the biological cells 1044. In one or more illustrative examples, the pressure difference can correspond to a negative pressure. In still other examples, a single pressure difference can be applied to move the biological cells 1044 toward the respective pores 1024, 1026 pores and to disrupt the membranes of the biological cells 1044.

While the biological cells 1044 are pressed against the walls of the second cavity 1016 using the second amount of pressure to disrupt the membranes of the biological cells 1044, a potential difference can be applied between the electrodes 1032, 1034 and the reference electrode 1036. In these scenarios, voltage clamp measurements can be collected. In one or more additional examples, while the biological cells 1044 are pressed against the walls of the second cavities 1016 using the second amount of pressure to disrupt the membranes of the biological cells 1044, the electrodes 1032, 1034 can deliver an amount of current to the biological cells 1044 through the disrupted membranes of the biological cells 1044 to collect current clamp measurements. The measurements collected during the voltage clamp and/or current clamp operations can be used to determine activity of biological cells 1044 with respect to one or more therapeutic compounds and/or to one or more experimental conditions. In various examples, the amount of activity of the biological cells 1044 during patch clamp operations can indicate a responsiveness of the biological cells 1044 to at least one of the one or more therapeutic compounds or the one or more experimental conditions.

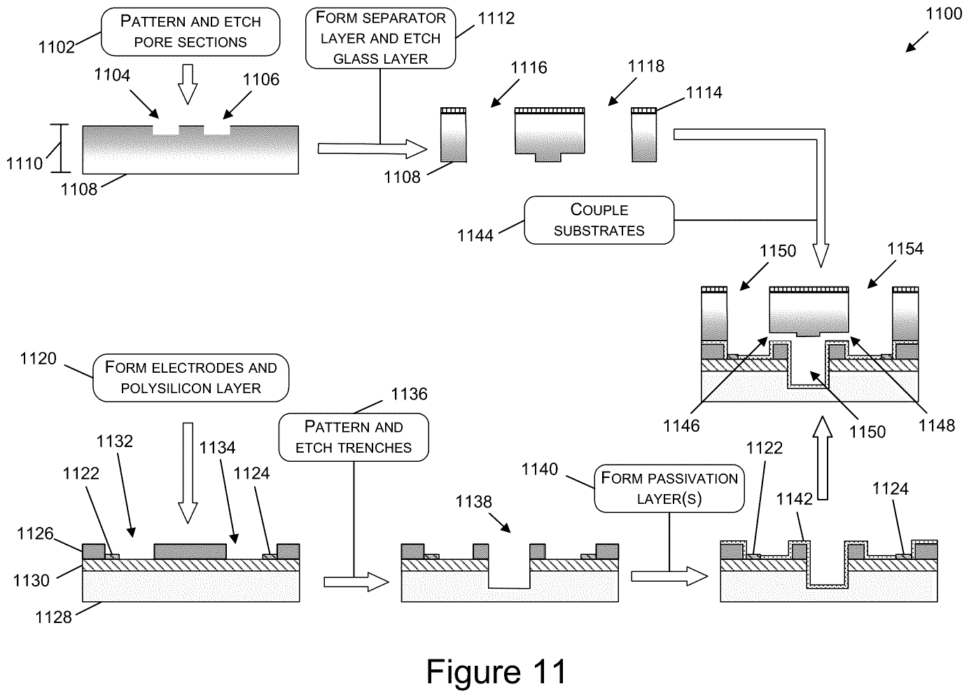

FIG. 11 is a diagram depicting a process 1100 of manufacturing a third example semiconductor device in which at least one of current or voltage can be applied to substances disposed in fluid, in accordance with one or more example implementations. In one or more illustrative examples, the process 1100 can be implemented to produce semiconductor devices having one or more features of the semiconductor device 1000 described in relation to FIG. 10.

The process 1100 can include, at 1102, patterning and etching pore locations 1104, 1106 into a glass substrate 1108. The glass substrate 1108 can have a thickness 1110 from about 50 μm to about 2000 μm, from about 100 μm to about 1500 μm, from about 250 μm to about 1000 μm, from about 100 μm to about 500 μm, from about 500 μm to about 1000 μm, from about 1000 μm to about 1500 μm, from about 100 μm to about 300 μm, from about 200 μm to about 400 μm, from about 300 μm to about 500 μm, from about 400 μm to about 600 μm, from about 500 μm to about 700 μm, from about 600 μm to about 800 μm, from about 700 μm to about 900 μm, or from about 800 μm to about 1000 μm. The pore locations 1104, 1106 can be formed in the glass substrate 1108 using a reactive ion etching process.

The patterning and etching of the pore locations 1104, 1106 can be performed using one or more lithographic processes. The one or more lithographic processes can include depositing a photoresist layer or film on the glass substrate 1108 according to a pattern and then exposing the photoresist to electromagnetic radiation. In scenarios where the photoresist is a positive photoresist, the pore locations 1104, 1106 can be exposed to electromagnetic radiation and the exposed portions of the positive photoresist are removed by the developer solution. In situations where the photoresist is a negative photoresist, the pore locations 1104, 1106 are not exposed to electromagnetic radiation and the unexposed portions of the negative photoresist are removed by the developer solution.

At 1112, the process 1100 can also include forming a separator layer 1114 and etching the glass substrate 1108 to form through-holes 1116, 1118. The separator layer 1114 can be formed from one or more hydrophobic materials. To illustrate, the separator layer 1114 can be formed from one or more photoresist materials. In one or more illustrative examples, the separator layer 1114 can be formed from at least one of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In at least some examples, the separator layer 1114 can be formed using one or more lithographic processes to pattern one or more photoresist materials according to the locations of the through-holes 1116, 1118. In at least some examples, the separator layer 1114 can be formed using a spin coating process. Additionally, the through-holes 1116, 1118 can be formed using one or more anisotropic etching processes. To illustrate, the through-holes 1116, 1118 can be formed using at least one of laser drilling processes or mechanical milling processes.

In addition, the process 1100 can include, at 1120, forming electrodes 1122, 1124 and a polycrystalline silicon layer 1126 on a semiconductor substrate. The semiconductor substrate can include a semiconductor layer 1128 and a functional layer 1130. The first electrode 1122 can be formed in a first cavity location 1132 and the second electrode 1124 can be formed in a second cavity location 1134. The electrodes 1122, 1124 and the polycrystalline silicon layer 1126 can be formed by one or more lithographic processes. In one or more examples, the electrodes 1122, 1124 can be formed by at least one of electroplating, sputter deposition, chemical vapor deposition, or physical vapor deposition. The electrodes 1122, 1124 can be comprised of one or more metals that can be used to pass current through an aqueous liquid. In one or more examples, the electrodes 1122, 1124 can be comprised of at least one of silver chloride, platinum, alloys of platinum, gold, or alloys of gold. The polycrystalline silicon layer 1126 can be formed on the semiconductor substrate using one or more low pressure chemical vapor deposition processes.

In one or more examples, the semiconductor layer 1128 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon. The functional layer 1130 can include circuitry formed on one or more substrates. In one or more examples, the functional layer 1130 can include circuitry to apply at least one of voltage or current to liquid and to measure signals produced in response to the electrical stimulation provided to the liquid. In one or more illustrative examples, the functional layer 1130 can include circuitry to perform at least one of voltage clamp operations, current clamp operations, or dynamic clamp operations.

Further, the process 1100 can include, at 1136 patterning and etching trenches, such as example trench 1138, in the polycrystalline silicon layer 1126, in the semiconductor layer 1128, and in the functional layer 1130. The trench 1138 can be formed using one or more lithographic processes. In one or more illustrative examples, the trench 1138 can be formed using one or more deep reactive ion etching processes.

At 1140, the process 1100 can include forming one or more passivation layers 1142 on the electrodes 1122, 1124, on the polycrystalline silicon layer 1126, on the functional layer 1130, and within the trench 1138. The one or more passivation layers 1142 can be comprised of at least one of one or more resistive materials. For example, the one or more passivation layers 1142 can be comprised of at least one of one or more oxides or one or more nitrides. To illustrate, the one or more passivation layers 1142 can be comprised of at least one of silicon oxide or silicon nitride. In scenarios where the one or more passivation layers 1142 include an oxide material, the one or more oxide-containing passivation layers can be formed by at least one of thermal oxidation, chemical vapor deposition, physical vapor deposition, plasma enhanced chemical vapor deposition, thermal evaporation, or electron beam evaporation. Additionally, in situations where the one or more passivation layers 1142 include a nitride material, the one or more nitride-containing passivation layers can be formed by at least one of low pressure chemical vapor deposition, plasma enhanced chemical vapor deposition, thermal atomic layer deposition, or plasma enhanced atomic layer deposition. The one or more passivation layers 1142 can have a thickness from about 0.1 μm to about 5 μm, from about 0.5 μm to about 4 μm, from about 1 μm to about 3 μm, from about 0.1 μm to about 1 μm, from about 1 μm to about 2 μm, from about 2 μm to about 3 μm, from about 10 nm to about 1 μm, from about 50 nm to about 800 nm, from about 100 nm to about 500 nm, from about 500 nm to about 1 μm, from about 50 nm to about 200 nm, from about 100 nm to about 300 nm, from about 200 nm to about 400 nm, or from about 300 nm to about 500 nm. In at least some examples, the one or more passivation layers 1142 can be removed from the electrodes 1122, 1124 via one or more etching processes.

The process 1100 can also include, at 1144, coupling the patterned and etched glass substrate having the through-holes 1116, 1118 with the semiconductor substrate on which the electrodes 1122, 1124, polycrystalline silicon layer 1126, and the one or more passivation layers 1142 are formed. The coupling of the substrates at 1144 can produce pores 1146, 1148 and cavities 1150, 1152, and 1154. In one or more examples, the substrates can be coupled at 1144 using direct wafer bonding techniques. In one or more additional illustrative examples, the substrates can be coupled using mediated wafer bonding techniques.

Although the illustrative example of FIG. 11 describes the formation of features of a one or two sites of a semiconductor device, the process 1100 can be used to form features of a greater number of sites of a semiconductor device. For example, the process 1100 can be used to form features of at least 5 sites, at least 20 sites, at least 50 sites, at least 100 sites, at least 200 sites, at least 500 sites, at least 1000 sites, at least 2000 sites, at least 5000 sites, at least 8000 sites, at least 10,000 sites, at least 25,000 sites, at least 50,000 sites, at least 75,000 sites, at least 100,000 sites, or more. Additionally, although the process 1100 has been described in relation to a polycrystalline silicon layer 1126 being formed on the semiconductor substrate, in one or more additional examples, the polycrystalline silicon layer 1126 can be omitted.

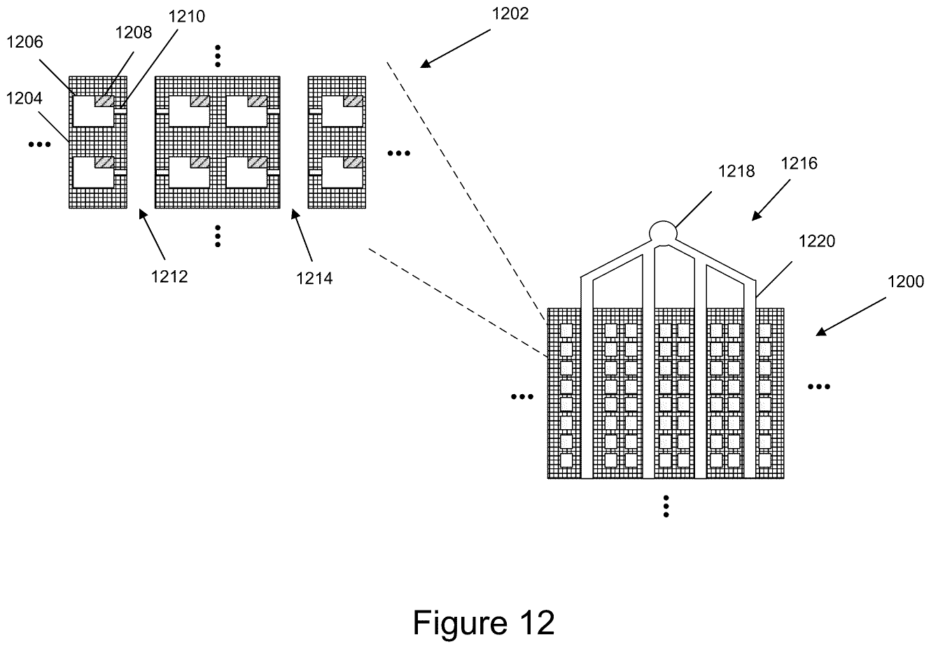

FIG. 12 is a diagram depicting a top view of a third example semiconductor device having an array of sites 1200 in which at least one of current or voltage can be applied to substances disposed in fluid, in accordance with one or more example implementations. In one or more examples, the array of sites 1200 can include sites that correspond to sites of the semiconductor device 1000 described in relation to FIG. 10 and the semiconductor device manufactured according to the process 1100 described in relation to FIG. 11. In one or more examples, the array of sites 1200 can have tens of sites, hundreds of sites, thousands of sites, up to tens of thousands of sites, or more.

A subsection 1202 of the array of sites 1200 includes a number of individual sites and a number of features of the individual sites. For example, the subsection 1202 can include a separator layer 1204 in which a plurality of cavities are disposed within the separator layer 1204 as well as disposed in layers beneath the separator layer 1204, such as a semiconductor layer, a functional layer, a glass substrate, and/or a polycrystalline silicon layer. For example, cavities, such as example cavity 1206, can be included in the subsection 1202 with electrodes, such as an example electrode 1208, disposed within the cavities. Additionally, channels 1212 and 1214 can be formed in the subsection 1202. Pores, such as example pore 1210, can couple the channels 1212, 1214 to electrode cavities such that the electrode cavities are in fluid communication with the channels 1212, 1214. In one or more illustrative examples, the cavity 1206 can correspond to first cavity 1014, the channel 1212 can correspond to the second cavity 1016, and the pore 1210 can correspond to the pore 1024 described in relation to FIG. 10.

FIG. 12 also illustrates an example fluid distribution system 1216. The fluid distribution system 1216 can include a fluidic inlet port 1218 that receives fluid from one or more fluid sources. The fluid distribution system 1216 and the one or more fluid sources can correspond to a fluid delivery system. The fluid distribution system 1216 can also include a number of channels 1220. The number of channels 1220 can include the channels 1212, 1214 of the subsection 1201. Although not shown in the illustrative example of FIG. 12, flow of liquid from the fluidic inlet port 1218 can be controlled by valves disposed between the fluidic inlet port 1218 and the electrode cavities. In one or more illustrative examples, the valves can be included in the fluid distribution system 1216 in scenarios where biological cells are disposed in the channels 1220.

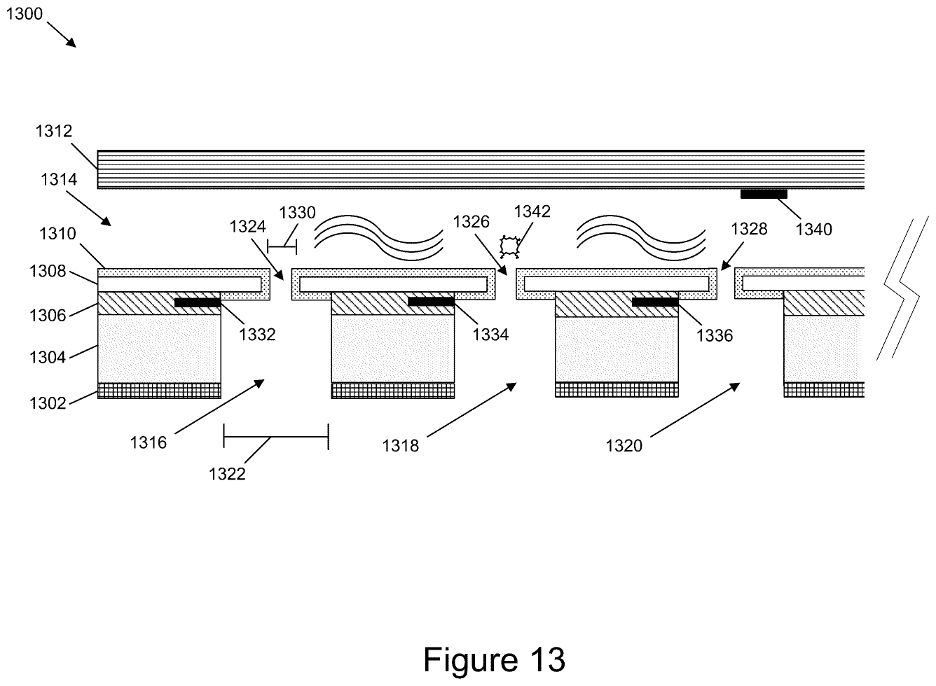

FIG. 13 is a diagram depicting a cross-section of at least a portion of a fourth example semiconductor device 1300 in which at least one of current or voltage can be applied to substances disposed in fluid, in accordance with one or more example implementations. In at least some examples, the first example semiconductor device 1300 can correspond to an implementation of the semiconductor device 102 described in relation to FIG. 1.

The semiconductor device 1300 can include a separator layer 1302. The separator layer 1302 can isolate portions of the semiconductor device 1300 in which one or more liquids are disposed. The separator layer 1302 can be comprised of one or more polymeric materials. In at least some examples, the separator layer 1302 can be comprised of one or more photoresist materials. Additionally, the separator layer 1302 can be comprised of one or more hydrophobic materials and/or one or more low surface energy materials. In one or more illustrative examples, the separator layer 1302 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. The separator layer 1302 can have a thickness from about 0.1 μm to about 100 μm, from about 1 μm to about 50 μm, from about 5 μm to about 30 μm, from about 0.1 μm to about 10 μm, from about 0.5 μm to about 8 μm, from about 1 μm to about 5 μm, from about 1 μm to about 10 μm, from about 5 μm to about 15 μm, from about 5 μm to about 10 μm, from about 10 μm to about 20 μm, from about 15 μm to about 25 μm, from about 20 μm to about 30 μm, from about 25 μm to about 35 μm, from about 30 μm to about 40 μm, or from about 40 μm to about 50 μm.

The semiconductor device 1300 can also include a semiconductor layer 1304. The semiconductor layer 1304 can include a silicon-containing layer. In one or more examples, the semiconductor layer 1304 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

In addition, the semiconductor device 1300 can include a functional layer 1306 disposed on the semiconductor layer 1304. The functional layer 1306 can include circuitry formed on one or more substrates. In one or more examples, the functional layer 1306 can include circuitry to apply at least one of voltage or current to liquid disposed on the semiconductor device 1300 and to measure signals produced in response to the electrical stimulation provided to the liquid. In one or more illustrative examples, the functional layer 1306 can include circuitry to perform at least one of voltage clamp operations, current clamp operations, or dynamic clamp operations. In various examples, the functional layer 1306 can include analog-to-digital converters (ADCs), digital-to-analog converters (DACs), amplifiers, digital signal processing circuitry, one or more combinations thereof, and the like. The functional layer 1306 can also include routing circuitry to carry electrical signals to locations on a die and/or on packaging of the semiconductor device 1300 for further processing. The circuitry of the functional layer 1306 can be produced using CMOS technologies.

Further, the semiconductor device 1300 can include one or more passivation layers 1308 disposed on the functional layer 1306. The one or more passivation layers 1308 can be comprised of one or more oxide materials and/or one or more nitride materials. For example, the one or more passivation layers 1308 can be comprised of at least one of silicon oxide, silicon nitride, aluminum oxide, or titanium oxide. In one or more additional examples, the one or more passivation layers 1308 can be comprised of polycrystalline silicon. In various examples, the one or more passivation layers 1308 can be comprised of polycrystalline silicon doped with at least one of arsenic, phosphorus, or boron. The one or more passivation layers 1308 can also be comprised of undoped polycrystalline silicon. The one or more passivation layers 1308 can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

One or more coatings 1310 can be disposed over outer surfaces of the semiconductor layer 1304, the functional layer 1306, and the one or more passivation layers 1308. The one or more coatings 1310 can include one or more resistive materials. To illustrate, the one or more coatings 1310 can include one or more oxide materials or one or more nitride materials. For example, the one or more coatings 1310 can comprise at least one of silicon oxide, silicon nitride, or aluminum oxide. The one or more coatings 1310 can have a thickness from about 0.01 μm to about 3 μm, from about 0.05 μm to about 2 μm, from about 0.1 μm to about 1.5 μm, from about 0.01 μm to about 0.1 μm, from about 0.01 μm to about 0.05 μm, from about 0.1 μm to about 1 μm, from about 0.05 μm to about 0.5 μm, or from about 1 μm to about 2 μm.

The semiconductor device 1300 can also include an additional substrate 1312 that can be a lid for the semiconductor device 1300. The additional substrate 1312 can be comprised of one or more polymeric materials or one or more semiconductor materials. A fluid chamber 1314 can be formed between the additional substrate 1312 and the coated one or more passivation layers 1308. Liquid can be disposed in the fluid chamber 1314. In various examples, the liquid can be flowing through the fluid chamber 1314. Thus, in at least some examples, the liquid disposed in the fluid chamber 1314 is not static for at least a period of time.

A number of cavities can be formed by the layers of the semiconductor device 1300. In one or more examples, the number of cavities can be in fluid communication with the fluid chamber 1314. In the illustrative example of FIG. 13, the semiconductor device 1300 can include a first cavity 1316, a second cavity 1318, and a third cavity 1320. Side boundaries of the cavities 1316, 1318, 1320 can be formed by locations of the semiconductor layer 1304, the functional layer 1306, the one or more passivation layers 1308, and the one or more coatings 1310. Although the illustrative example of FIG. 13 shows three cavities of the semiconductor device 1300, the semiconductor device 1300 can include a greater number of cavities. To illustrate, the semiconductor device 1300 can include at least 10 cavities, at least 50 cavities, at least 100 cavities, at least 500 cavities, at least 1000 cavities, at least 2500 cavities, at least 5000 cavities, at least 10,000 cavities, or more. In various examples, the cavities 1316, 1318, 1320 can form channels within the semiconductor device 1300 in which one or more liquids can be disposed. The cavities 1316, 1318, 1320 can have a width 1322. The width 1322 of the cavities 1316, 1318, 1320 can be from 25 μm to about 400 μm, from about 50 μm to about 250 μm, from about 100 μm to about 200 μm, from about 25 μm to about 125 μm, from about 50 μm to about 150 μm, from about 50 μm to about 100 μm, or from about 100 μm to about 150 μm. In one or more illustrative examples, the cavities 1316, 1318, 1320 can have a cylindrical shape. In these scenarios, the width 1322 of the cavities 1316, 1318, 1320 can correspond to a diameter.

The semiconductor device 1300 can include a number of pores that are formed by openings in the one or more passivation layers 1308 and the one or more coatings 1310. In the illustrative example of FIG. 13, the semiconductor device 1300 can include a first pore 1324, a second pore 1326, and third pore 1328. Although the illustrative example of FIG. 13 shows three pores of the semiconductor device 1300, the semiconductor device 1300 can include a greater number of pores. For example, the semiconductor device 1300 can include at least 100 pores, at least 500 pores, at least 1000 pores, at least 5000 pores, at least 10,000 pores, at least 25,000 pores, at least 50,000 pores, at least 100,000 pores, or more. The pores 1324, 1326, 1328 can have a width 1330 from about 0.1 μm to about 25 μm, from about 1 μm to about 20 μm, from about 5 μm to about 15 μm, from about 1 μm to about 10 μm, from about 10 μm to about 20 μm, from about 1 μm to about 5 μm, from about 0.1 μm to about 1 μm, from about 0.5 μm to about 2 μm, or from about 5 μm to about 10 μm.

Further, a spacing between the pores 1324, 1326, 1328 can comprise a pitch of the semiconductor device 1300. In one or more examples, the pitch of the semiconductor device 1300 can be measured from a center of the first pore 1324 to a center of the second pore 1326. The pitch of the semiconductor device 1300 can also be measured from a center of the second pore 1326 to a center of the third pore 1328. In at least some examples, the distance from a center of the first pore 1324 to a center of the second pore 1326 can be at least substantially the same as the distance from a center of the second pore 1326 to a center of the third pore 1328. In one or more illustrative examples, a pitch of the semiconductor device 1300 can be from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

The semiconductor device 1300 can also include a number of electrodes. The semiconductor device 1300 can include a first electrode 1332 disposed in the first cavity 1316, a second electrode 1334 disposed in the second cavity 1318, and a third electrode 1336 disposed in the third cavity 1320. The electrodes 1332, 1334, 1336 can be electrically connected to circuitry of the functional layer 1306. In one or more examples, the first electrode 1332, the second electrode 1334, and the third electrode 1336 can be used to apply at least one of voltage or current to liquid disposed in the cavities 1316, 1318, 1320. Additionally, at least one of the first electrode 1332, the second electrode 1334, and the third electrode 1336 can be used to measure signals produced in response to electrical stimulation being provided to liquid disposed in the cavities 1316, 1318, 1320. The electrodes 1332, 1334, 1336 can be comprised of one or more metallic materials. To illustrative, the electrodes 1332, 1334, 1336 can be comprised of at least one of sliver chloride, platinum, alloys of platinum, gold, or alloys of gold.

In various examples, the semiconductor device 1300 can also include at least one reference electrode 1340. The at least one reference electrode 1340 can be disposed on a surface of the additional substrate 1312 and located in the fluid chamber 1314. The at least one reference electrode 1340 can be comprised of at least one of sliver chloride, platinum, alloys of platinum, gold, or alloys of gold.

The cavities 1316, 1318, 1320, the pores 1324, 1326, 1328, and the respective electrodes 1332, 1334, 1336 can comprise sites of the semiconductor device 1300. The number of sites can be structured to apply electrical stimulation to a discrete amount of liquid located in individual sites. In the illustrative example of FIG. 13, the semiconductor device 1300 includes a first site comprised of the first cavity 1316, the first pore 1324, and the first electrode 1332; a second site comprised of the second cavity 1318, the second pore 1326, and the first electrode 1334; and a third site comprised of the third cavity 1320, the third pore 1328, and the third electrode 1336. The sites can also be defined by sections of the separator layer 1302 that fluidically and electrically isolate liquid disposed in the sites of the semiconductor device 1300. Although the illustrative example of FIG. 13 shows three sites of the semiconductor device 1300, the semiconductor device 1300 can include a greater number of sites. For example, the semiconductor device 1300 can include at least 100 sites, at least 500 sites, at least 1000 sites, at least 5000 sites, at least 10,000 sites, at least 25,000 sites, at least 50,000 sites, at least 100,000 sites, or more.

In one or more examples, a volume of liquid can be stored in the cavities 1316, 1318, 1320. In at least some examples, the fluid disposed within the cavities 1316, 1318, 1320 can include reagents that are used in relation to patch clamp processes and can include, in at least some examples, one or more therapeutic compounds. For example, the fluid disposed in the cavities 1316, 1318, 1320 can include at least one of NaCl, KCl, HEPES buffer, MgCl₂, CaCl₂, glucose, ligands. Additionally, liquid disposed in the fluid chamber 1314 can include biological cells, such as example biological cell 1342, one or more therapeutic compounds, one or more additional reagents, one or more combinations thereof, and the like.

In various examples, obtaining patch clamp measurements can include applying a pressure difference between the fluid disposed in the fluid chamber 1314 and the fluid disposed in the cavities 1316, 1318, 1320 to move biological cells 1342 toward the respective pores 1324, 1326, 1328. In one or more examples, the biological cells 1342 can form a seal with the pores 1324, 1326, 1328 such that the biological cells 1342 are disposed at least partially within the pores 1324, 1326, 1328 and on the portions of the coated passivation layer(s) disposed around the pores 1324, 1326, 1328. In at least some examples, a first pressure difference can be applied to move the biological cells 1342 to form seals with the pores 1324, 1326, 1328 and a second pressure difference can be applied to cause rupturing of the membranes of the biological cells 1342. In one or more illustrative examples, the pressure difference can correspond to a negative pressure. In still other examples, a single pressure difference can be applied to move the biological cells 1342 toward the respective pores 1324, 1326, 1328 and to disrupt the membranes of the biological cells 1342.

While the biological cells 1342 are pressed against the coated one or more passivation layers 1308 using the second amount of pressure to disrupt the membranes of the biological cells 1342, a potential difference can be applied between the electrodes 1332, 1334, 1336, and the reference electrode 1340. In these scenarios, voltage clamp measurements can be collected. In one or more additional examples, while the biological cells 1342 are pressed against the coated one or more passivation layers 1308 using the second amount of pressure to disrupt the membranes of the biological cells 1342, the electrodes 1332, 1334, 1336 can deliver an amount of current to the biological cells 1342 through the disrupted membranes of the biological cells 1342 to collect current clamp measurements. The measurements collected during the voltage clamp and/or current clamp operations can be used to determine activity of biological cells 1342 with respect to one or more therapeutic compounds and/or to one or more experimental conditions. In various examples, the amount of activity of the biological cells 1342 during patch clamp operations can indicate a responsiveness of the biological cells 1342 to at least one of the one or more therapeutic compounds or the one or more experimental conditions.

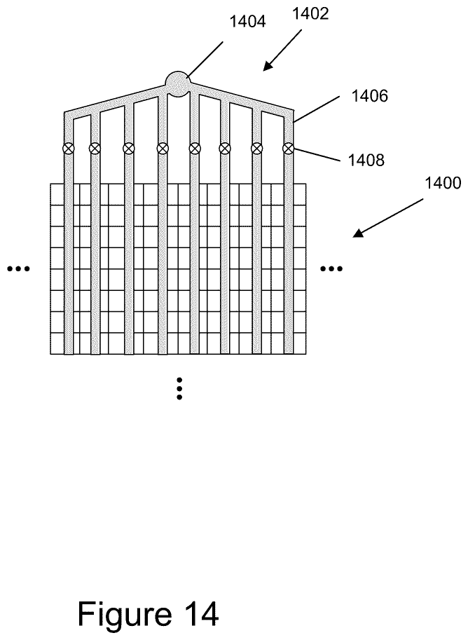

FIG. 14 is a top view of a fourth example semiconductor device having an array of sites 1400 in which at least one of current or voltage can be applied to substances disposed in fluid, in accordance with one or more example implementations. In one or more examples, the array of sites 1400 can include sites that correspond to sites of the semiconductor device 1300 described in relation to FIG. 13. In one or more examples, the array of sites 1400 can have tens of sites, hundreds of sites, thousands of sites, up to tens of thousands of sites, or more.

FIG. 14 also illustrates an example fluid distribution system 1402. The fluid distribution system 1402 can include a fluidic inlet port 1404 that receives fluid from one or more fluid sources. The fluid distribution system 1402 and the one or more fluid sources can correspond to a fluid delivery system. The fluid distribution system 1402 can also include a number of channels 1406. At least a portion of the number of channels 1406 can correspond to the cavities 1316, 1318, 1320 described in relation to the semiconductor device 1300 described in relation to FIG. 13. The fluid distribution system 1402 can also include a number of valves 1408 that control the flow of liquid from the fluidic inlet port 1404 to the individual channels. In these instances, the valves 1408 can be selectively activated to provide different liquids to different channels 1406. In this way, in at least some examples, the effects of different therapeutic compounds on biological cells can be measured.

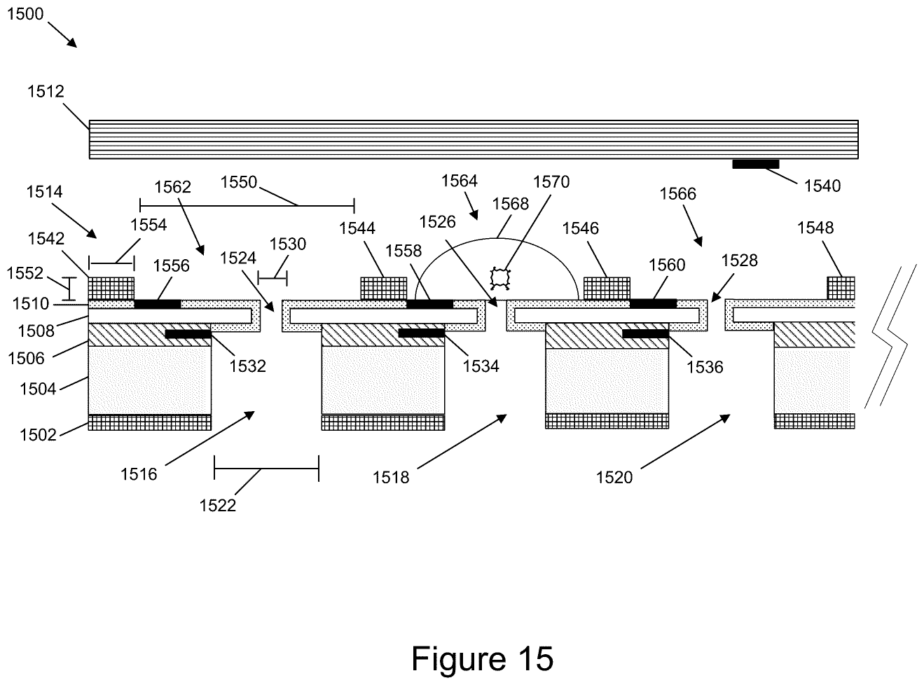

FIG. 15 is a diagram depicting a cross-section of at least a portion of a first example semiconductor device 1500 in which at least one of current or voltage can be applied to substances disposed in fluid, in accordance with one or more example implementations. In at least some examples, the first example semiconductor device 1500 can correspond to an implementation of the semiconductor device 102 described in relation to FIG. 1.

The semiconductor device 1500 can include a separator layer 1502. The separator layer 1502 can isolate portions of the semiconductor device 1500 in which one or more liquids are disposed. The separator layer 1502 can be comprised of one or more polymeric materials. In at least some examples, the separator layer 1502 can be comprised of one or more photoresist materials. Additionally, the separator layer 1502 can be comprised of one or more hydrophobic materials and/or one or more low surface energy materials. In one or more illustrative examples, the separator layer 1502 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. The separator layer 1502 can have a thickness from about 0.1 μm to about 100 μm, from about 1 μm to about 50 μm, from about 5 μm to about 30 μm, from about 0.1 μm to about 10 μm, from about 0.5 μm to about 8 μm, from about 1 μm to about 5 μm, from about 1 μm to about 10 μm, from about 5 μm to about 15 μm, from about 5 μm to about 10 μm, from about 10 μm to about 20 μm, from about 15 μm to about 25 μm, from about 20 μm to about 30 μm, from about 25 μm to about 35 μm, from about 30 μm to about 40 μm, or from about 40 μm to about 50 μm.

The semiconductor device 1500 can also include a semiconductor layer 1504. The semiconductor layer 1504 can include a silicon-containing layer. In one or more examples, the semiconductor layer 1504 can be comprised of at least 50% by weight silicon, at least 60% by weight silicon, at least 70% by weight silicon, at least 80% by weight silicon, at least 90% by weight silicon, at least 95% by weight silicon, or at least 99% by weight silicon.

In addition, the semiconductor device 1500 can include a functional layer 1506 disposed on the semiconductor layer 1504. The functional layer 1506 can include circuitry formed on one or more substrates. In one or more examples, the functional layer 1306 can include circuitry to apply at least one of voltage or current to liquid disposed on the semiconductor device 1500 and to measure signals produced in response to the electrical stimulation provided to the liquid. In one or more illustrative examples, the functional layer 1506 can include circuitry to perform at least one of voltage clamp operations, current clamp operations, or dynamic clamp operations. In various examples, the functional layer 1506 can include analog-to-digital converters (ADCs), digital-to-analog converters (DACs), amplifiers, digital signal processing circuitry, one or more combinations thereof, and the like. The functional layer 1506 can also include routing circuitry to carry electrical signals to locations on a die and/or on packaging of the semiconductor device 1500 for further processing. The circuitry of the functional layer 1506 can be produced using CMOS technologies.

Further, the semiconductor device 1500 can include one or more passivation layers 1508 disposed on the functional layer 1506. The one or more passivation layers 1508 can be comprised of one or more oxide materials and/or one or more nitride materials. For example, the one or more passivation layers 1508 can be comprised of at least one of silicon oxide, silicon nitride, aluminum oxide, or titanium oxide. In one or more additional examples, the one or more passivation layers 1508 can be comprised of polycrystalline silicon. In various examples, the one or more passivation layers 1508 can be comprised of polycrystalline silicon doped with at least one of arsenic, phosphorus, or boron. The one or more passivation layers 1508 can also be comprised of undoped polycrystalline silicon. The one or more passivation layers 1508 can have thickness from about 0.5 micrometers (μm) to about 25 μm, from about 1 μm to about 20 μm, from about 2 μm to about 15 μm, from about 1 μm to about 10 μm, from about 5 μm to about 15 μm, from about 10 μm to about 20 μm, from about 1 μm to about 5 μm, from about 5 μm to about 10 μm, 10 μm to about 15 μm, from about 2 μm to about 8 μm, or from about 4 μm to about 10 μm.

One or more coatings 1510 can be disposed over outer surfaces of the semiconductor layer 1504, the functional layer 1506, and the one or more passivation layers 1508. The one or more coatings 1510 can include one or more resistive materials. To illustrate, the one or more coatings 1510 can include one or more oxide materials or one or more nitride materials. For example, the one or more coatings 1510 can comprise at least one of silicon oxide, silicon nitride, or aluminum oxide. The one or more coatings 1510 can have a thickness from about 0.01 μm to about 3 μm, from about 0.05 μm to about 2 μm, from about 0.1 μm to about 1.5 μm, from about 0.01 μm to about 0.1 μm, from about 0.01 μm to about 0.05 μm, from about 0.1 μm to about 1 μm, from about 0.05 μm to about 0.5 μm, or from about 1 μm to about 2 μm.

The semiconductor device 1500 can also include an additional substrate 1512 that can be a lid for the semiconductor device 1500. The additional substrate 1512 can be comprised of one or more polymeric materials or one or more semiconductor materials. A fluid chamber 1514 can be formed between the additional substrate 1512 and the coated one or more passivation layers 1508. Liquid can be disposed in the fluid chamber 1514. In various examples, the liquid can be flowing through the fluid chamber 1514. Thus, in at least some examples, the liquid disposed in the fluid chamber 1514 is not static for at least a period of time.

A number of cavities can be formed by the layers of the semiconductor device 1500. In one or more examples, the number of cavities can be in fluid communication with the fluid chamber 1514. In the illustrative example of FIG. 15, the semiconductor device 1500 can include a first cavity 1516, a second cavity 1518, and a third cavity 1520. Side boundaries of the cavities 1516, 1518, 1520 can be formed by locations of the semiconductor layer 1504, the functional layer 1506, the one or more passivation layers 1508, and the one or more coatings 1510. Although the illustrative example of FIG. 15 shows three cavities of the semiconductor device 1500, the semiconductor device 1500 can include a greater number of cavities. To illustrate, the semiconductor device 1500 can include at least 10 cavities, at least 50 cavities, at least 100 cavities, at least 500 cavities, at least 1000 cavities, at least 2500 cavities, at least 5000 cavities, at least 10,000 cavities, or more. In various examples, the cavities 1516, 1518, 1520 can form channels within the semiconductor device 1500 in which one or more liquids can be disposed. The cavities 1516, 1518, 1520 can have a width 1522. The width 1522 of the cavities 1516, 1518, 1520 can be from 25 μm to about 400 μm, from about 50 μm to about 250 μm, from about 100 μm to about 200 μm, from about 25 μm to about 125 μm, from about 50 μm to about 150 μm, from about 50 μm to about 100 μm, or from about 100 μm to about 150 μm. In one or more illustrative examples, the cavities 1516, 1518, 1520 can have a cylindrical shape. In these scenarios, the width 1322 of the cavities 1516, 1518, 1520 can correspond to a diameter.

The semiconductor device 1500 can include a number of pores that are formed by openings in the one or more passivation layers 1508 and the one or more coatings 1510. In the illustrative example of FIG. 15, the semiconductor device 1500 can include a first pore 1524, a second pore 1526, and third pore 1528. Although the illustrative example of FIG. 15 shows three pores of the semiconductor device 1500, the semiconductor device 1500 can include a greater number of pores. For example, the semiconductor device 1500 can include at least 100 pores, at least 500 pores, at least 1000 pores, at least 5000 pores, at least 10,000 pores, at least 25,000 pores, at least 50,000 pores, at least 100,000 pores, or more. The pores 1524, 1526, 1528 can have a width 1530 from about 0.1 μm to about 25 μm, from about 1 μm to about 20 μm, from about 5 μm to about 15 μm, from about 1 μm to about 10 μm, from about 10 μm to about 20 μm, from about 1 μm to about 5 μm, from about 0.1 μm to about 1 μm, from about 0.5 μm to about 2 μm, or from about 5 μm to about 10 μm.

Further, a spacing between the pores 1524, 1526, 1528 can comprise a pitch of the semiconductor device 1500. In one or more examples, the pitch of the semiconductor device 1500 can be measured from a center of the first pore 1524 to a center of the second pore 1526. The pitch of the semiconductor device 1500 can also be measured from a center of the second pore 1526 to a center of the third pore 1528. In at least some examples, the distance from a center of the first pore 1524 to a center of the second pore 1526 can be at least substantially the same as the distance from a center of the second pore 1526 to a center of the third pore 1528. In one or more illustrative examples, a pitch of the semiconductor device 1500 can be from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

The semiconductor device 1500 can also include a number of electrodes. The semiconductor device 1500 can include a first electrode 1532 disposed in the first cavity 1516, a second electrode 1534 disposed in the second cavity 1518, and a third electrode 1536 disposed in the third cavity 1520. The electrodes 1532, 1534, 1536 can be electrically connected to circuitry of the functional layer 1506. In one or more examples, the first electrode 1532, the second electrode 1534, and the third electrode 1536 can be used to apply at least one of voltage or current to liquid disposed in the cavities 1516, 1518, 1520. Additionally, at least one of the first electrode 1532, the second electrode 1534, and the third electrode 1536 can be used to measure signals produced in response to electrical stimulation being provided to liquid disposed in the cavities 1516, 1518, 1520. The electrodes 1532, 1534, 1536 can be comprised of one or more metallic materials. To illustrative, the electrodes 1532, 1534, 1536 can be comprised of at least one of sliver chloride, platinum, alloys of platinum, gold, or alloys of gold.

In various examples, the semiconductor device 1500 can also include at least one reference electrode 1540. The at least one reference electrode 1540 can be disposed on a surface of the additional substrate 1512 and located in the fluid chamber 1514. The at least one reference electrode 1540 can be comprised of at least one of sliver chloride, platinum, alloys of platinum, gold, or alloys of gold.

The semiconductor device 1500 can also include a number of walls disposed on the coated one or more passivation layers 1510. For example, the semiconductor device 1500 can include a first wall 1542, a second wall 1544, a third wall 1546, and a fourth wall 1548. The walls 1542, 1544, 1546, 1548 can define wells in which an amount of liquid can be disposed. Although not shown in the illustrative cross sectional view of FIG. 15, in one or more examples, wells of the semiconductor device 1500 can also be defined by walls disposed in rows perpendicular to the walls 1542, 1544, 1546, 1548. In this way, the wells can be defined by a rectangular shape having boundaries formed by four walls. The wells can have an inner width 1550 from about 50 μm to about 500 μm, from about 100 μm to about 400 μm, from about 200 μm to about 300 μm, from about 100 μm to about 300 μm, from about 200 μm to about 400 μm, from about 50 μm to about 150 μm, from about 150 μm to about 250 μm, from about 100 μm to about 200 μm, or from about 200 μm to about 300 μm.

In one or more examples, the walls 1542, 1544, 1546, 1548 can be comprised of one or more polymeric materials. In at least some examples, the walls 1542, 1544, 1546, 1548 can be comprised of one or more photoresist materials. Additionally, the walls 1542, 1544, 1546, 1548 can be comprised of one or more hydrophobic materials and/or one or more low surface energy materials. In one or more illustrative examples, the walls 1542, 1544, 1546, 1548 can be comprised of an SU-8 photoresist, polytetrafluoroethylene (PTFE), one or more polyimides, one or more epoxy-based polymers, benzocyclobutene (BCB), polydimethylsiloxane (PDMS), polymethylmethacrylate (PMMA), or one or more silanes. The one or more silanes can include octadecyltrimethoxysilane. In one or more illustrative examples, the walls 1542, 1544, 1546, 1548 can have a height 1552 from about 1 μm to about 200 μm, from about 5 μm to about 100 μm, from about 10 μm to about 50 μm, from about 50 μm to about 100 μm, from about 1 μm to about 10 μm, from about 10 μm to about 25 μm, from about 50 μm to about 150 μm, or from about 100 μm to about 200 μm. In one or more additional illustrative examples, the walls 1542, 1544, 1546, 1548 can have a thickness 1554 from about 0.1 μm to about 50 μm, from about 1 μm to about 30 μm, from about 5 μm to about 20 μm, from about 0.1 μm to about 1 μm, from about 0.5 μm to about 2 μm, from about 1 μm to about 5 μm, from about 1 μm to about 10 μm, from about 5 μm to about 15 μm, or from about 5 μm to about 10 μm.

The individual wells can each include an electrode. The electrodes included in the individual wells can be electrically connected to circuitry of the functional layer 1506. In the illustrative example of FIG. 15, a first well electrode 1556 can be located in a first well 1562 defined by the walls 1542, 1544; a second well electrode 1558 can be located in a second well 1564 defined by the walls 1544, 1546; and a third well electrode 1560 can be located in a third well 1566 defined by walls 1546, 1548. In one or more examples, the first well electrode 1556, the second well electrode 1558, and the third well electrode 1560 can be used to apply at least one of voltage or current to liquid disposed in the respective wells 1562, 1564, 1566. Additionally, at least one of the first well electrode 1556, the second well electrode 1558, and the third well electrode 1560 can be used to measure signals produced in response to electrical stimulation being provided to liquid disposed in the respective wells 1562, 1564, 1566. The electrodes 1556, 1558, 1560 can be comprised of one or more metallic materials. To illustrative, the electrodes 1556, 1558, 1560 can be comprised of at least one of sliver chloride, platinum, alloys of platinum, gold, or alloys of gold.

The cavities 1516, 1518, 1520, the pores 1524, 1526, 1528, the respective electrodes 1532, 1534, 1536, 1556, 1558, 1560, and the wells 1562, 1564, 1566 can comprise sites of the semiconductor device 1500. The number of sites can be structured to apply electrical stimulation to a discrete amount of liquid located in individual sites. In the illustrative example of FIG. 15, the semiconductor device 1500 includes a first site comprised of the first cavity 1516, the first pore 1524, the first electrode 1532, the first well electrode 1556, and the well 1562; a second site comprised of the second cavity 1518, the second pore 1526, the second electrode 1534; the second well electrode 1558, and the second well 1564; and a third site comprised of the third cavity 1520, the third pore 1528, the third electrode 1536, the third well electrode 1560, and the third well 1566. The sites can also be defined by sections of the separator layer 1502 that fluidically and electrically isolate liquid disposed in the sites of the semiconductor device 1500. Although the illustrative example of FIG. 15 shows three sites of the semiconductor device 1500, the semiconductor device 1500 can include a greater number of sites. For example, the semiconductor device 1500 can include at least 100 sites, at least 500 sites, at least 1000 sites, at least 5000 sites, at least 10,000 sites, at least 25,000 sites, at least 50,000 sites, at least 100,000 sites, or more.

In one or more examples, a volume of liquid can be stored in the wells 1562, 1564, 1566. In one or more illustrative examples, volumes of liquid disposed in the individual wells 1562, 1564, 1566 can include from about 1 picoliter (pL) to about 100 nanoliters (nL), from about 5 pL to about 10 nL, from about 10 pL to about 1 nL from about 1 pL to about 100 pL, from about 1 pL to about 50 pL, from about 1 pL to about 10 pL, from about 10 pL to about 500 pL, from about 10 pL to about 100 pL, from about 10 pL to about 50 pL, from about 50 pL to about 500 pL, from about 50 pL to about 250 pL, from about 100 pL to about 1 nL, from about 100 pL to about 500 pL, from about 500 pL to about 5 nL, from about 500 pL to about 2 nL, from about 500 pL to about 1 nL, from about 1 nL to about 100 nL, from about 1 nL to about 50 nL, or from about 1 nL to about 10 nL.

In the illustrative example of FIG. 15, an amount of liquid 1568 can be dispensed into the second well 1564. Although the illustrative example of FIG. 15 shows the amount of liquid 1568 being dispensed into the second well 1564, additional amounts of the liquid can also be dispensed into the first well 1562, the third well 1566, and other sites of the semiconductor device 1500. In at least some examples, the amount of liquid 1568 can comprise an aqueous solution having one or more biological cells, such as the example biological cell 1570. In various examples, the amount of liquid 1568 can form a droplet in the second well 1564. For example, in scenarios where the walls 1544, 1546 are comprised of hydrophobic materials and the coating 1510 disposed around the pore 1526 is comprised of a hydrophilic material, the amount of liquid 1568 can bead into droplets on the hydrophilic portions of the site and move away from the walls 1544, 1546. In this way, the droplet of the amount of liquid 1568 in the second well 1564 can be electrically and fluidically isolated from droplets of liquid disposed in other sites of the semiconductor device 1500. Accordingly, independent electrophysiological measurements can be produced for individual sites of the semiconductor device 1500. In addition to one or more biological cells 1570, the amount of liquid 1568 can also include an aqueous solution comprising one or more therapeutic compounds, one or more additional reagents, one or more combinations thereof, and the like. In at least some examples, the fluid disposed within the cavities 1516, 1518, 1520 can include reagents that are used in relation to patch clamp processes and can include, in at least some examples, one or more therapeutic compounds.

In various examples, obtaining patch clamp measurements can include applying a pressure difference between the fluid disposed in the wells 1562, 1564, 1566 and the fluid disposed in the cavities 1516, 1518, 1520 to move biological cells 1570 toward the respective pores 1524, 1526, 1528. In one or more examples, the biological cells 1570 can form a seal with the pores 1524, 1526, 1528 such that the biological cells 1570 are disposed at least partially within the pores 1524, 1526, 1528 and on the portions of the coated passivation layer(s) 1510 disposed around the pores 1524, 1526, 1528. In at least some examples, a first pressure difference can be applied to move the biological cells 1570 to form seals with the pores 1524, 1526, 1528 and a second pressure difference can be applied to cause rupturing of the membranes of the biological cells 1570. In one or more illustrative examples, the pressure difference can correspond to a negative pressure. In still other examples, a single pressure difference can be applied to move the biological cells 1570 toward the respective pores 1524, 1526, 1528 and to disrupt the membranes of the biological cells 1570.

While the biological cells 1570 are pressed against the coated one or more passivation layers 1508 using the second amount of pressure to disrupt the membranes of the biological cells 1570, a potential difference can be applied between the reference electrode 1540 and the well electrodes 1556, 1558, 1560. In these scenarios, voltage clamp measurements can be collected. In one or more additional examples, while the biological cells 1570 are pressed against the coated one or more passivation layers 1508 using the second amount of pressure to disrupt the membranes of the biological cells 1570, the well electrodes 1556, 1558, 1560 can deliver an amount of current to the biological cells 1570 through the disrupted membranes of the biological cells 1570 to collect current clamp measurements. The measurements collected during the voltage clamp and/or current clamp operations can be used to determine activity of biological cells 1570 with respect to one or more therapeutic compounds and/or to one or more experimental conditions. In various examples, the amount of activity of the biological cells 1570 during patch clamp operations can indicate a responsiveness of the biological cells 1570 to at least one of the one or more therapeutic compounds or the one or more experimental conditions.

In various examples, the patch clamp measurements can take place while the amount of liquid 1568 is at least substantially stationary within the fluid chamber 1514. In one or more additional examples, the amount of liquid 1568 and one or more other liquids can be in motion within the fluid chamber 1514. For examples, one or more wash solutions and/or one or more buffer solutions can be passed through the fluid chamber 1514. In one or more additional examples, the semiconductor device 1500 can be used to obtain ligand-gated measurements. In these scenarios, one or more solutions including one or more ligands can be passed through the fluid chamber 1514.

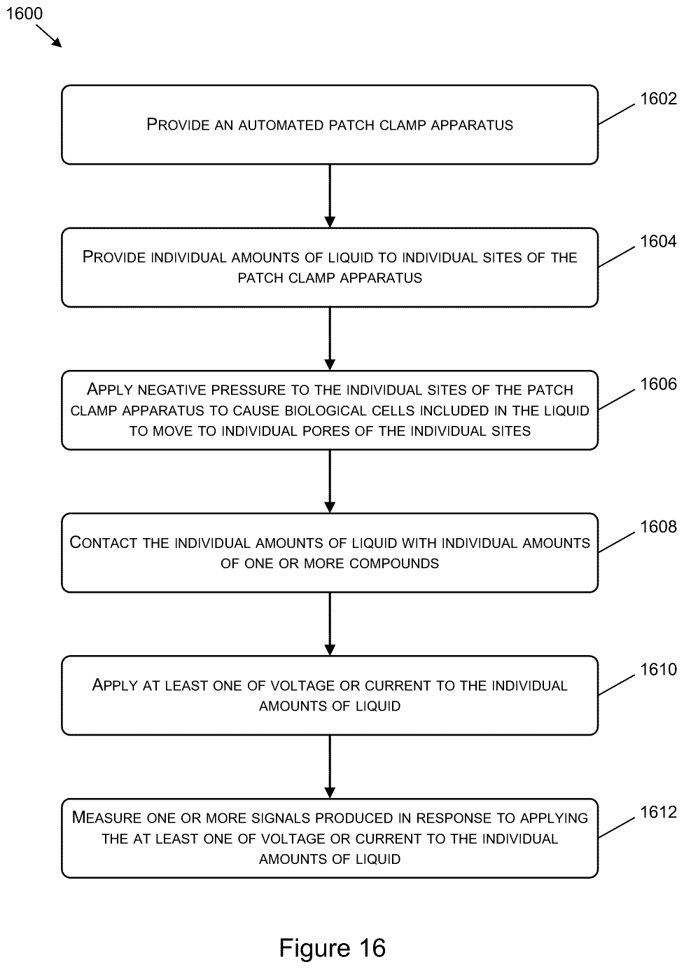

FIG. 16 is a flow diagram of a process 1600 to perform measurements produced in response to electrical stimulation of material disposed in liquids, in accordance with one or more example implementations. At 1602, the process 1600 can include providing an automated patch clamp apparatus. The automated patch clamp apparatus can include a substrate including one or more layers. The one or more layers can include a complementary metal oxide semiconductor (CMOS) substrate. The CMOS substrate can include circuitry to at least one of apply voltage or current to liquid disposed on the CMOS substrate. The CMOS substrate can also include circuitry to measure signals produced in response to the at least one of voltage or current being applied to the liquid. Additionally, the CMOS substrate can include one or more sites. Individual sites of the one or more sites can hold a discrete amount of liquid. The individual sites can include an individual electrode to contact the discrete amount of liquid. The electrode can be electrically coupled to the circuitry of the CMOS substrate. Further, the individual sites can include one or more pores to provide fluid communication between the discrete amount of liquid and one or more additional liquids. In one or more examples, the one or more additional liquids can be disposed in one or more channels of the patch clamp apparatus. In one or more illustrative examples, the automated patch clamp apparatus can correspond to one or more of the semiconductor devices described in relation to FIG. 1-15.

Although, in various examples, the sites of the automated patch clamp apparatus can be physically separated, such as by one or more walls comprised of one or more polymeric regions, in other cases, the sites can be formed using other techniques. To illustrate, biological cells can be grown directly onto a substrate and diced into separate sites. A grid-like mechanical dicer can be used to dice the cells. In one or more illustrative examples, the dicer can be formed using micro-electrical-mechanical-systems (MEMS) techniques. In at least some examples, the dicer can remain after forming the separate biological cells sites to provide a fluidic and electrical barrier between sites.

The process 1600 can also include, at 1604, providing individual amounts of liquid to individual sites of the patch clamp apparatus. The individual amounts of liquid can include one or more biological cells. In one or more examples, the individual amounts of liquid can include an aqueous solution. In various examples, the individual amounts of liquid can include at least one of NaCl, KCl, HEPES buffer, MgCl₂, CaCl₂, glucose, or ligands. In at least some examples, prior to the individual amounts of liquid being provided to the individual sites, cavities and/or channels of the patch clamp apparatus can be perfused with one or more wash solutions and/or one or more buffer solutions. In one or more illustrative examples, the biological cells can be cultured within the patch clamp apparatus by supplying nutrients and growth factors to the patch clamp apparatus.

In addition, at 1606, the process 1600 can include applying a pressure difference to individual sites of the patch clamp apparatus to cause biological cells included in the liquid to move to individual pores of the individual sites. In one or more illustrative examples, the pressure difference can correspond to an amount of negative pressure. The pressure difference applied to move the biological cells to the pores can have a magnitude from about 1 kilopascal (kPa) to about 50 kPa, from about 1 kPa to about 10 kPa, from about 10 kPa to about 20 kPa, from about 20 kPa to about 30 kPa, from about 30 kPa to about 40 kPa, or from about 40 kPa to about 50 kPa. In at least some examples, the pressure difference can cause the membranes of the biological cells to be disrupted. In one or more examples, the biological cells can form a seal with the pores.

In various examples, after applying a pressure difference to draw the biological cells toward the pores, excess fluid can be removed from portions of the patch clamp apparatus. For example, excess fluid can be removed from a top surface of the CMOS substrate. The removal of excess fluid from the top surface of the CMOS substrate can minimize or prevent chemical cross-talk between discrete volumes of liquid located in the individual sites. In one or more illustrative examples, the excess liquid can be removed using one or more aspiration techniques. In one or more additional illustrative examples, the excess liquid can be removed by moving one or more physical structures across the surface of the CMOS substrate. The one or more physical structures can operate similar to that of a squeegee. In one or more further illustrative examples, the one or more physical structures can include a lid having a number of pillars that are disposed over one or more cavities and/or one or more trenches of the automated patch clamp apparatus. The pillars can be pressed over the surfaces of the patch clamp apparatus including within the cavities or trenches to remove excess liquid from these areas. In one or more additional examples, the pillars can also be disposed within trenches or cavities while fluid is flowing through the automated patch clamp apparatus to minimize the amount of liquid filling the trenches or cavities.

In at least some examples, unsealed pores can be identified using at least one of resistance measurements, capacitance measurements, or measurement of additional electrical activity. In one or more examples, sites in which an unsealed pore is present can be short-circuited or otherwise electrically isolated to minimize or prevent electrical interference from the unsealed pore site to amounts of liquid disposed in other sites that do include sealed pores. In one or more additional examples, unsealed pores can be identified and compounds being tested in relation to the biological cells may not be added to the sites where unsealed pores are located. In this way, the sites with unsealed pores can be skipped in relation to the patch clamp measurements. In one or more further examples, plugs can be disposed in the unsealed pores, such as by applying the pressure difference to draw the plugs into the unscaled pores. The plugs can be disposed in fluid that is delivered to the patch clamp apparatus. In still other examples, unscaled pores can be sealed using a curable polymeric material, such as an ultraviolet (UV) electromagnetic radiation curable polymeric material, or by melting the material around the unsealed pores to cause sealing of the unscaled pores.

In various additional illustrative examples, capillary pressure can be used to counteract the negative pressure being applied in the patch clamp apparatus with regard to unsealed pores. Using hydrophilic materials around the pores can minimize or prevent air bubbles from forming in relation to unsealed pores when negative pressure is applied to the automated patch clamp apparatus. In various further illustrative examples, fluid motion in unsealed pores can be opposed using electroosmosis. Electroosmosis can include generating an electric field at the location of the unsealed pore to prevent fluid from moving through the unscaled pore. In one or more scenarios, the electric field can generate a force on the side walls of microfluidic channels that prevents or minimizes flow of fluid out of the site through the pore. In one or more additional implementations, electrodes can be present in the channels to strengthen the electric field and increase the effectiveness of fluid emptying from the site through the unsealed pore and created air bubbles or other disruptions to the automated patch clamp apparatus.

Further, the process 1600 can include, at 1608, contacting the individual amounts of liquid with individual amounts of one or more compounds. At 1610, the process 1600 can include applying at least one of voltage or current to the individual amounts of liquid. In one or more examples, the one or more compounds can include therapeutic compounds. In at least some examples, the liquid including the biological cells can be contacted with liquid including the therapeutic compounds to determine an impact of the therapeutic compounds on the biological cells in response to the electrical stimulation. In various examples, individual sites can have individual circuitry components to apply the electrical stimulation.

Additionally, the process 1600 can include, at 1612, measuring one or more signals produced in response to applying the at least one of voltage or current to the individual amounts of liquid. In at least some examples, the strength of the signals produced in response to the voltage and/or current can indicate a responsiveness of the one or more biological cells to the one or more therapeutic compounds. In one or more illustrative examples, the process 1600 can include following patch clamp protocols that include applying a voltage protocol across different concentrations of the one or more compounds and analyzing an IV curve that corresponds to gate function measured at peak current values. In at least some cases, temporal aspects of the response signal can also be analyzed. Examples of patch clamp protocols that can be applied in relation to biological cells included in the sites of the patch clamp apparatus can include those described in Seibertz, F., Rapedius, M., Fakuade, F. E. et al. A modern automated patch-clamp approach for high throughput electrophysiology recordings in native cardiomyocytes. Commun Biol 5, 969 (2022), which is incorporated by reference herein in its entirety.

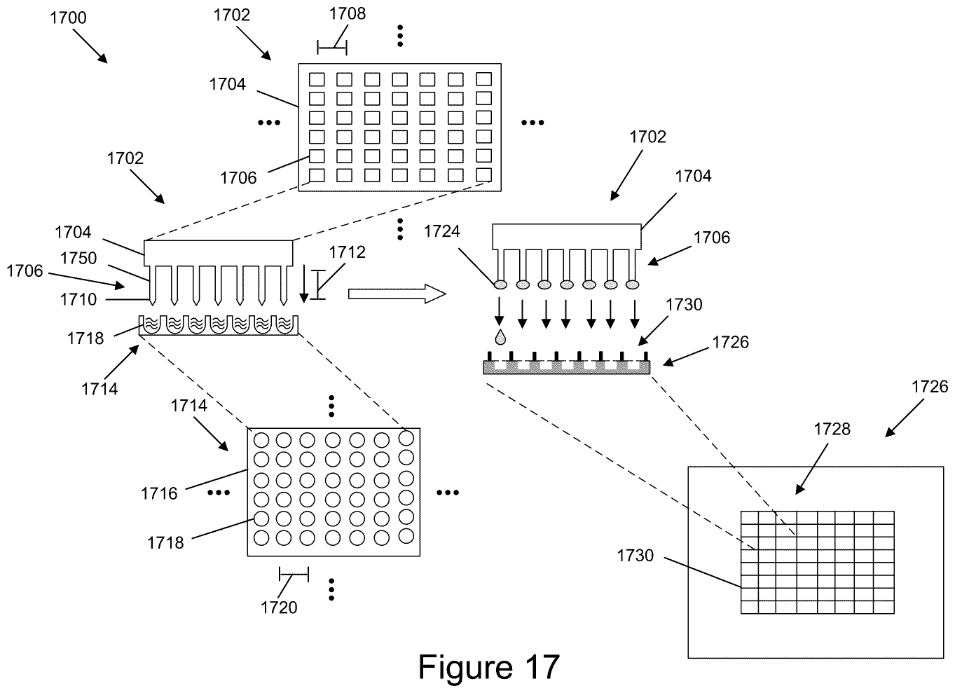

FIG. 17 is a diagram depicting a framework 1700 including a fluid delivery device 1702 that delivers discrete amounts of liquid, in accordance with one or more example implementations. The fluid delivery device 1702 can include a substrate 1704 and a number of fluid delivery structures 1706 disposed on a surface of the substrate 1704. In one or more examples, the number of fluid delivery structures 1706 can be arranged in an array of rows and columns. The fluid delivery structures 1706 can be arranged according to a spacing 1708. The spacing 1708 can correspond to a distance between adjacent fluid delivery structures 1706. In various examples, the spacing 1708 can be measured from a center of a first fluid delivery structure to a center of a second fluid delivery structure. In at least some examples, the spacing 1708 can also be referred to as a pitch. In one or more illustrative examples, the spacing 1708 can be from about 100 μm to about to about 10 millimeters (mm), from about 100 μm to about 5 mm, from about 500 μm to about 2 mm, from about 100 μm to about 500 μm, from about 500 μm to about 1 mm, from about 100 μm to about 300 μm, from about 300 μm to about 500 μm, from about 500 μm to about 700 μm, from about 700 μm to about 900 μm, from about 1 mm to about 3 mm, from about 2 mm to about 4 mm, from about 3 mm to about 5 mm, from about 1 to about 8 mm, from about 4 mm to about 6 mm, from about 1 mm to about 4 mm, or from about 4 mm to about 8 mm.

Individual fluid delivery structures 1706 can include an elongated member 1750 extending from a surface of the substrate 1704 and ending in a tip 1710. In one or more examples, the fluid delivery structures 1706 can be formed from a same material as a material of the substrate 1704. For example, the fluid delivery structures 1706 and the substrate 1704 can be formed using one or more molding processes or one or more extrusion processes. In one or more additional examples, the fluid delivery structures 1706 can be attached to the substrate 1704. To illustrative, the fluid delivery structures 1706 can be attached to the substrate 1704 using one or more adhesives or one or more bonding techniques. In one or more further examples, the fluid delivery structures 1706 can be disposed in holes formed in the substrate 1704. In one or more illustrative examples, the fluid delivery structures 1706 can comprise pins. In one or more illustrative example, the fluid delivery device 1702 can be manufactured using one or more MEMS-related, wafer scale processing technologies. In various examples, the fluid delivery device 1702 can be formed from at least one of one or more polymeric materials, one or more metallic materials, or one or more semiconductor materials.

The fluid delivery structures 1706 can have a length 1712 that is from about 0.1 mm to about 50 mm, from about 1 mm to about 30 mm, from about 5 mm to about 20 mm, from about 0.1 mm to about 1 mm, from about 1 mm to about 2 mm, from about 2 mm to about 5 mm, from about 5 mm to about 10 mm, from about 10 mm to about 15 mm, from about 15 mm to about 20 mm, from about 20 mm to about 25 mm, or from about 25 mm to about 30 mm. The tip 1710 of the fluid delivery structure 1706 can also have a width from about 1 μm to about 10 mm, from about 5 μm to about 5 mm, from about 10 μm to about 1 mm, from about 100 μm to about 500 μm, from about 500 μm to about 1 mm, from about 1 μm to about 100 μm, from about 100 μm to about 300 μm, from about 300 μm to about 500 μm, from about 500 μm to about 700 μm, or from about 700 μm to about 900 μm. The tip 1710 of the fluid delivery structures 1706 can have a specified shape. To illustrate, the tip 1710 can have a triangular shape, a circular shape, or a rectangular shape. In one or more additional examples, at least a first portion of the tips of first fluid delivery structures can have shapes that are different with respect to a second portion of the tips of second fluid delivery structures of the fluid delivery device 1702.

The fluid delivery device 1702 can obtain an amount of liquid from a liquid source 1714 and transfer the amount of liquid to a liquid destination. The liquid source 1714 can includes a substrate 1716 having a number of liquid containers 1718. In one or more illustrative examples, the liquid source 1714 can correspond to a well plate having and the liquid containers 1718 can include individual wells of the well plate. The liquid containers 1718 can be separated by a spacing 1720. In at least some examples, the spacing 1720 can be at least substantially similar or the same with respect to the spacing 1708 of the fluid delivery structures 1706 of the fluid delivery device 1702. In one or more illustrative examples, the spacing 1720 can be from about 100 μm to about to about 10 millimeters (mm), from about 100 μm to about 5 mm, from about 500 μm to about 2 mm, from about 100 μm to about 500 μm, from about 500 μm to about 1 mm, from about 100 μm to about 300 μm, from about 300 μm to about 500 μm, from about 500 μm to about 700 μm, from about 700 μm to about 900 μm, from about 1 mm to about 3 mm, from about 2 mm to about 4 mm, from about 3 mm to about 5 mm, from about 1 to about 8 mm, from about 4 mm to about 6 mm, from about 1 mm to about 4 mm, or from about 4 mm to about 8 mm. In implementations where the liquid source 1714 is a well plate, the liquid source 1714 can include at least 20 wells, at least 50 wells, at least 100 wells, at least 200 wells, at least 500 wells, at least 1000 wells, at least 2000 wells, at least 5000 wells, at least 10,000 wells, or more.

In one or more examples, one or more compounds can be disposed in the liquid containers 1718 of the liquid source 1714. In various examples, the one or more compounds can be therapeutic compounds that can be provided to a patient to treat one or more biological conditions. In at least some examples, the one or more compounds are candidates for treating one or more biological conditions that are to be evaluated using an automated patch clamp system. In one or more illustrative examples, each liquid container 1718 of an individual row or and individual column can include a solution with a same compound. For example, a first row of the liquid containers 1718 can include a solution having a first compound and a second row of the liquid containers 1718 can include a solution having a second compound.

The fluid delivery structures 1706 can be lowered into the liquid containers 1718 of the liquid source 1714 for a period of time sufficient to wet the tips 1710 and then raised out of the liquid containers 1718. The raising and lowering of the fluid delivery structures 1706 with respect to the liquid containers 1718 can be performed using one or more automated or robotic systems. Drops 1724 can be formed on the tips 1710 of the fluid delivery structures 1706 after the fluid delivery structures 1706 are placed in the liquid containers 1718.

In various examples, a destination for the liquid disposed on the fluid delivery structures 1706 can include a semiconductor device 1726. The semiconductor device 1726 can include an array of sites 1728 that includes a number of individual sites 1730. In one or more illustrative examples, the semiconductor device 1726 can correspond to an implementation of the semiconductor devices described in relation to FIGS. 1-15.

In one or more additional illustrative examples, the drops 1724 can be dispensed from tips 1710 of fluid delivery structures 1706 into sites of 1730 of the semiconductor device 1726. In one or more examples, sonication can be used to dislodge the drops 1724 from the fluid delivery structures 1706. In one or more additional examples, a mechanism internal to the substrate 1704 can apply pressure to dislodge the drops 1724 from the fluid delivery structures 1706. After the drops 1724 have been dispensed into the sites 1730, one or more patch clamp measurements can be performed in relation to one or more compounds disposed in the drops 1724. In at least some examples, the spacing 1708 of the fluid delivery structures 1706 can be different from a spacing between the sites 1730. For example, a pitch of the fluid delivery structures 1706 can be different from a pitch of the sites 1730. In these situations, the fluid delivery device 1702 can be moved according to one or more offsets to enable drops 1724 to be dispensed in each of the sites 1730.

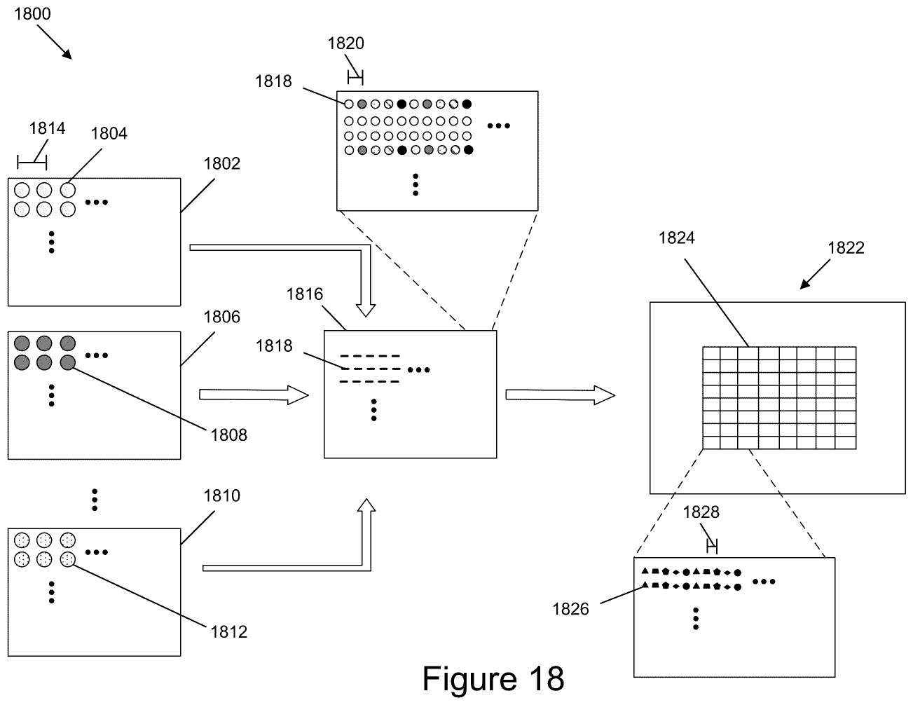

FIG. 18 is a diagram of a process 1800 to transfer liquid from liquid sources disposed at a first spacing to liquid destinations disposed at a second spacing that is smaller than the first spacing, in accordance with one or more example implementations. The process 1800 can include providing a number of liquid sources. For example, the process 1800 can include providing a first liquid source 1802 having a number of first containers 1804, a second liquid source 1806 having a number of second containers 1808, up to an Nth liquid source 1810 having a number of additional containers 1812. In one or more illustrative examples, the liquid sources 1802, 1806, 1810 can comprise well plates and the containers 1804, 1808, 1812 can include individual wells. In various examples, liquids containing different compounds can be stored in the liquid sources 1802, 1806, 1810. To illustrate, the first containers 1804 of the first liquid source 1802 can hold a first liquid including one or more first compounds, the second containers 1808 of the second liquid source 1806 can hold a second liquid including one or more second compounds, and the additional containers 1812 of the Nth liquid source 1810 can hold an additional liquid including one or more additional compounds.

In various examples, the containers 1804, 1808, 1812 can be disposed on the liquid sources 1802, 1806, 1810 according to a pattern, such as a number of rows and columns, having a spacing 1814 between containers. The spacing 1814 can correspond to a pitch of the liquid sources 1802, 1806, 1810. The spacing 1814 can be from about 2 mm to about 10 mm, from about 3 mm to about 8 mm, from about 4 mm to about 6 mm, from about 2 mm to about 4 mm, from about 5 mm to about 7 mm, or from about 6 mm to about 8 mm.

Although the illustrative example of FIG. 18 include three liquid sources, in one or more additional implementations, the process 1800 can be performed with a greater number of liquid sources. For example, the process 1800 can be performed with at least 4 liquid sources, at least 8 liquid sources, at least 12 liquid sources, at least 15 liquid sources, at least 20 liquid sources, at least 25 liquid sources, at least 30 liquid sources, at least 35 liquid sources, or at least 40 liquid sources. In one or more examples, the number of liquid sources used in the process 1800 can correspond to a level of demagnification that is intended to take place by implementing the process 1800 to transfer liquid from liquid sources having containers arranged with a first spacing to a liquid destination having containers arranged according to an additional spacing that is less than the first spacing.

The process 1800 can also include transferring liquid stored by the liquid sources 1802, 1806, up to 1810 to at least one intermediate liquid source 1816. The intermediate liquid source 1816 can include a number of containers 1818 that are arranged according to a pattern, such as a number of rows and columns, having a spacing 1820. The spacing 1820 can be less than the spacing 1814. In one or more illustrative examples, the spacing 1820 can be from about 400 μm to about 1500 μm, from about 500 μm to about 1200 μm, from about 600 μm to about 1000 μm, from about 400 μm to about 800 μm, from about 800 μm to about 1200 μm, from about 400 μm to about 600 μm, from about 600 μm to about 800 μm, from about 800 μm to about 1000 μm, or from about 1000 μm to about 1200 μm. Additionally, the intermediate liquid source 1816 can include a greater number of containers than the liquid sources 1802, 1806, up to 1810. For examples, the intermediate liquid source 1816 can include at least 500 containers, at least 1000 containers, at least 2500 containers, at least 5000 containers, at least 7500 containers, at least 10,000 containers, at least 12,500 containers, or at least 15,000 containers.

In one or more examples, the transfer of liquid from the liquid sources 1802, 1806, up to 1812 can be performed using one or more automated or robotic liquid transfer systems or one or more pin-based liquid transfer systems. For example, one or more automated or robotic liquid transfer systems can be used to transfer liquid from the liquid sources 1802, 1806, up to 1810 to the intermediate liquid source 1816. In at least some examples, liquid can be transferred from one liquid source 1802, 1806, up to 1810 at a time to the intermediate liquid source 1816. In one or more additional examples, liquid can be transferred from multiple sources 1802, 1806, up to 1810 at a time to the intermediate liquid source 1816.

In one or more illustrative examples, because the spacing 1820 is less than the spacing 1814, liquid can be transferred from a single one of the containers 1804 of the first liquid source 1802 to multiple ones of the containers 1818 of the intermediate liquid source 1816. In one or more further illustrative examples, the automated liquid transfer system used to transfer liquid from the liquid sources 1802, 1806, up to 1810 to the intermediate liquid source 1816 can have a pitch that is greater than the spacing 1820. In these situations, the automated liquid transfer system can make distributions of liquid into containers 1818 of the intermediate liquid source 1816 according to one or more offsets. For example, the automated liquid transfer system can distribute liquid from the first liquid source 1802 into a first number of containers of the intermediate liquid source 1816 according to a pitch of the automated liquid transfer system. The liquid transfer system can then move vertically or horizontally according to an offset related to the difference between the spacing 1820 and the pitch of the automated liquid transfer system to deposit an amount of liquid in a second number of containers of the intermediate liquid source 1816 that are individually adjacent to the first number of containers of the intermediate liquid source 1816. In this way, regions of containers of the intermediate liquid source 1816 storing the same liquid can be produced during the transfer of liquid from the liquid sources 1802, 1806, 1810 to the intermediate liquid source 1816.

Although the illustrative example of FIG. 18 includes a single intermediate liquid source 1816, in one or more additional implementations, the process 1800 can be performed using a greater number of intermediate liquid sources, such as at least 2 intermediate liquid sources, at least 5 intermediate liquid sources, at least 10 intermediate liquid sources, at least 20 intermediate liquid sources, at least 50 intermediate liquid sources, or at least 100 intermediate liquid sources.

The process 1800 can also include transferring liquid from one or more intermediate liquid sources 1816 to a liquid destination. The liquid destination can include a semiconductor device 1822 having an array of sites 1824. The semiconductor device 1822 can be one or more of the example semiconductor devices described in relation to FIGS. 1-15.

The semiconductor device 1822 can include a number of individual sites 1826 that are arranged according to a spacing 1828. The spacing 1828 can be less than the spacing 1820 of the intermediate liquid source 1816. In one or more illustrative examples, the spacing 1828 can be from about 10 μm to about 500 μm, from about 50 μm to about 400 μm, from about 100 μm to about 300 μm, from about 50 μm to about 250 μm, from about 100 μm to about 300 μm, from about 200 μm to about 400 μm, from about 100 μm to about 200 μm, or from about 200 μm to about 300 μm.

In one or more examples, liquid can be transferred to from one or more intermediate liquid sources 1816 to a liquid destination using an automated or robotic liquid transfer system. In one or more illustrative examples, because the spacing 1828 is less than the spacing 1820, liquid can be transferred from a single one of the containers 1818 of the intermediate liquid source 1816 to multiple ones of the sites 1826 of the semiconductor device 1822. In one or more further illustrative examples, the automated liquid transfer system used to transfer liquid from the intermediate liquid source 1816 to the semiconductor device 1822 can have a pitch that is greater than the spacing 1828. In these situations, the automated liquid transfer system can make distributions of liquid into the sites 1826 of the semiconductor device 1822 according to one or more offsets. For example, the automated liquid transfer system can distribute liquid from the intermediate liquid source 1816 into a first number of sites of the semiconductor device 1822 according to a pitch of the automated liquid transfer system. The liquid transfer system can then move vertically or horizontally according to an offset related to the difference between the spacing 1828 and the pitch of the automated liquid transfer system to deposit an amount of liquid in a second number of sites of the semiconductor device 1822. In at least some examples, the automated liquid handling system can produce regions of sites of the semiconductor device 1822 storing the same liquid during the transfer of liquid from the intermediate liquid source 1816 to the semiconductor device 1822.

In further illustrative examples, a system used to perform the transfer of liquid from liquid sources 1802, 1806, up to 1810 to the semiconductor device 1822 can track the locations of different liquids in the containers of the liquid handling substrates. For example, a system can identify first sites of the semiconductor device 1822 that are associated with a first liquid including one or more first compounds stored by the first liquid source 1802, second sites of the semiconductor device 1822 that are associated with a second liquid including one or more second compounds stored by the second liquid source 1806, and additional sites of the semiconductor device 1822 that are associated with an additional liquid including one or more additional compounds stored by the Nth liquid source 1810. The system can also determine the containers of one or more intermediate liquid sources 1816 in which the first liquid, the second liquid, and the additional liquid are stored. Thus, the system used to implement the process 1800 can track the location of different liquids in each of the liquid sources 1802, 1806, 1810, 1816 and, based on information indicating the sites 1826 corresponding to each liquid, the system can withdraw and deposit liquids in a manner to ensure that the proper liquid is distributed to the individual sites 1826.

A numbered non-limiting list of aspects of the present subject matter is presented below.

Aspect 1. A semiconductor device comprising: a substrate including one or more layers and one or more sites formed by at least a portion of the one or more layers, the one or more layers including a complementary metal oxide semiconductor (CMOS) layer; wherein the CMOS layer includes: circuitry to (i) apply at least one of voltage or current to a plurality of electrodes coupled to the circuitry and (ii) measure signals produced in response to the at least one of voltage or current being applied to the plurality of electrodes; and wherein individual sites of the one or more sites: are configured to hold a discrete amount of liquid; include one or more electrodes of the plurality of electrodes, the one or more electrodes contacting the discrete amount of liquid stored by the individual site; and include one or more pores to provide fluid communication between the discrete amount of liquid and one or more additional liquids stored by the individual site.

Aspect 2. The semiconductor device of aspect 1, comprising one or more cavities formed by at least a portion of the CMOS layer, individual cavities of the one or more cavities corresponding to an individual site of the one or more sites and wherein the one or more additional liquids are disposed in the individual cavities.

Aspect 3. The semiconductor device of aspect 2, wherein: one or more passivation layers are disposed on the CMOS layer; the one or more pores have a width from about 0.5 micrometers to about 5 micrometers and are formed in the one or more passivation layers; and the one or more passivation layers overlay a portion of the individual cavities of the one or more cavities.

Aspect 4. The semiconductor device of aspect 3, wherein the one or more passivation layers are comprised of at least one of silicon nitride or silicon oxide and have a thickness from about 0.5 micrometers to about 20 micrometers.

Aspect 5. The semiconductor device of aspect 3 or, wherein: a coating is disposed over (i) the one or more passivation layers and (ii) one or more surfaces of the CMOS layer that form the one or more cavities; and the coating has a thickness from about 10 nanometers to about 1000 nanometers.

Aspect 6. The semiconductor device of any one of aspects 3-5, wherein: a channel is formed between the one or more passivation layers and a lid disposed over the CMOS layer; and a reference electrode is located on the lid and within the channel.

Aspect 7. The semiconductor device of any one of aspect 2-6, comprising: a channel in fluid communication with the one or more cavities and disposed below the CMOS layer, wherein at least one of a reference electrode or a current carrying electrode are disposed in the channel.

Aspect 8. The semiconductor device of any one of aspects 2-7, comprising: a plurality of polymer regions disposed on the CMOS layer, wherein (i) the plurality of polymer regions define wells that store the discrete amounts of liquid and (ii) individual wells are in fluid communication with individual cavities of the one or more cavities.

Aspect 9. The semiconductor device of aspect 8, wherein the plurality of polymer regions are comprised of one or more hydrophobic polymeric materials.

Aspect 10. The semiconductor device of any one of aspects 2-9, comprising: a silicon-on-insulator substrate disposed on the CMOS layer; wherein: one or more additional cavities are formed within the silicon-on-insulator substrate; individual additional cavities of the one or more additional cavities correspond to individual cavities of the one or more cavities; and an individual cavity formed in the CMOS layer and an individual additional cavity formed in the silicon-on-insulator substrate form a channel of an individual site of the one or more sites.

Aspect 11. The semiconductor device of aspect 10, wherein a first portion of an individual electrode corresponding to an individual site of the one or more sites is disposed on the silicon-on-insulator substrate and a second portion of the individual electrode is disposed in a via within the silicon-on-insulator substrate.

Aspect 12. The semiconductor device of aspect 10 or 11, comprising: one or more further cavities formed adjacent to channels of the individual sites, and wherein a measurement electrode is formed in the one or more further cavities.

Aspect 13. The semiconductor device of any one of aspect 2-12, comprising: an additional substrate disposed on the CMOS layer and over the one or more cavities, the additional substrate comprising one or more glass materials.

Aspect 14. The semiconductor device of aspect 13, wherein: a number of through-holes are formed in the additional substrate with individual through-holes of the number of through-holes corresponding to an individual site of the one or more sites; individual through holes being formed adjacent to the one or more cavities and being in fluid communication with the one or more cavities by an individual pore of the one or more pores; individual measurement electrodes are disposed on the CMOS layer and disposed in relation to individual through-holes; and at least one of a reference electrode or a current carrying electrode is disposed in individual cavities of the one or more cavities.

Aspect 15. A process to manufacture semiconductor device, the process comprising: providing a substrate having one or more layers, the one or more layers including a complementary metal oxide semiconductor (CMOS) layer having a first surface and a second surface disposed at least substantially parallel with respect to the first surface; forming circuitry on or within the CMOS layer, the circuitry to (i) apply at least one of voltage or current to a plurality of electrodes and (ii) measure signals produced in response to the at least one of voltage or current being applied to the plurality of electrodes; forming one or more passivation layers on at least one of the first surface or the second surface of the CMOS layer forming a number of sites from the one or more layers of the substrate, individual sites of the number of sites being configured to hold a discrete amount of liquid and including one or more electrodes of the plurality of electrodes; and producing a number of pores in the one or more passivation layers, individual sites of the number of sites including one or more pores of the number of pores.

Aspect 16. The process of claim 15, comprising: forming a number of cavities within the substrate; and wherein the number of pores within the one or more passivation layers are formed by: forming a pattern of one or more resist materials on the first surface of the CMOS layer, the pattern corresponding to locations of the number of pores within the one or more passivation layers; performing a first etch operation to form partial openings in the one or more passivation layers according to the pattern such that a portion of the one or more passivation layers remain within the partial openings; and performing a second etch operation to form full openings within the one or more passivation layers; wherein individual pores of the number of pores correspond to individual cavities of the number of cavities.

Aspect 17. The process of aspect 15 or 16, wherein the plurality of electrodes are formed by: forming a pattern of one or more resist materials on the one or more passivation layers, the pattern corresponding to locations of the plurality of electrodes on the first surface of the CMOS layer; performing an etch process to form a number of openings in the one or more passivation layers according to the pattern; and depositing an amount of metallic material in the number of openings.

Aspect 18. The process of aspect 16 or 17, comprising: depositing one or more polymeric materials on the one or more passivation layers to form a plurality of wells, individual wells of the plurality of wells being in fluid communication with individual cavities of the number of cavities by an individual pore of a number of pores formed in the one or more passivation layers.

Aspect 19. A process comprising: providing semiconductor device that includes a substrate having a complementary metal oxide semiconductor (CMOS) layer that forms a number of sites, individual sites of the number of sites being configured to perform patch clamp operations; providing individual amounts of liquid to the individual sites; applying a pressure difference to cause one or more biological cells disposed in the individual amounts of liquid corresponding to the individual sites to move to a location of a pore included in the individual sites and to be in contact with an electrode of the individual sites; contacting the individual amounts of liquid with individual amounts of one or more compounds; applying at least one of a voltage or a current to the individual amounts of the liquid using the electrode of the individual sites; and measuring one or more signals produced in response to applying the at least one of the voltage or the current to the individual amounts of the liquid.

Aspect 20. The process of aspect 19, wherein a first individual amount of liquid located in a first site of the number of sites is electrically isolated from a second individual amount of liquid located in a second site of the number of sites.

Each of the non-limiting aspects or examples described herein may stand on its own or may be combined in various permutations or combinations with one or more of the other examples.

The above detailed description includes references to the accompanying drawings, which form a part of the detailed description. The drawings show, by way of illustration, specific embodiments in which the invention may be practiced. These implementations are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other implementations can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed implementation. Thus, the following claims are hereby incorporated into the Detailed Description as examples or implementations, with each claim standing on its own as a separate implementation, and it is contemplated that such implementations can be combined with each other in various combinations or permutations. The scope of the invention should be determined with reference to the appended claims, along with the full scope of equivalents to which such claims are entitled.

What is claimed is:

1. A semiconductor device comprising:
a substrate including one or more layers and one or more sites formed by at least a portion of the one or more layers, the one or more layers including a complementary metal oxide semiconductor (CMOS) layer;
wherein the CMOS layer includes:
circuitry to (i) apply at least one of voltage or current to a plurality of electrodes coupled to the circuitry and (ii) measure signals produced in response to the at least one of voltage or current being applied to the plurality of electrodes; and
wherein individual sites of the one or more sites:
are configured to hold a discrete amount of liquid;
include one or more electrodes of the plurality of electrodes, the one or more electrodes contacting the discrete amount of liquid stored by the individual site; and
include one or more pores to provide fluid communication between the discrete amount of liquid and one or more additional liquids stored by the individual site.

2. The semiconductor device of claim 1, comprising one or more cavities formed by at least a portion of the CMOS layer, individual cavities of the one or more cavities corresponding to an individual site of the one or more sites and wherein the one or more additional liquids are disposed in the individual cavities.

3. The semiconductor device of claim 2, wherein:
one or more passivation layers are disposed on the CMOS layer;
the one or more pores have a width from about 0.5 micrometers to about 5 micrometers and are formed in the one or more passivation layers; and
the one or more passivation layers overlay a portion of the individual cavities of the one or more cavities.

4. The semiconductor device of claim 3, wherein the one or more passivation layers are comprised of at least one of silicon nitride or silicon oxide and have a thickness from about 0.5 micrometers to about 20 micrometers.

5. The semiconductor device of claim 3, wherein:
a coating is disposed over (i) the one or more passivation layers and (ii) one or more surfaces of the CMOS layer that form the one or more cavities; and
the coating has a thickness from about 10 nanometers to about 1000 nanometers.

6. The semiconductor device of claim 3, wherein:
a channel is formed between the one or more passivation layers and a lid disposed over the CMOS layer; and
a reference electrode is located on the lid and within the channel.

7. The semiconductor device of claim 2, comprising:
a channel in fluid communication with the one or more cavities and disposed below the CMOS layer, wherein at least one of a reference electrode or a current carrying electrode are disposed in the channel.

8. The semiconductor device of claim 2, comprising:
a plurality of polymer regions disposed on the CMOS layer, wherein (i) the plurality of polymer regions define wells that store the discrete amounts of liquid and (ii) individual wells are in fluid communication with individual cavities of the one or more cavities.

9. The semiconductor device of claim 8, wherein the plurality of polymer regions are comprised of one or more hydrophobic polymeric materials.

10. The semiconductor device of claim 2, comprising:
a silicon-on-insulator substrate disposed on the CMOS layer;
wherein:
one or more additional cavities are formed within the silicon-on-insulator substrate;
individual additional cavities of the one or more additional cavities correspond to individual cavities of the one or more cavities; and
an individual cavity formed in the CMOS layer and an individual additional cavity formed in the silicon-on-insulator substrate form a channel of an individual site of the one or more sites.

11. The semiconductor device of claim 10, wherein a first portion of an individual electrode corresponding to an individual site of the one or more sites is disposed on the silicon-on-insulator substrate and a second portion of the individual electrode is disposed in a via within the silicon-on-insulator substrate.

12. The semiconductor device of claim 10, comprising:
one or more further cavities formed adjacent to channels of the individual sites, and wherein a measurement electrode is formed in the one or more further cavities.

13. The semiconductor device of claim 2, comprising:
an additional substrate disposed on the CMOS layer and over the one or more cavities, the additional substrate comprising one or more glass materials.

14. The semiconductor device of claim 13, wherein:
a number of through-holes are formed in the additional substrate with individual through-holes of the number of through-holes corresponding to an individual site of the one or more sites;
individual through holes being formed adjacent to the one or more cavities and being in fluid communication with the one or more cavities by an individual pore of the one or more pores;
individual measurement electrodes are disposed on the CMOS layer and disposed in relation to individual through-holes; and
at least one of a reference electrode or a current carrying electrode is disposed in individual cavities of the one or more cavities.

15. A process to manufacture semiconductor device, the process comprising:
providing a substrate having one or more layers, the one or more layers including a complementary metal oxide semiconductor (CMOS) layer having a first surface and a second surface disposed at least substantially parallel with respect to the first surface;
forming circuitry on or within the CMOS layer, the circuitry to (i) apply at least one of voltage or current to a plurality of electrodes and (ii) measure signals produced in response to the at least one of voltage or current being applied to the plurality of electrodes;
forming one or more passivation layers on at least one of the first surface or the second surface of the CMOS layer
forming a number of sites from the one or more layers of the substrate, individual sites of the number of sites being configured to hold a discrete amount of liquid and including one or more electrodes of the plurality of electrodes; and
producing a number of pores in the one or more passivation layers, individual sites of the number of sites including one or more pores of the number of pores.

16. The process of claim 15, comprising:
forming a number of cavities within the substrate; and
wherein the number of pores within the one or more passivation layers are formed by:
forming a pattern of one or more resist materials on the first surface of the CMOS layer, the pattern corresponding to locations of the number of pores within the one or more passivation layers;
performing a first etch operation to form partial openings in the one or more passivation layers according to the pattern such that a portion of the one or more passivation layers remain within the partial openings; and
performing a second etch operation to form full openings within the one or more passivation layers;
wherein individual pores of the number of pores correspond to individual cavities of the number of cavities.

17. The process of claim 15, wherein the plurality of electrodes are formed by:
forming a pattern of one or more resist materials on the one or more passivation layers, the pattern corresponding to locations of the plurality of electrodes on the first surface of the CMOS layer;
performing an etch process to form a number of openings in the one or more passivation layers according to the pattern; and
depositing an amount of metallic material in the number of openings.

18. The process of claim 16, comprising:
depositing one or more polymeric materials on the one or more passivation layers to form a plurality of wells, individual wells of the plurality of wells being in fluid communication with individual cavities of the number of cavities by an individual pore of a number of pores formed in the one or more passivation layers.

19. A process comprising:
providing semiconductor device that includes a substrate having a complementary metal oxide semiconductor (CMOS) layer that forms a number of sites, individual sites of the number of sites being configured to perform patch clamp operations;
providing individual amounts of liquid to the individual sites;
applying a pressure difference to cause one or more biological cells disposed in the individual amounts of liquid corresponding to the individual sites to move to a location of a pore included in the individual sites and to be in contact with an electrode of the individual sites;
contacting the individual amounts of liquid with individual amounts of one or more compounds;
applying at least one of a voltage or a current to the individual amounts of the liquid using the electrode of the individual sites; and
measuring one or more signals produced in response to applying the at least one of the voltage or the current to the individual amounts of the liquid.

20. The process of claim 19, wherein a first individual amount of liquid located in a first site of the number of sites is electrically isolated from a second individual amount of liquid located in a second site of the number of sites.
